(19) United States
(12) Patent Application Publication
(10) Pub. No.: US 2025/0179518 A1
(43) Pub. Date: Jun. 5, 2025

(54) RECOMBINANT ADENO-ASSOCIATED VIRUSES FOR CNS TROPIC DELIVERY

(71) Applicant: REGENXBIO INC., Rockville, MD (US)

(72) Inventors: Olivier Danos, Princeton, NJ (US); Samantha Yost, Rockville, MD (US); Andrew Mercer, Poolesville, MD (US); Ye Liu, Clarksville, MD (US); Joseph Bruder, Gaithersburg, MD (US); Subha Karumuthil-Melethil, Germantown, MD (US); Elad Firnberg, Baltimore, MD (US); Randolph Qian, Gaithersburg, MD (US); April R. Tepe, Laurel, MD (US); Jennifer M. Egley, Gaithersburg, MD (US); Jared B. Smith, North Potomac, MD (US); Bradley Hollidge, Washington, DC (US)

(21) Appl. No.: 18/697,987

(22) PCT Filed: Oct. 7, 2022
(86) PCT No.: PCT/US2022/077821

§ 371 Date: Apr. 2, 2024

Related U.S. Application Data
(60) Provisional application No. 63/253,519, filed on Oct. 7, 2021.
(60) Provisional application No. 63/331,184, filed on Apr. 14, 2022.
(60) Provisional application No. 63/342,354, filed on May 16, 2022.

(51) Int. Cl. C12N 15/86; A61K 48/00; C07K 14/005

(52) U.S. Cl. CPC C12N 15/86; A61K 48/0058; C07K 14/005; C12N 2750/14122; C12N 2750/14143

(57) ABSTRACT

The present invention relates to recombinant adeno-associated viruses (rAAVs) having capsid proteins engineered to include amino acid sequences and/or amino acid substitutions that confer and/or enhance desired properties, particularly increased transduction in CNS upon intraparenchymal administration relative to a rAAV having a reference capsid.

The contents of the electronic sequence listing submitted herewith as file 38013_0023P1FINAL.xml; Size: 135,000 bytes; and Date of Creation: Oct. 7, 2022, is herein incorporated by reference in its entirety.

### 1. FIELD OF THE INVENTION

The present invention relates to recombinant adeno-associated viruses (rAAVs) having capsid proteins with one or more amino acid substitutions and/or peptide insertions that confer and/or enhance desired properties, including tissue tropisms. In particular, the invention provides engineered capsid proteins comprising one or more amino acid substitutions or peptide insertions that enhance the tropism of an AAV serotype for one or more tissue types as well as capsids that are not engineered but are found to confer muscle or CNS tropisms on rAAVs. Particularly, the one or more amino acid substitutions and/or insertions in the AAV capsid improve transduction, genome integration and/or transgene expression in heart and/or muscle tissue or the central nervous system while reducing tropism for the liver and/or the dorsal root ganglion and/or peripheral nerve cells. Provided are engineered capsids which when administered to the striatum, for example by intraparenchymal administration, exhibit a tropism for dopaminergic neurons. rAAVs having the capsid proteins disclosed herein are useful for delivering a transgene encoding a therapeutic protein or therapeutic nucleic acid for treatment of CNS or muscle disease.

### 2. BACKGROUND

Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and over twenty gene therapy products are currently in clinical development. Recombinant AAVs, such as AAV9, have demonstrated desirable neurotropic properties and clinical trials using recombinant AAV9 for treatment of CNS disease are underway. Delivery to muscle and/or heart tissue is also desirable. Reduction of transduction of liver and/or dorsal root ganglion cells may also be desirable to reduce toxicity. However, attempts to enhance the neurotropic or muscle/heart tropic properties of rAAVs in human subjects have met with limited success.

There remains a need for rAAV vectors with enhanced neurotropic properties for use, e.g., in treating disorders associated with the central nervous system are desirable, with minimal transduction in liver and/or dorsal root ganglion cells and/or peripheral nerve cells to minimize adverse effects. There also is a need for rAAV vectors with enhanced tissue-specific targeting and/or enhanced tissue-specific transduction to deliver therapies, such that decreasing dose by several fold (or increasing transgene expression several fold at an equivalent dose) would be highly beneficial.

Disclosed herein are AAV particles (capsids packaging a transgene) administered intraparenchymally to the striatum resulting in delivery of such capsids to other areas of the brain via retrograde and/or anterograde transport.

### 3. SUMMARY OF THE INVENTION

Provided are recombinant adeno-associated viruses (rAAVs) having capsid proteins engineered to have one or more amino acid substitutions and/or peptide insertion that enhance tissue targeting, transduction and/or integration of the rAAV genome in CNS and/or muscle tissue relative to a reference capsid, for example, the parent capsid or an AAV8 or AAV9 capsid, while having reduced biodistribution in certain tissues, such as liver and dorsal root ganglion cells, relative to the distribution in CNS and/or muscle and/or relative to the parent capsid or a reference capsid, such as AAV8 or AAV9 capsid, to reduce toxicity. Biodistribution studies in mice and non-human primates permit assessment of relative transduction and transgene transcription and expression in tissue types of capsids, including engineered capsids (see, Examples 9-11 infra). Accordingly, provided herein are rAAVs with enhanced or increased biodistribution, including transduction, genome integration, transgene transcription and expression, in CNS tissues (including frontal cortex, hippocampus, cerebellum, midbrain) relative to a reference capsid (for example the unengineered, parental capsid that has been modified or AAV8 or AAV9), with reduced distribution, including transduction, genome integration, transgene transcription and expression in the liver and/or dorsal root ganglion cells (cervical, thoracic, and/or lumbar) compared to the biodistribution in CNS tissue and/or relative to an AAV with a reference capsid, such as the parental capsid or AAV8 or AAV9. Also provided herein are rAAVs with enhanced or increased biodistribution, including transduction, genome integration, transgene transcription and expression, in specific CNS tissues, including dopaminergic neurons and tissues including cortex, globus pallidus, amygdala, intralaminar thalamus, and substantia nigra, including when administered to the striatum, relative to a reference capsid (for example the unengineered, parental capsid that has been modified or AAV8 or AAV9), in embodiments, with reduced distribution, including transduction, genome integration, transgene transcription and expression in the liver and/or dorsal root ganglion cells (cervical, thoracic, and/or lumbar) compared to the biodistribution in CNS tissue and/or relative to an AAV with a reference capsid, such as the parental capsid or AAV8 or AAV9. Such rAAVs may be useful to deliver therapeutic proteins or therapeutic nucleic acids for the treatment of CNS disease.

In embodiments, provided are methods of delivering rAAV vectors to the striatum (or caudate or putamen) of a subject by intraparenchymal administration of an rAAV vector having a transgene encoding a therapeutic protein or therapeutic nucleic acid. In embodiments, the rAAV incorporates a modified capsid protein, including an AAV8 or AAV9 capsid protein, that has enhanced transduction and/or transgene transcription or expression of the putamen or striatum, hippocampus, frontal cortex and/or midbrain compared to a rAAV incorporating an unmodified wild type capsid, such as an AAV8 or AAV9 capsid, for example, as measured by relative transgene RNA abundance (or transgene expression or DNA transgene copies). In embodiments, transduction is enhanced in the putamen, caudate ipsi, amydgala, globus pallidus (external) (GPe), or substantia nigra tissue relative to an AAV9 or AAV8 parental capsid (see, e.g., Example 9). In embodiments, transduction (including transgene transcription or expression) is enhanced 2 fold, 5 fold, 10 fold, 50 fold, or 100 fold relative to transduction of an AAV9 or AAV8 parental capsid. In embodiments, the capsid is AAV9.S454.TFR3 (SEQ ID NO: 42) or other engineered capsids disclosed herein. The vectors may be useful in the treatment of a neurological disorder, for example a disorder involving dopaminergic neurons, including movement disorders, such as Parkinson's Disease, levodopa-induced dyskinesia (LID) in Parkinson's Disease (PD-LID), and tardive dyskinesia, or alternatively, schizophrenia.

In addition, provided herein are rAAVs with enhanced or increased biodistribution, including transduction, genome integration, transgene transcription and expression, in skeletal muscle and/or cardiac muscle tissues relative to a reference capsid (for example the unengineered, parental capsid or AAV8 or AAV9), with reduced distribution, including transduction, genome integration, transgene transcription and expression in the liver and/or dorsal root ganglion cells (cervical, thoracic, and/or lumbar) compared to the biodistribution in muscle tissue and/or relative to an AAV with a reference capsid, such as the parental capsid or AAV8 or AAV9. Such rAAVs may be useful to deliver therapeutic proteins or therapeutic nucleic acids for the treatment of muscle disease.

In particular, provided are AAV9 capsid proteins or AAV8 capsid proteins (SEQ ID NO: 74 or 73, respectively, and as numbered in FIG. 7) or having one or more amino acid substitutions (including, 2, 3 or 4 amino acid substitutions) that preferentially (in particular, at a greater level than rAAVs with the wild type AAV8 or AAV9 capsid) transduce (target) cells of the CNS, and, in certain embodiments, do not target or transduce, or have reduced transduction compared to rAAVs with wild type AAV8 or AAV9 capsids, the liver and/or the dorsal root ganglion and/or peripheral nerve cells. In other embodiments, provided are AAV9 capsid proteins or AAV8 capsid proteins (SEQ ID NO:74 or 73, respectively, and as numbered in FIG. 7) having one or more amino acid substitutions (including, 2, 3 or 4 amino acid substitutions) that preferentially (in particular, at a greater level than rAAVs with the wild type AAV9 capsid) transduce (target) cells of the heart and/or skeletal muscle, and, in certain embodiments, do not target or transduce, or have reduced transduction compared to rAAVs with wild type AAV8 or AAV9 capsids, the liver and/or the dorsal root ganglion and/or peripheral nerve cells. Such amino acid modifications include S263F/S269T/A273T of AAV9, and corresponding substitutions in other AAV type capsids (for example according to the alignment in FIG. 7), or W530R, Q474A, N272A, or G266A of AAV9, and corresponding substitutions in other AAV type capsids or A269S of AAV8 and corresponding substitutions in other AAV capsids (for example, according to the alignment in FIG. 7). Also provided are capsids, particularly AAV9 capsids having a peptide TLAAPFK (SEQ ID NO:1) inserted between Q588 and A589 (herein PHP.hDYN) or alternatively between S268 and S269 or between S454 and G455) or inserted in another AAV capsid at a corresponding position (see, e.g., FIG. 7). Or, alternatively, the capsid is an AAV9 PHP.eB capsid (which has the modifications A587D and Q588G and insertion of the peptide TLAVPFK (SEQ ID NO:20) between G588 and A589) and the peptide TILSRSTQTG (SEQ ID NO:15) between position 138 and 139, or the corresponding. Additional capsids have a Kidney 1 peptide LPVAS (SEQ ID NO: 6) inserted into the capsid, for example between S454 and G455 of AAV9, or alternatively between S268 and S269 or between Q588 and A589, or the corresponding position of a different capsid. Also provided are capsids having a TFR3 peptide RTIGPSV (SEQ ID NO:19) inserted into the capsid, for example between S454 and G455 of AAV9, or alternatively between S268 and S269 or between Q588 and A589, or the corresponding position of a different capsid. In some embodiments, the capsids can comprise R697W substitution of AAV rh64R1. The capsids having these amino acid substitutions and insertions may further have substitutions of the NNN (asparagines) at 496 to 498 with AAA (alanines) of the AAV9 capsid or at positions 498 to 500 of the AAV8 capsid, or corresponding substitutions in other AAV type capsids. Engineered capsids include AAV8.BB.LD (A269S,498-NNN/AAA-500 substitutions in the amino acid sequence of AAV8, SEQ ID NO 73), AAV9.BB.LD (S263G/S269T/A273T, 496-NNN/AAA-498 substitutions in the amino acid sequence of AAV9, SEQ ID NO:74), AAV9.496-NNN/AAA-498 (SEQ ID NO:31), AAV9.496-NNN/AAA-498.W503R (SEQ ID NO:32), AAV9.W503R (SEQ ID NO:33), or AAV9.Q474A (SEQ ID NO:34). In other examples, the capsid can be AAV9.N272A.496-NNN-498 (SEQ ID NO:49) or AAV9.G266A.496-NNN-498 (SEQ ID NO:50). In other embodiments, the capsid is AAV9.S454.TFR3 (SEQ ID NO:42). In other embodiments, the capsid is not an engineered capsid, but is an AAVrh. 10 capsid (SEQ ID NO:76), an AAVrh.46 capsid (SEQ ID NO:97), an AAVrh.64.R1 capsid (SEQ ID NO:48) or an AAVrh.73 capsid (SEQ ID NO:79). In certain embodiments, transduction is measured by detection of transgene, such as GFP fluorescence.

The capsid protein to be engineered may be an AAV9 capsid protein but may also be any AAV capsid protein, such as AAV serotype 1 (SEQ ID NO:63); AAV serotype 2 (SEQ ID NO: 64); AAV serotype 3 (SEQ ID NO:65), AAV serotype 3-3 (SEQ ID NO: 66); AAV serotype 3B (SEQ ID NO:67); AAV serotype 4 (SEQ ID NO:68); AAV serotype 4-4 (SEQ ID NO: 69); AAV serotype 5 (SEQ ID NO:70); AAV serotype 6 (SEQ ID NO:71); AAV serotype 7 (SEQ ID NO:72); AAV serotype 8 (SEQ ID NO:73); AAV serotype 9 (SEQ ID NO:74); AAV serotype 9e (SEQ ID NO:75); AAV serotype rh10 (SEQ ID NO:76); AAV serotype rh20 (SEQ ID NO:77); and AAV serotype hu.37 (SEQ ID NO:85), AAV serotype rh39 (SEQ ID NO: 78), and AAV serotype rh74 (SEQ ID NO:80 or SEQ ID NO:81), AAV serotype rh.34 (SEQ ID NO:88), AAV serotype hu.60, AAV serotype rh.21 (SEQ ID NO:93), AAV serotype rh. 15, AAV serotype rh.24, AAV serotype hu.5, AAV serotype hu.10, AAV serotype rh64R1 (SEQ ID NO:48), AAV serotype rh46 (SEQ ID NO:97), and AAV serotype rh73 (SEQ ID NO: 79) (see FIG. 7 for alignment of certain sequences) and Table 17 for sequences. In some embodiments, the capsids of these vectors are not engineered. For example, unmodified AAV serotype rh64R1 (SEQ ID NO:48) AAV serotype rh. 10 ((SEQ ID NO:76) AAV serotype rh46 (SEQ ID NO:97), and AAV serotype rh73 (SEQ ID NO:79) can be used in the disclosed methods and compositions.

In certain embodiments, provided are rAAVs incorporating the engineered capsids described herein, including rAAVs with genomes comprising a transgene of therapeutic interest, including a transgene encoding a therapeutic protein or therapeutic nucleic acid for treatment of a muscle, heart or CNS disease. Packaging cells for producing the rAAVs described herein are provided. Method of treatment by delivery of, and pharmaceutical compositions comprising, the engineered rAAVs described herein are also provided. Also provided are methods of manufacturing the rAAVs with the engineered capsids described herein.

The invention is illustrated by way of examples infra describing the construction of rAAV9 capsids engineered with amino acid substitutions and assaying of tissue distribution when administered to non-human primates.

### 3.1. Embodiments

1. A recombinant AAV capsid protein comprising one or more amino acid substitutions relative to the wild type or unengineered capsid protein, in which the rAAV capsid protein is an AAV9 capsid protein (SEQ ID NO:74) with S263G/S269R/A273T substitutions, a G266A substitution, an N272A substitution, a W503R substitution, a Q474A substitution, 496-NNN/AAA-498 substitutions, has an insertion of the peptide TLAAPFK (SEQ ID NO:1) between Q588 and A589, S268 and S269, or S454 and G455, or an insertion of the peptide RTIGPSV (SEQ ID NO:12) between S454 and G455, or is an AAV8 capsid (SEQ ID NO:73) with an A269S substitution or 498-NNN/AAA-500 substitutions, or corresponding substitutions or peptide insertions in a capsid protein of another AAV type capsid.
2. The recombinant AAV capsid protein of embodiment 1 further comprising 498-NNN/AAA-500 amino acid substitutions for an AAV8 capsid protein (SEQ ID NO:73) or 496-NNN/AAA-498 amino acid substitutions for an AAV9 capsid protein (SEQ ID NO:74), or corresponding substitutions in a capsid protein of another AAV type capsid.
3. The recombinant AAV capsid protein of embodiments 1 or 2 which is an AAV8.BBB.LD capsid (SEQ ID NO:27), an AAV9.BBB.LD capsid (SEQ ID NO:29), an AAV9.496-NNN/AAA-498 capsid (SEQ ID NO:31), AAV9.496-NNN/AAA-498.W503R capsid (SEQ ID NO:32), AAV9.W503R capsid (SEQ ID NO:33), an AAV9.S454-TFR3 capsid (SEQ ID NO:42), AAV9.Q474A capsid (SEQ ID NO:34), AAV9.N272A.496-NNN/AAA-498 capsid (SEQ ID NO:49) or AAV9.N266A.496-NNN/AAA-498 capsid (SEQ ID NO:50).
4. The recombinant AAV capsid protein of embodiments 1 to 3 in which the amino acid substitutions or insertions are in an AAV9 capsid, including an AAV.PHP.eB capsid, protein, or an AAV8 capsid.
5. The recombinant AAV capsid protein of embodiment 1 or 2 wherein the AAV type capsid is AAV rh.34, AAV4, AAV5, AAV hu.26, AAV rh.31, AAV hu. 13, AAV hu.26, AAV hu. 56, AAV hu.53, AAV7, AAV rh. 10, AAV rh.64.R1, AAV rh.46 or AAV rh. 73.
6. The recombinant AAV capsid protein of any of embodiments 1 to 5, which when incorporated into a rAAV vector, the rAAV vector has increased targeting, transduction or genome integration into CNS cells, relative to a rAAV vector incorporating the corresponding wild type capsid protein without the amino acid substitutions or peptide insertions.
7. The recombinant capsid protein of embodiment 6, which when incorporated into a rAAV vector, the rAAV vector has decreased targeting, transduction or genome integration into liver cells, relative to a rAAV vector incorporating the corresponding wild type capsid protein without the amino acid substitutions or peptide insertions.
8. The recombinant capsid protein of embodiment 6 or 7, which when incorporated into a rAAV vector, the rAAV vector has decreased targeting, transduction or genome integration into dorsal root ganglion cells, relative to a rAAV vector incorporating the corresponding capsid protein without the amino acid substitutions or peptide insertions.
9. The recombinant capsid protein of any of the embodiments 6 to 8, which when incorporated into a rAAV vector, the rAAV vector has decreased targeting, transduction or genome integration into peripheral nerve cells, relative to a rAAV vector incorporating the corresponding capsid protein without the amino acid substitutions or peptide insertions.
10. The recombinant AAV capsid protein of any of embodiments 1 to 5, which when incorporated into a rAAV vector, the rAAV vector has increased targeting, transduction or genome integration into skeletal and/or cardiac muscle cells, relative to a rAAV vector incorporating the corresponding capsid protein without the amino acid substitutions or peptide insertions.
11. The recombinant capsid protein of embodiment 10, which when incorporated into a rAAV vector, the rAAV vector has decreased targeting, transduction or genome integration into liver cells, relative to a rAAV vector incorporating the corresponding capsid protein without the amino acid substitutions or peptide insertions.
12. The recombinant capsid protein of embodiment 10 or 11, which when incorporated into a rAAV vector, the rAAV vector has decreased targeting, transduction or genome integration into CNS cells, relative to a rAAV vector incorporating the corresponding capsid protein without the amino acid substitutions or peptide insertion.
13. The recombinant capsid protein of any of embodiments 10 to 12, which when incorporated into a rAAV vector, the rAAV vector has decreased targeting, transduction or genome integration into dorsal root ganglion cells, relative to an rAAV vector incorporating the corresponding capsid protein without the amino acid substitutions
14. A nucleic acid comprising a nucleotide sequence encoding the rAAV capsid protein of any of embodiments 1 to 13, or encoding an amino acid sequence sharing at least 80% identity therewith and retaining the biological activity of the capsid.
15. The nucleic acid of embodiment 14 encoding the rAAV capsid protein of any of embodiments 1 to 13.
16. A packaging cell capable of expressing the nucleic acid of embodiment 14 or 15 to produce AAV vectors comprising the capsid protein encoded by said nucleotide sequence.
17 ArAAV vector comprising the capsid protein of any of embodiments 1 to 13.
18. The rAAV vector of embodiment 17 further comprising a nucleic acid comprising transgene encoding a therapeutic protein or therapeutic nucleic acid operably linked to a regulatory sequence for expression in the muscle and/or CNS cells and flanked by AAV ITR sequences.
19. A pharmaceutical composition comprising the rAAV vector of embodiment 17 or 18 and a pharmaceutically acceptable carrier.
20. A method of delivering a transgene to a cell, said method comprising contacting said cell with the rAAV vector of embodiment 17 or 18, wherein said transgene is delivered to said cell.
21. The method of embodiment 20 in which the cell is a CNS cell, cardiac muscle cell or skeletal muscle cell.
22. A method of delivering a transgene to a target tissue of a subject in need thereof, said method comprising administering to said subject the rAAV vector of embodiment 17 or 18, wherein the transgene is delivered to said target tissue.
23. The method of embodiment 22 wherein the transgene is a muscle disease or heart disease therapeutic and said target tissue is cardiac muscle or skeletal muscle.
24. The method of embodiment 23, wherein the rAAV is administered systemically, including intravenously or intramuscularly.
25. The method of embodiment 22 wherein the transgene is a CNS disease therapeutic and said target tissue is CNS.
26. The method of embodiment 25 wherein the rAAV is administered intrathecally or intracerebroventricularly.
27. A pharmaceutical composition for use in delivering a transgene to a cell, said pharmaceutical composition comprising the rAAV vector of embodiment 17 or 18, wherein said transgene is delivered to said cell.
28. A pharmaceutical composition for use in delivering a transgene encoding a therapeutic protein or therapeutic nucleic acid to a target tissue of a subject in need thereof, said pharmaceutical composition comprising the rAAV vector of embodiment 17 or 18, wherein the transgene is delivered to said target tissue.
29. The pharmaceutical composition of embodiment 27 or 28 wherein said therapeutic protein or therapeutic nucleic acid is a muscle disease therapeutic or a heart disease therapeutic and said target tissue is cardiac muscle or skeletal muscle.
30. The pharmaceutical composition of embodiment 27 to 29 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in cardiac muscle or skeletal muscle cells compared to a reference AAV capsid.
31. The pharmaceutical composition of embodiment 27 to 30 wherein the rAAV exhibits at least 50%, 60%, 70%, 80%, 90%, 95% or 99% less transduction in liver compared to the reference AAV capsid.
32. The pharmaceutical composition of embodiment 27 to 31 wherein the rAAV exhibits at least 50%, 60%, 70%, 80%, 90%, 95% or 99% less transduction in dorsal root ganglion cells compared to the reference AAV capsid.
33. The pharmaceutical composition of embodiment 27, 28 or 32 wherein said therapeutic protein or nucleic acid is a CNS disease therapeutic and said target tissue is CNS.
34. The pharmaceutical composition of embodiment 27, 28, 32 or 33 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction and/or transgene transcription in CNS cells compared to a reference AAV capsid.
35. The pharmaceutical composition of embodiment 27, 28, 33 to 34 wherein the rAAV exhibits at least 50%, 60%, 70%, 80%, 90%, 95% or 99% less transduction in liver compared to the reference AAV capsid.
36. The pharmaceutical composition of embodiment 27, 28, 32 to 35 wherein the rAAV exhibits at least 50%, 60%, 70%, 80%, 90%, 95% or 99% less transduction in dorsal root ganglion cells compared to the reference AAV capsid.
37. The pharmaceutical composition of embodiments 27 to 36, wherein the AAV reference capsid is AAV8 or AAV9.
38. A method of treating a CNS disorder in a subject in need thereof, said method comprising administering a therapeutically effective amount of pharmaceutical composition of any of embodiments 27, 28, 32 to 37.
39. A method of or pharmaceutical composition for use in treating a muscle disorder in a subject in need thereof, said method or use comprising administering a therapeutically effective amount of the pharmaceutical composition of any of embodiments 27-31 and 37.
40. A method of or pharmaceutical composition for use in treating a subject diagnosed with a neurological disorder, said method or use comprising administering a therapeutically effective amount of an rAAV composition to the striatum of the subject by intraparenchymal administration or ICV administration wherein the rAAV composition comprises a modified AAV9 capsid packaging a transgene suitable for treating the neurological disorder, and the rAAV exhibits tropism for dopaminergic neurons.
41. The method or composition of embodiment 40, wherein the rAAV exhibits retrograde or anterograde transport to the substantia nigra.
42. A method of or pharmaceutical composition for use in delivery of a therapeutic product to dopaminergic neurons, said method or use comprising administering a rAAV composition into a brain region of a subject, wherein the region is innervated by dopaminergic neurons, and the rAAV composition expresses the therapeutic product in the neurons.
43. A method of or pharmaceutical composition for use in delivery of a therapeutic product to the substantia nigra, said method or use comprising administering an rAAV composition into a brain region of a subject in need thereof, wherein the region is innervated by dopaminergic neurons, and the rAAV composition expresses the therapeutic product in the substantia nigra.
44. The method or composition of any of embodiments 40 to 43, wherein the rAAV comprises a modified AAV9 capsid having a peptide insertion.
45. The method or composition of any of embodiments 40 to 44, wherein the rAAV comprises a modified AAV9 capsid having a peptide insertion in variable region IV.
46. The method or composition of any of embodiments 40 to 45 wherein the peptide is a TFR3 peptide RTIGPSV (SEQ ID NO:19).
47. The method or composition of any of embodiments 40 to 46 wherein the peptide is inserted after position S454 of the AAV9 capsid (SEQ ID NO:74).
48. The method or composition of any of embodiments 40 to 47 wherein the capsid is AAV9.S454-TFR3 (SEQ ID NO:42).
49. The method or composition of embodiment 48 in which the level of transgene mRNA in the neurons is 2 to 10 fold greater than in the neurons of a subject intraparenchymally administered a reference rAAV vector having a wild type AAV9 capsid.
50. The method or composition of embodiments 40 to 49 wherein the neurological disorder is a movement disorder.
51. The method or composition of embodiment 50 wherein the neurological disorder is Parkinson's disease, levodopa-induced dyskinesia (LID) in Parkinson's Disease (PD-LID) or tardive dyskinesia.
52. The method or composition of embodiment 40 wherein the neurological disorder is schizophrenia.
53. A method for or pharmaceutical composition for use in treating or ameliorating one or more symptoms of Parkinson's disease and/or levodopa-induced dyskinesia (LID) in Parkinson's Disease (PD-LID) in a patient in need thereof, said method or use comprising administering a therapeutically effective amount of a pharmaceutical composition comprising a rAAV having or comprising an AAV9.S454.Tfr3 capsid and an artificial genome comprising a transgene encoding a therapeutic protein or therapeutic nucleic acid effective to treat or ameliorate one or more symptoms of Parkinson's disease and/or levodopa-induced dyskinesia (LID) in Parkinson's Disease (PD-LID) in the patient, operably linked to a regulatory sequence that promotes expression in CNS cells and flanked by AAV ITR sequences.
54. The method or composition of embodiment 53, wherein the administration is intraparenchymal, ICV or to a brain region innervated by dopaminergic neurons.

### 4. BRIEF DESCRIPTION OF THE FIGURES

FIG. 1 depicts sequence comparison of the capsid amino acid sequences including the VR-IV loop of the adeno-associated virus type 9 (AAV9 VR-IV) from residues L447 to R476, (with residues 451-459 bracketed) to corresponding to regions of other AAVs. Figure discloses SEQ ID NOS: 53-62, respectively, in order of appearance. The top sequence is the consensus sequence, SEQ ID NO:52.

FIG. 2 depicts a protein model of an AAV capsid structure, showing capsid variable regions VR-IV, VR-V and VR-VIII. The box highlights the loop region of VR-IV which provides surface-exposed amino acids as represented in the model.

FIG. 3 depicts high packaging efficiency (titer) in terms of genome copies per mL (GC/mL) of wild type AAV9 and eight (8) candidate modified rAAV9 vectors (1090, 1091, 1092, 1093, 1094, 1095, 1096, and 1097), where the candidate vectors each contain a FLAG insert immediately after different sites within AAV9s VR-IV, from residues 1451 to Q458, respectively. All vectors were packaged with luciferase transgene in 10 mL culture; error bars represent standard error of the mean.

FIG. 4 demonstrates surface exposure of 1 VR-IV loop FLAG inserts in each of eight (8) candidate modified rAAV9 vectors (1090, 1091, 1092, 1093, 1094, 1095, 1096, and 1097), confirmed by immunoprecipitation of packaged vectors by binding to anti-FLAG resin.

FIGS. 5A-5B depict transduction efficiency in Lec2 cells, transduced with capsid vectors carrying the luciferase gene (as a transgene), which were packaged into either wild type AAV9 (9-luc), or into each of eight (8) candidate modified (FLAG peptide inserted) rAAV9 vectors (1090, 1091, 1092, 1093, 1094, 1095, 1096, and 1097); transduction activity is expressed as percent luciferase activity, taking the activity of 9-luc as 100% (FIG. 5A), or as Relative Light Units (RLU) per microgram of protein (FIG. 5B).

FIGS. 6A-6E. FIG. 6A depicts a bar graph illustrating that insertions immediately after S454 of AAV9 of varying peptide length and composition may affect production efficiencies of AAV particles in a packaging cell. Ten peptides of varying composition and length were inserted after S454 within AAV9 VR-IV. qPCR was performed on harvested supernatant of transfected suspension HEK293 cells five days post-transfection. The results depicted in the bar graph demonstrate that the nature of the insertions affects the ability of AAV particles to be produced and secreted by HEK293 cells, and indicated by overall yields (titer). (Error bars represent standard error of the mean length of peptide, which is noted on the Y-axis in parenthesis.) FIGS. 6B-6E depict fluorescence images of transduced cell cultures of the following cell lines: (6B) Lec2 cell line (6C) HT-22 cell line, (6D) hCMEC/D3 cell line, and (6E) C2C12 cell line. AAV9 wild type and S454 insertion homing peptide capsids containing GFP transgene were used to transduce the noted cell lines. P1 vector was not included in images due to extremely low transduction efficiency, and P8 vector was not included due to low titer. AAV9.S454.FLAG showed low transduction levels in every cell type tested.

FIG. 7 depicts alignment of AAVs 1-9e, 3B, rh10, rh20, rh39, rh73, rh74 version 1 and version 2, hu12, hu21, hu26, hu37, hu51 and hu53 capsid sequences with insertion sites for heterologous peptides after the initiation codon of VP2, and within or near variable region 1 (VR-I), variable region 4 (VR-IV), and variable region 8 (VR-VIII), all highlighted in grey; a particular insertion site within variable region eight (VR-VIII) of each capsid protein is shown by the symbol “#” (after amino acid residue 588 according to the amino acid numbering of AAV9).

FIGS. 8A-8C show T1 images following injection into three subjects (monkeys) (Prohance contrast agent was added to injected test article). Images A), B), and C) show each individual subject and location of the injection into the striatum for that subject.

FIG. 9 depicts qPCR analysis which revealed high copy number (RNA cp/μg) of total vector transcripts localized to putamen, caudate, GPe and interestingly, SNc, as well as other tissues (RNA copy number/ug, adjusted to log scale).

FIG. 10 depicts on overview of the relationship between vector spread from posterior compartments to anterior compartments, as an absolute measure of RNA copy number vs. log scale. Localization was observed in the putamen, as well as the caudate regions. Vector expression was strongest in injection site (e.g. putamen anterior).

FIGS. 11A and 11B depict absolute RNA copy number/ug distribution by qPCR (A) and results adjusted to log scale (B), including peripheral tissues. Expression in the periphery and in unrelated brain regions was negligible, which comports with known lack of anatomical connectivity from cerebellum or hippocampus to putamen.

FIG. 12 depicts NGS analysis to identify vector DNA from the capsid pool in putamen. The BC029 barcode indicates the mutated AAV9 capsid, AAV9.S454.Tfr3 is the highest transduced capsid identified in the putamen ipsilateral (relative to the location of injection) of this particular injected subject.

FIG. 13 depicts RNA abundance adjusted to input (normalized to 1 per ug). qPCR was performed to identify vector transcripts relative to high expressing capsids in the selected tissues: putamen ipsilateral (sample “punch” 1 or 2), caudate ipsilateral (punch 1 or 2), extreme capsule ipsilateral, golubus pallidus (external) (GPe) ipsilateral, amygdala ipsilateral, substantia nigra ipsilateral. AAV9.S454.Tfr3 demonstrates improved transduction relative to AAV9 following intraparenchymal delivery, for example a 4 to 5 fold increase RNA expression compared to AAV9 is observed in most brain regions analyzed.

FIGS. 14A and 14B show biodistribution of AAV9 and AAV9.454. Tfr3 vector RNA and DNA following IV administration of pooled capsids intravenously in NHPs.

FIGS. 15A and 15B illustrate the biodistribution of AAV9 and AAV9.454.Tfr3 vector RNA and DNA in selected tissues following IV administration of pooled capsids intravenously in mice.

FIGS. 16A and 16B illustrate the correlation between DNA abundances (FIG. 16A) and RNA abundances (FIG. 16B) in NHP and mouse at the injection site following intraparenchymal delivery to striatum (mouse) or putamen (NHP) of a barcoded library of rAAV having modified capsids. AAV9.S454.Tfr3 (BCO29) identified as top hit in NHP and mouse.

FIG. 17 illustrates AAV9 and AAV9.S454. Tfr3 (BC029) RNA levels (FIG. 17A) and AAV9 and AAV9.S454.Tfr3 (BC029) DNA levels (FIG. 17B) in NHP following intraparenchymal administration.

FIG. 18 illustrates AAV9 and AAV9.S454 TFR3 (BC029) RNA levels (FIG. 18A) and AAV9 and AAV9.S454 TFR3 (BC029) DNA levels (FIG. 18B) following intraparenchymal administration in mouse.

FIGS. 19A and 19B depict genome copy per ug DNA (left y-axis) and genome copy of BC029 (AAV9.S454. Tfr3 capsid) per ug DNA relative to genome copy per ug DNA AAV9 (right y-axis) in putamen (FIG. 19A) and caudate (FIG. 19B) in NHP administered PAVE118 library delivered by MRI-guided injection to the putamen.

FIGS. 20A and 20B depict RNA transcripts from BC029 (AAV9.S454.Tfr3 capsid) (left y-axis) and RNA transcripts from BC029 relative to RNA transcripts from AAV9 (right y-axis) in putamen (FIG. 20A) and caudate (FIG. 20B) in NHP administered PAVE118 library delivered by MRI-guided injection to the putamen.

FIGS. 21A and 21B show the ratio of RNA (transcript): DNA (genome copy) of BC029 (AAV9.S454.Tfr3 capsid) (FIG. 21A) and the ratio of RNA: DNA of BC029 relative to the RNA: DNA ratio of AAV9 (FIG. 21B) in putamen (solid bars) and caudate (hatched bars) in NHP administered PAVE118 library delivered by MRI-guided injection to the putamen.

FIGS. 22A and 22B show genome copies (GC/ug DNA) of BC029 (AAV9.S454.Tfr3 capsid) relative to AAV9 (FIG. 22A) and transcript copies from BC029 relative to AAV9 (FIG. 22B) in punch samples from the NHP central nervous system (substantia nigra 1, substantia nigra 2, rostral intralaminar thalamus, caudal intralaminar thalamus and frontal cortex) from NHP administered the PAVE118 library to the putamen.

FIGS. 23A-23C show GC/cell of BC029 (AAV9.S454.Tfr3 capsid) (left y-axis) and GC/cell BC029 DNA relative to GC/cell AAV9 (right y-axis) in the striatum (FIG. 23A), thalamus (FIG. 23B), and frontal cortex (FIG. 23C) of mice administered PAVE118 library by injection into the striatum.

FIGS. 24A-24C show transcripts/ug RNA from BC029 (AAV9.S454.Tfr3 capsid) (left y-axis) and transcripts/ug RNA BC029 relative to AAV9 (right y-axis) in the striatum (FIG. 24A), thalamus (FIG. 24B), and frontal cortex (FIG. 24C) of mice administered PAVE118 library by injection into the striatum.

FIGS. 25A and 25B show RNA: DNA ratio (transcripts/ug RNA per GC/cell DNA) for BC029 (FIG. 25A) and RNA: DNA ratio for BC029 relative to AAV9 (transcripts/ug RNA per GC/cell DNA) (FIG. 25B) in the striatum (solid bars), thalamus (hatched bars) and frontal cortex (checked bars) in mice administered PAVE118 library by injection into the striatum.

FIGS. 26A-26B show relative abundance of genome copy (FIG. 26A) and transcript copy (FIG. 26B) of AAV9 (light bars) and BC029 (dark bars) in CNS (frontal cortex, striatum and hippocampus) in mice administered PAVE118 library by ICV infusion to the left ventricle. FIG. 26C shows “off target” biodistribution after ICV administration (GC/ug DNA) of AAV9 (light bars) and BC029 (dark bars) in mouse liver, NHP liver, NHP heart, NHP lumbar DRG and NHP thoracic DRG.

### 5. DETAILED DESCRIPTION

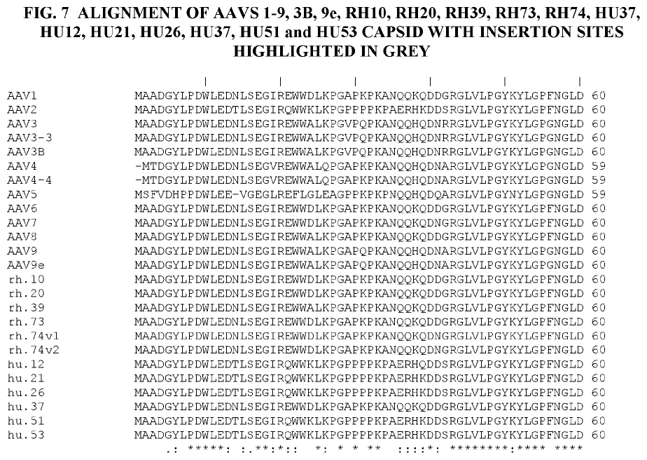

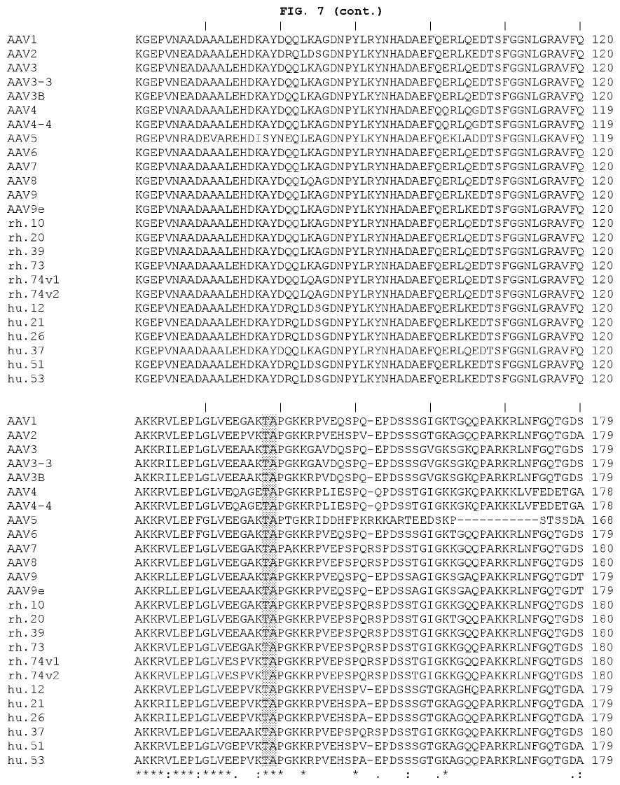

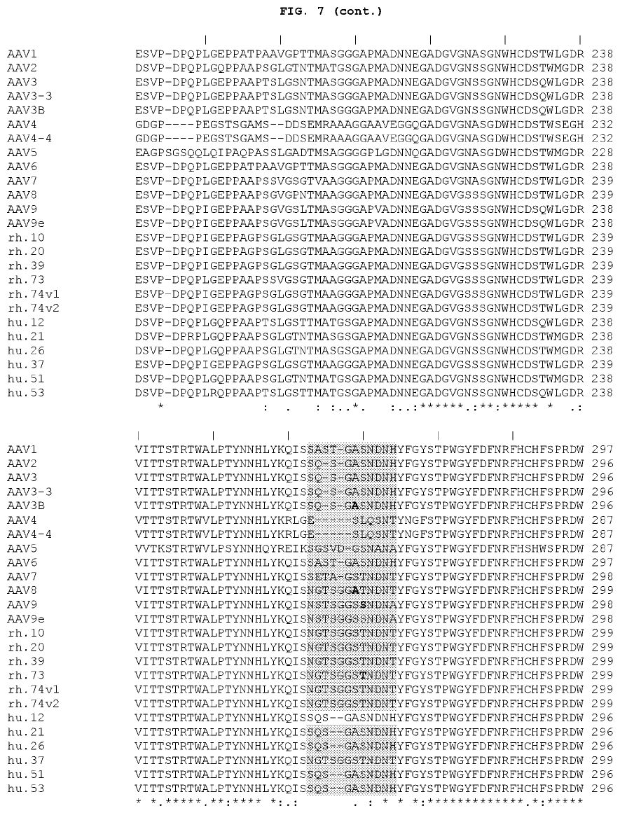

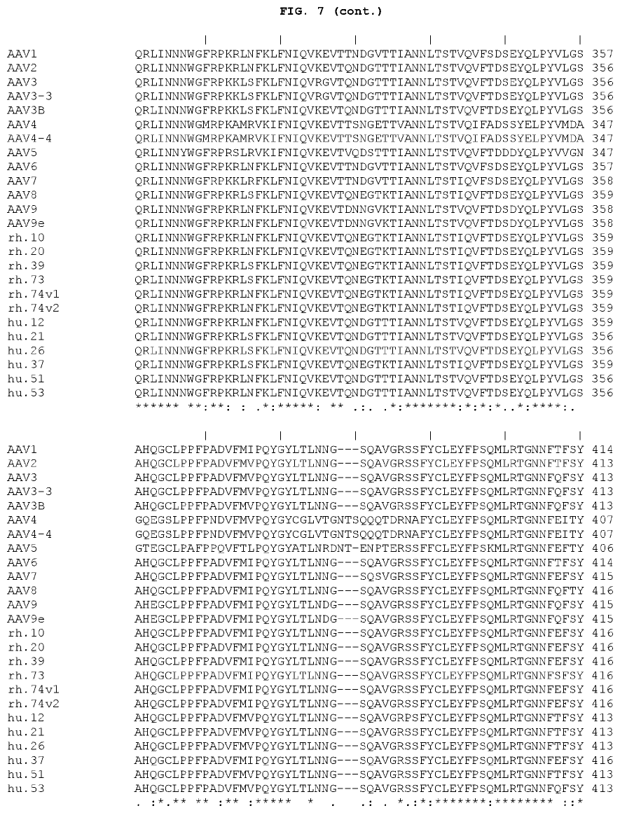

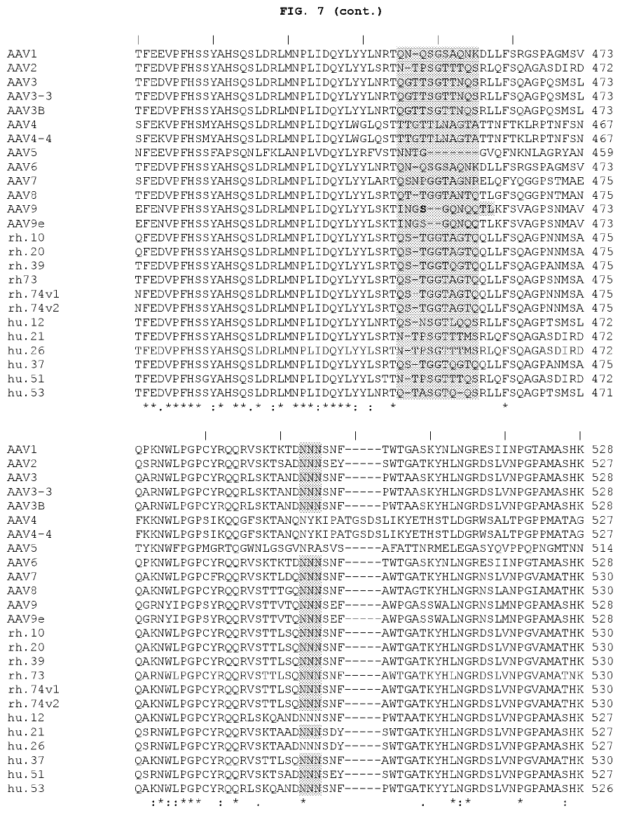

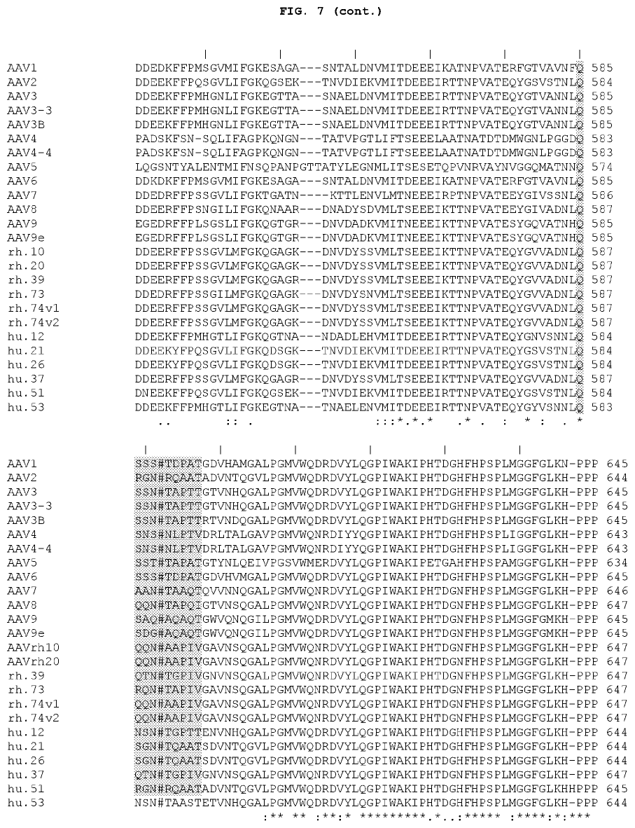

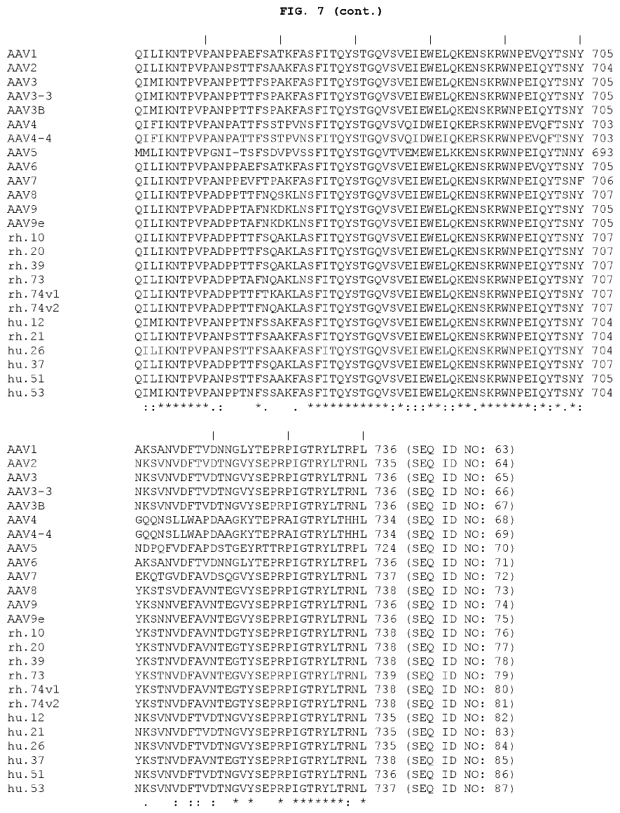

Provided are recombinant adeno-associated viruses (rAAVs) having capsid proteins engineered relative to a reference capsid protein, such that the rAAV has enhance desired properties, such as increased tissue targeting, including transduction, genome integration and transgene expression, particularly, preferentially, relative to the reference capsid protein (e.g., the unengineered or wild type capsid), to CNS, including dopaminergic neurons, or to heart and/or skeletal muscle tissue. In embodiments, the engineered capsid has reduced tropism (i.e., tissue targeting, transduction and integration of the rAAV genome) relative to the reference capsid for liver, dorsal root ganglion and/or peripheral nervous tissue to reduce toxicity of the AAV gene therapy. The modifications include amino acid substitutions (including 1, 2, 3, 4, 5, 6, 7 or 8 amino acid substitutions) and/or peptide insertions (4 to 20, or 7 contiguous amino acids, and in embodiments no more than 12 contiguous amino acids from a heterologous protein) as described herein. The AAV capsid protein to be engineered is, in certain embodiments, an AAV9 capsid protein or an AAV8 capsid protein. In other embodiments, the AAV capsid to be engineered is an AAV rh.34, AAV4, AAV5, AAV hu.26, AAV rh.31, AAV hu.13, AAV hu.56, AAV hu.53, AAV7, AAV rh64R1, AAV rh46 or AAV rh73 capsid protein. (See FIG. 7 and Table 17 for sequences)

Accordingly provided are engineered capsids having one or more amino acid substitutions that promote transduction and/or tissue tropism, particularly for enhanced, relative to an unengineered capsid, targeting for heart and/or skeletal muscle and, in embodiments, reduced, relative to an unengineered capsid, targeting for liver, dorsal root ganglion, and/or peripheral nervous tissue. Thus, injection of one AAV serotype into the striatum will be preferentially transported to e.g. the cortex, and a different AAV serotype injected into the striatum will be preferentially transported to e.g. the substantia nigra. Numerous other specificities were discovered beyond these representative brain regions, such as amygdala. In embodiments, the amino acid substitutions are S263F/S269T/A273T of AAV9, and corresponding substitutions in other AAV type capsids (for example according to the alignment in FIG. 7), or W530R, Q474A, N272A, or G266A of AAV9, and corresponding substitutions in other AAV type capsids or A269S of AAV8 and corresponding substitutions in other AAV capsids (for example, according to the alignment in FIG. 7). Also provided are capsids, particularly AAV9 capsids having a peptide TLAAPFK (SEQ ID NO:1) inserted between Q588 and A589 (herein PHP.hDYN) or alternatively between S268 and S269 or between S454 and G455) or inserted in another AAV capsid at a corresponding position (see, e.g., FIG. 7). Also provided are capsids, particularly AAV9 capsids having a peptide RTIGPSV (SEQ ID NO: 19) inserted between S454 and G455 (herein AAV9.S454-TFR3; SEQ ID NO:42) or alternatively between S268 and S269 or between Q588 and A589) or inserted in another AAV capsid at a corresponding position (see, e.g., FIG. 7), which capsids have a tropism and exhibit increased transcriptional activity in the putamen and caudate, including for dopaminergic neurons and exhibit retrograde or anterograde transport to the substantia nigra, and to the rostral intralaminar thalamus, caudal intralaminar thalamus and frontal cortex including when administered to the striatum. Also provided are capsids, particularly AAV9 capsids having S263G/S269T/A273T, and 496-NNN/AAA-498 substitutions in the amino acid sequence of AAV9 (herein AAV9.BBB.LD; SEQ ID NO:74) (see, e.g., FIG. 7), which capsids have a tropism for amygdala and/or cortex and exhibit retrograde or anterograde transport to the amygdala and/or cortex, including when administered to the striatum. Or, alternatively, the capsid is an AAV9 PHP.eB capsid (which has the modifications A587D and Q588G and insertion of the peptide TLAVPFK (SEQ ID NO:20) between G588 and A589) and the peptide TILSRSTQTG (SEQ ID NO:15) between position 138 and 139, or the corresponding. Additional capsids have a Kidney 1 peptide LPVAS (SEQ ID NO:6) inserted into the capsid, for example between S454 and G455 of AAV9, or alternatively between S268 and S269 or between Q588 and A589, or the corresponding position of a different capsid. In some embodiments, the capsids can comprise R697W substitution of AAV rh64R1. The capsids having these amino acid substitutions and insertions may further have substitutions of the NNN (asparagines) at 496 to 498 with AAA (alanines) of the AAV9 capsid or at positions 498 to 500 of the AAV8 capsid, or corresponding substitutions in other AAV type capsids. Engineered capsids include AAV8.BB.LD (A269S,498-NNN/AAA-500 substitutions in the amino acid sequence of AAV8, SEQ ID NO 66), AAV9.BB.LD (S263G/S269T/A273T, 496-NNN/AAA-498 substitutions in the amino acid sequence of AAV9, SEQ ID NO 67), AAV9.BBB (SEQ ID NO: 28), AAV9.496-NNN/AAA-498 (SEQ ID NO:31), AAV9.496-NNN/AAA-498. W503R (SEQ ID NO:32), AAV9.W503R (SEQ ID NO:33), AAV9.Q474A (SEQ ID NO:34), AAV9.S454. Tfrl (SEQ ID NO:41) or AAV9.S454-TFR3 (SEQ ID NO:42). In other examples, the capsid can be AAV9.N272A.496-NNN-498 (SEQ ID NO:49) or AAV9.G266A.496-NNN-498 (SEQ ID NO:50). In other embodiments, the capsid is not an engineered capsid, but is an AAV7 (SEQ ID NO:72), AAVrh. 10 capsid (SEQ ID NO:76), an AAVrh.46 capsid (SEQ ID NO:97), an AAVrh.64.R1 capsid (SEQ ID NO:48) or an AAVrh.73 capsid (SEQ ID NO:79). In certain embodiments, transduction is measured by detection of transgene, such as GFP fluorescence. The capsids having these amino acid substitutions and insertions may further have substitutions of the NNN (asparagines) at 496 to 498 with AAA (alanines) of the AAV9 capsid, or corresponding substitutions in other AAV type capsids. This engineered capsid may exhibit preferential targeting for heart and/or skeletal muscle, and reduced targeting (compared to an AAV bearing the unengineered capsid) for liver and/or dorsal root ganglion cells and/or peripheral nervous system tissue, and may particularly useful for delivery of a transgene encoding a therapeutic protein or nucleic acid for treatment of a muscle disease.

In another embodiment, provided is a recombinant capsid protein, including an engineered AAV9 capsid protein, and an rAAV comprising the capsid protein, in which the peptide TLAVPFK (SEQ ID NO:20) is inserted between G588 and A589 of AAV9, and, in particular, the capsid protein also has amino acid substitutions A587D/Q588G (PHP.eB) and further has the peptide TILSRSTQTG (SEQ ID NO: 15) inserted after position 138 of AAV9 (collectively, AAVPHPeB.VP2Herp; see Table 17), or in the corresponding positions of another AAV. Additional capsids have a Kidneyl peptide LPVAS (SEQ ID NO:6) (or alternatively CLPVASC (SEQ ID NO:5)) inserted into the capsid, for example between S454 and G455 of AAV9 (see Table 17), or alternatively between S268 and S269 or between Q588 and A589, or the corresponding position of a different capsid. Such an engineered capsid may exhibit preferential targeting for heart and skeletal muscle, and reduced targeting (as compared to an AAV having the unengineered capsid) for liver and/or dorsal root ganglion cells and may particularly useful for delivery of a transgene encoding a therapeutic protein or nucleic acid for treatment of a muscle disease (such as, but not limited to a muscular dystrophy).

In embodiments the engineered rAAV exhibits at least 1.1-fold, 1.5-fold, 2-fold, 3-fold, 4-fold, 5-fold, 6-fold, 7-fold, 8-fold, 9-fold, or 10-fold greater transduction in cardiac muscle and/or skeletal muscle cells compared to a reference AAV capsid, including an AAV9 capsid or an AAV8 capsid, or the parental capsid. In particular embodiments, the muscle is gastrocnemius muscle, bicep, tricep and/or heart muscle. In further embodiments, the engineered rAAV exhibits of 50%, 60%, 70%, 80%, 90%, 95% or 99% less transduction in liver compared to the reference AAV capsid compared to a reference AAV capsid, including an AAV9 capsid or an AAV8 capsid, or the parental capsid. In further embodiments, the rAAV exhibits of 50%, 60%, 70%, 80%, 90%, 95% or 99% less transduction in dorsal root ganglion cells (including in cervical, thoracic or lumbar DRG cells) compared to the reference AAV capsid. The enhanced and/or reduce transduction may be with any mode of administration, by intravenous administration, intramuscular administration, or any type of systemic administration, intrathecal administration or ICV administration.

Also provided are engineered capsids having one or more amino acid substitutions that promote transduction and/or tissue tropism, particularly for enhanced, relative to an unengineered capsid, targeting for CNS and, in embodiments, reduced, relative to an unengineered capsid, targeting for liver, dorsal root ganglion, and/or peripheral nervous tissue. In embodiments, the amino acid substitutions areA269S of AAV8 (or at a corresponding position in a different AAV serotype capsid), S263G/S269T/A273T of AAV9 (or at a qcorresponding position in a different AAV serotype capsid), N272A or N266A of AAV9 (or at a corresponding position in a different AAV serotype capsid), Q474A of AAV9 (or at a corresponding position in a different AAV serotype capsid), or W503R of AAV9 (or at a corresponding position in a different AAV serotype capsid), or R697W of rh64R1 (or at a corresponding position in a different AAV serotype capsid) or an insertion of the peptide RTIGPSV (SEQ ID NO:19) after S454 of AAV9 (or at a corresponding position in a different AAV serotype capsid). The capsids having these amino acid substitutions and insertions may further have or alternatively have substitutions of the NNN (asparagines) at 496 to 498 with AAA (alanines) of the AAV9 capsid (SEQ ID NO:74) or have substitutions of the NNN (asparagines) at 498 to 500 with AAA (alanines) of the AAV8 capsid (SEQ ID NO:73), or corresponding substitutions in other AAV type capsids. This engineered capsid may exhibit preferential targeting for CNS, and reduced targeting (compared to an AAV bearing the unengineered capsid) for liver and/or dorsal root ganglion cells and/or peripheral nervous system tissue, and may particularly useful for delivery of a transgene encoding a therapeutic protein or nucleic acid for treatment of a CNS disease.

Also provided are recombinant capsid proteins, and rAAVs comprising them, that have inserted peptides that target and/or promote rAAV cellular uptake, transduction and/or genome integration in CNS tissue and, in embodiments, reduced, relative to an unengineered capsid, targeting for liver, dorsal root ganglion, and/or peripheral nervous tissue, for example, the peptide RTIGPSV (SEQ ID NO:19), TILSRSTQTG (SEQ ID NO:15); TLAVPFK (SEQ ID NO: 20); or TLAAPFK (SEQ ID NO:1). In particular embodiments, the peptide RTIGPSV is inserted between S454 and G455 of AAV9 (see FIG. 7, AAV9S454-TFR3, SEQ ID NO:42; Table 17). In particular embodiments the peptide TLAAPFK (SEQ ID NO:1) is inserted between Q588 and A589 of AAV9 (AAV9.hDyn; see Table 17), or the corresponding position of another AAV (see FIG. 7). Alternatively, the capsid is rh.34, rh. 10, rh.46, rh. 73, or rh64. R1 (FIG. 7 or Table 17 for sequence), or an engineered form of rh.34, rh.10, rh.46, rh.73, or rh64.R1. These engineered capsids may exhibit preferential targeting for CNS, and reduced targeting (compared to an AAV bearing the unengineered capsid) for liver and/or dorsal root ganglion cells and/or peripheral nervous system tissue, and may particularly useful for delivery of a transgene encoding a therapeutic protein or nucleic acid for treatment of a CNS disease. In embodiments, the engineered capsids, particularly capsids with the TFR3 peptide (RTIGPSV (SEQ ID NO:19) inserted, such as AAV9.S454-TFR3 capsid) may exhibit preferential targeting for areas of the CNS, including when locally administered to the striatum (or intracerebroventricularly), such as dopaminergic neurons. These capsids may exhibit retrograde or anterograde transport to the substantia nigra and may target the caudate and external globus pallidus. In embodiments, AAV9.S454-Tfr3 capsids exhibit, upon intraparenchymal or intracerebroventricular administration, enhanced transduction efficiency and transcriptional activity compared to, for example, AAV9 (may be 2 fold, 4 fold, 5 fold, 8 fold or 10 fold better) in the putamen and/or caudate, and may include regions of interest, including the substantia nigra, intralaminar thalamus, and frontal cortex. In embodiments, the capsids further exhibit reduced targeting (compared to an AAV bearing the unengineered capsid) for liver and/or dorsal root ganglion cells and/or peripheral nervous system tissue, and may particularly useful for delivery of a transgene encoding a therapeutic protein or therapeutic nucleic acid for treatment of a CNS disease, particularly a disease involving dopaminergic neurons, including movement disorders, such as Parkinson's Disease, levodopa-induced dyskinesia (LID) in Parkinson's Disease (PD-LID) and tardive dyskinesia, or alternatively, schizophrenia.

In embodiments the engineered rAAV exhibits at least 1.1-fold, 1.5-fold, 2-fold, 3-fold, 4-fold, 5-fold, 6-fold, 7-fold, 8-fold, 9-fold, or 10-fold greater transduction or transgene transcription (mRNA abundance) or transgene expression in CNS tissue compared to a reference AAV capsid, such as the parental capsid or AAV8 or AAV9. The CNS tissue may be one or more of the frontal cortex, hippocampus, cerebellum, midbrain and/or hindbrain or, in alternative embodiments, may be dopaminergic neurons, putamen, caudate, substantia nigra, and/or globus pallidus (external). In further embodiments, the engineered rAAV exhibits of 50%, 60%, 70%, 80%, 90%, 95% or 99% less transduction in liver compared to the reference AAV capsid such as the parental capsid or AAV8 or AAV9. In further embodiments, the rAAV exhibits of 50%, 60%, 70%, 80%, 90%, 95% or 99% less transduction in dorsal root ganglion cells (including in cervical, thoracic or lumbar DRG cells) compared to the reference AAV capsid such as the parental capsid or AAV8 or AAV9. The enhanced and/or reduce transduction may be with any mode of administration, by intravenous administration, intramuscular administration, or any type of systemic administration, intrathecal administration or ICV administration. In embodiments, the administration is intraparenchymal administration, including delivery to the striatum.

Recombinant vectors comprising the capsid proteins also are provided, along with pharmaceutical compositions thereof, nucleic acids encoding the capsid proteins, and methods of making and using the capsid proteins and rAAV vectors having the engineered capsids for targeted delivery, improved transduction and/or treatment of disorders associated with the target tissue.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “subject”, “host”, and “patient” are used interchangeably. As used herein, a subject is a mammal such as a non-primate (e.g., cows, pigs, horses, cats, dogs, rats etc.) or a primate (e.g., monkey and human), or, in certain embodiments, a human.

As used herein, the terms “therapeutic agent” refers to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

As used herein, the term “prophylactic agent” refers to any agent which can be used in the prevention, delay, or slowing down of the progression of a disease or disorder, where the disease or disorder is associated with a function to be provided by a transgene. As used herein, a “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The “central nervous system” (“CNS”) as used herein refers to neural tissue reaches by a circulating agent after crossing a blood-brain barrier, and includes, for example, the brain, optic nerves, cranial nerves, and spinal cord. The CNS also includes the cerebrospinal fluid, which fills the central canal of the spinal cord as well as the ventricles of the brain.

Throughout the specification, AAV “serotype” refers to an AAV having an immunologically distinct capsid, a naturally-occurring capsid, or an engineered capsid.

### 5.2. Recombinant AAV Capsids and Vectors

Provided are recombinant adeno-associated viruses (rAAVs) having capsid proteins engineered relative to a reference capsid protein, such that the rAAV has enhance desired properties, such as increased tissue targeting, including transduction, genome integration and transgene expression, particularly, preferentially, relative to the reference capsid protein (e.g., the unengineered or wild type capsid), to CNS, including dopaminergic neurons, or to heart and/or skeletal muscle tissue. In embodiments, the engineered capsid has reduced tropism (i.e., tissue targeting, transduction and integration of the rAAV genome, transcription and/or expression of the transgene) relative to the reference capsid for liver, dorsal root ganglion and/or peripheral nervous tissue to reduce toxicity of the AAV gene therapy. The modifications include amino acid substitutions (including 1, 2, 3, 4, 5, 6, 7 or 8 amino acid substitutions) and/or peptide insertions (4 to 20, or 7 contiguous amino acids, and in embodiments no more than 12 contiguous amino acids from a heterologous protein) as described herein.

5.2.1 Engineered Capsids with Amino Acid Substitutions

In some embodiments, AAV capsids were modified by introducing selected single to multiple amino acid substitutions which increase effective gene delivery to the CNS or to cardiac or skeletal muscle, detarget the liver and/or dorsal root ganglion to reduce toxicity, and/or reduce immune responses of neutralizing antibodies.

In particular embodiments the capsids have one or more amino acid substitutions including a W503R substitution, a Q474 substitutional a N272A or N266A substitution in AAV9 or the corresponding substitution in another AAV serotype or an A269S substitution in AAV8 or the corresponding substitution in another AAV serotype. rAAV having a capsid with the Q474A substitution may be particularly useful for delivery to skeletal and/or cardiac muscle or CNS tissue and rAAV having a capsid with the W503R substitution may be particularly useful for delivery to CNS tissue, particularly with reduced, compared to reference capsid containing rAAVs, transduction in the liver and/or DRGs. Other substitutions include S263G/S269R/A273T substitutions in AAV9 or A587D/Q588G in AAV9 or corresponding substitutions in other AAV serotypes. In some embodiments, the rAAV capsid can have a R697W substitution. The capsids having these amino acid substitutions and insertions may further have substitutions of the NNN (asparagines) at 496 to 498 with AAA (alanines) of the AAV9 capsid, or of the NNN (asparagines) at 498 to 500 with AAA (alanines) of the AAV8 capsid corresponding substitutions in other AAV type capsids. Other AAV serotypes that may be used for the amino acid substitutions and that may be the reference capsid include AAV8, AAV rh.34, AAV4, AAV5, AAV hu.26, AAV rh.31, AAV hu. 13, AAV hu.26, AAV hu.56, AAV hu.53, AAV7, rh64R1, rh46 or rh73. In particular embodiments for CNS delivery, the capsid is rh34, either unmodified or serving as the parental capsid to be modified as detailed herein.

In embodiments, the capsid has an insertion of a TFR3 peptide (RTIGPSV, SEQ ID NO: 19), including in variable region IV (see FIG. 7) of a parental capsid, which may be AAV9. The peptide may be inserted between S454 and G455 of the AAV9 capsid sequence or the corresponding position of another capsid (See FIG. 7 for exemplary alignment). Capsids include AAV9S454-TFR3 (SEQ ID NO:42; see Table 17). Such capsids may have improved transduction efficiency and transcriptional activity (such as 2 fold, 4 fold, 6 fold, 8 fold or 10 fold) in CNS regions of interest relative to parental capsids such as AAV9, including when administered intraparenchymally or by ICV administration.

Effective gene delivery to the CNS by intravenously administered rAAV vectors requires crossing the blood brain barrier. Key clusters of residues on the AAVrh. 10 capsid that enabled transport across the brain vasculature and widespread neuronal transduction in mice have recently been reported. Specifically, AAVrh. 10-derived amino acids N262, G263, T264, S265, G267, S268, T269, and T273 were identified as key residues that promote crossing the BBB (Albright et al, 2018, Mapping the Structural Determinants Required for AAVrh.10 Transport across the Blood-Brain Barrier). Amino acid substitutions in capsids, such as AAV8 and AAV9 capsids that promote rAAV crossing of the blood brain barrier, transduction, detargeting of the liver and/or reduction in immune responses have been identified.

In some embodiments, provided are capsids having one or more amino acid substitutions that promote transduction and/or tissue tropism of the rAAV having the modified capsid. In particular embodiments, provided are capsids having a single mutation at amino acid 269 of the AAV8 capsid replacing alanine with serine (A269S) (see, Tables 5a-5c, herein referred to as AAV8.BBB) and amino acid substitutions at corresponding positions in other AAV types. In some embodiments, provided are capsids having multiple substitutions at amino acids 263, 269, and 273 of the AAV9 capsid resulting in the following substitutions: S263G, S269T, and A273T (herein referred to as AAV9.BBB) or substitutions corresponding to these positions in other AAV types.

Exposure to the AAV capsid can generate an immune response of neutralizing antibodies. One approach to overcome this response is to map the AAV-specific neutralizing epitopes and rationally design an AAV capsid able to evade neutralization. A monoclonal antibody, specific for intact AAV9 capsids, with high neutralizing titer has recently been described (Giles et al, 2018, Mapping an Adeno-associated Virus 9-Specific Neutralizing Epitope To Develop Next-Generation Gene Delivery Vectors). The epitope was mapped to the 3-fold axis of symmetry on the capsid, specifically to residues 496-NNN-498 and 588-QAQAQT-592 of AAV9 (SEQ ID NO:8). Capsid mutagenesis demonstrated that single amino acid substitution within this epitope markedly reduced binding and neutralization. In addition, in vivo studies showed that mutations in the epitope conferred a “liver-detargeting” phenotype to the mutant vectors, suggesting that the same residues are also responsible for AAV9 tropism. Liver detargeting has also been associated with substitution of amino acid 503 replacing tryptophan with arginine. Presence of the W503R mutation in the AAV9 capsid was associated with low glycan binding avidity (Shen et al, 2012, Glycan Binding Avidity Determines the Systemic Fate of Adeno-Associated Virus Type 9).

In some embodiments, provided are capsids in which the AAV8.BBB and AAV9.BBB capsids were further modified by substituting asparagines at amino acid positions 498, 499, and 500 of AAV8 (herein referred to as AAV8.BBB.LD) or 496, 497, and 498 of AAV9 (herein referred to as AAV9.BBB.LD) with alanines. In some embodiments, the AAVrh10 capsid was modified by substituting three asparagines at amino acid positions 498, 499, and 500 to alanines (AAVrh10.LD) (Tables 5a-5c).

In some embodiments, provided are capsids having three asparagines at amino acid positions 496, 497, and 498 of the AAV9 capsid replaced with alanines and also tryptophan at amino acid 503 of the AAV9 capsid with arginine or capsids with substitutions corresponding to these positions in other AAV types. In some embodiments, provided are capsids having glutamine at amino acid position 474 of the AAV9 capsid substituted with alanine or capsids with substitutions corresponding to this position in other AAV types.

In some embodiments, the capsid is an AAV8.BB.LD capsid (A269S,498-NNN/AAA-500 substitutions in the amino acid sequence of AAV8, SEQ ID NO 66), an AAV9.BBB.LD capsid (S263G/S269T/A273T, 496-NNN/AAA-498 substitutions in the amino acid sequence of AAV9, SEQ ID NO:74), an AAV9.496-NNN/AAA-498 capsid (SEQ ID NO: 31), an AAV9.496-NNN/AAA-498.W503R capsid (SEQ ID NO:32), an AAV9.W503R capsid (SEQ ID NO:33), or an AAV9.Q474A capsid (SEQ ID NO:34). In other examples, the capsid can be an AAV9.N272A.496-NNN-498 capsid (SEQ ID NO:49) or an AAV9.G266A.496-NNN-498 capsid (SEQ ID NO:50).

In some embodiments, the rAAVs described herein increase tissue-specific (such as, but not limited to, CNS or skeletal and/or cardiac muscle) cell transduction in a subject (a human, non-human-primate, or mouse subject) or in cell culture, compared to the rAAV not comprising the amino acid substitution. In some embodiments, the increase in tissue specific cell transduction (or transgene transcription or expression) is at least 2, 10, 20, 30, 40, 50, 60, 70, 80, 90, or 100 fold more than that without the modification. For example, in some embodiments, there is a 50-80 fold increase in tissue specific cell transduction compared to transduction with the same AAV type without the modification. The increase in transduction may be assessed using methods described in the Examples herein and known in the art.

In some embodiments, the rAAVs described herein increase the incorporation of rAAV genomes into a cell or tissue type in a subject (a human, non-human primate or mouse subject) or in cell culture to the rAAV not comprising the peptide insertion. In some embodiments, the increase in genome integration is at least 2, 10, 20, 30, 40, 50, 60, 70, 80, 90, or 100 fold more than an AAV having a capsid without the modification (i.e., the parental capsid). For example, in some embodiments, there is a 50-80 fold increase in genome integration compared to genome integration with the same AAV type without the modification.

5.2.2 rAAV Vectors with Peptide Insertions

Provided are rAAVs having capsid proteins with one or more (generally one or two) peptide insertions wherein the peptide insertion increase effective gene delivery to the CNS or to cardiac or skeletal muscle and to detarget the liver and/or dorsal root ganglion to reduce toxicity relative to the parental capsid protein. In particular embodiments, the peptides include RTIGPSV (SEQ ID NO:12), TLAVPFK (SEQ ID NO:20), TLAAPFK (SEQ ID NO:1), or TILSRSTQTG (SEQ ID NO:15) (or an at least 4, 5, 6, 7 amino acid portion thereof). The peptides may be inserted into the AAV9 capsid, for example after the positions 138; 262-273; 452-461; 585-593 of AAV9 cap, particularly after position 138, 454 or 588 of AAV9 or a corresponding position in another AAV as detailed herein. In particular embodiments, the capsid has the peptide TLAVPFK (SEQ ID NO:20) is inserted between G588 and A589 of AAV9, and, in particular, the capsid protein also has amino acid substitutions A587D/Q588G (PHP.eB) and further has the peptide TILSRSTQTG (SEQ ID NO:15) inserted after position 138 of AAV9 (collectively, AAVPHPeB.VP2Herp; see Table 17), or in the corresponding positions of another AAV. Additional capsids have a Kidney 1 peptide LPVAS (SEQ ID NO:6) inserted into the capsid, for example between 454 and 455 of AAV9 (see Table 17), or alternatively or alternatively between S268 and S269 or between Q588 and A589 of AAV9 or the corresponding position of another AAV serotype. Such an engineered capsid may exhibit preferential targeting for heart and skeletal muscle, and reduced targeting (as compared to an AAV having the unengineered capsid) for liver and/or dorsal root ganglion cells and may particularly useful for delivery of a transgene encoding a therapeutic protein or therapeutic nucleic acid for treatment of a muscle disease (such as, but not limited to a muscular dystrophy).

In some embodiments, the peptide insertion comprises at least 4, 5, 6, 7, 8, 9, or all 10 consecutive amino acids of sequence TILSRSTQTG (SEQ ID NO:15), preferably which contains the TQT or STQT (SEQ ID NO:9) motif. In some embodiments, the peptide insertion consists of at least 4, 5, 6, 7, 8, 9, or all 10 consecutive amino acids of sequence TILSRSTQTG (SEQ ID NO:15), preferably which contains the TQT or STQT (SEQ ID NO:9) motif.

In certain embodiments, the peptide insertion may be a sequence of consecutive amino acids from a domain that targets kidney tissue, or a conformation analog designed to mimic the three-dimensional structure of said domain. In some embodiments, the kidney-homing domain comprises the sequence CLPVASC (SEQ ID NO:5) (see, e.g., U.S. Pat. No. 5,622,699). In some embodiments, the peptide insertion from said kidney-homing domain comprises at least 4, 5, 6, or all 7 amino acids from sequence CLPVASC (SEQ ID NO:5). In some embodiments, the peptide insertion comprises or consists of the sequence CLPVASC (SEQ ID NO: 5).

It has been found that both of the cysteine residues in certain homing peptides can be deleted without significantly affecting the organ homing activity of the peptide. For example, a peptide having the sequence LPVAS (SEQ ID NO:6) also can be a kidney-homing peptide. Methods for determining the necessity of a cysteine residue or of amino acid residues N-terminal or C-terminal to a cysteine residue for organ homing activity of a peptide are routine and well known in the art. Thus, in some embodiments, the peptide insertion comprises at least 4 or all 5 amino acids from sequence LPVAS (SEQ ID NO:6). In some embodiments, the peptide insertion comprises or consists of the sequence LPVAS (SEQ ID NO:6).

In particular embodiments, provided are rAAVs having a capsid that has the peptide TLAAPFK (SEQ ID NO:1) is inserted between Q588 and A589 of AAV9 (AAV9.hDyn; see Table 17), or the corresponding position of another AAV (see, e.g., FIG. 7). Such an engineered capsid may exhibit preferential targeting for CNS tissue, and reduced targeting (as compared to an AAV having the unengineered capsid) for liver and/or dorsal root ganglion cells and may particularly useful for delivery of a transgene encoding a therapeutic protein or therapeutic nucleic acid for treatment of a CNS disease.

In embodiments, provided are rAAVs having a capsid with the peptide RTIGPSV (TFR3 peptide; SEQ ID NO:19). In embodiments, the RTIGPSV peptide may be inserted into the VR-IV loop of AAV9 or any other appropriate capsid. For example, it is inserted between S454 and G455 of AAV9 (SEQ ID NO:74; FIG. 7) and may be AAV9S454-TFR3 (SEQ ID NO: 42). Such capsids have a tropism for dopaminergic neurons (see Example 9), including when administered directly to the CNS, for example, by intraparenchymal administration to the striatum (or ICV administration), resulting in delivery, including by retrograde and/or anterograde transport, to other areas of the brain. Administration to the striatum of such capsids having the TFR3 peptide insert, such as AAV9S454-TFR3 provides delivery to dopaminergic neurons and regions of the brain including the substantia nigra, caudate, putamen, globus pallidus (external), intralaminar thalamus and frontal cortex. In embodiments, the AAV9S454-TFR3 has enhanced transcriptional activity and superior transduction efficiency (including 2 fold, 4 fold, 6 fold, 8 fold or 10 fold) in these CNS regions relative to the parental AAV9 capsid.

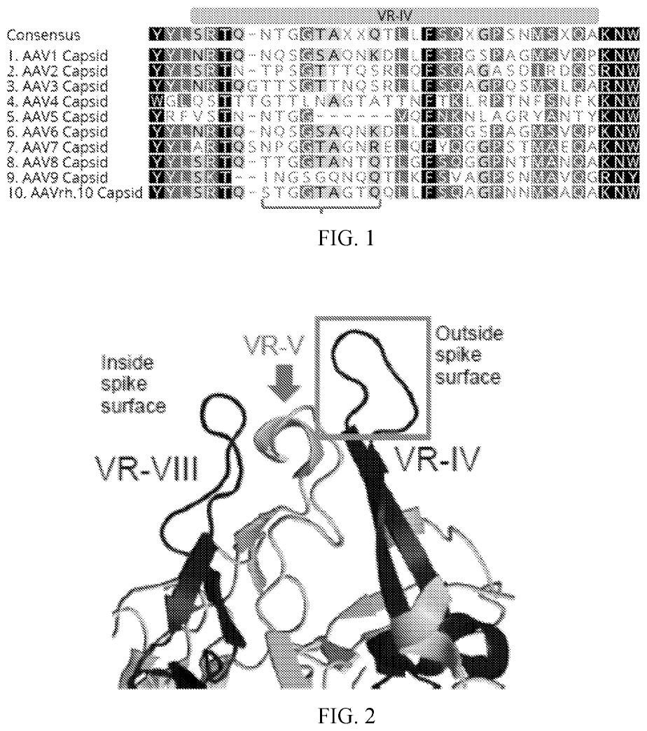

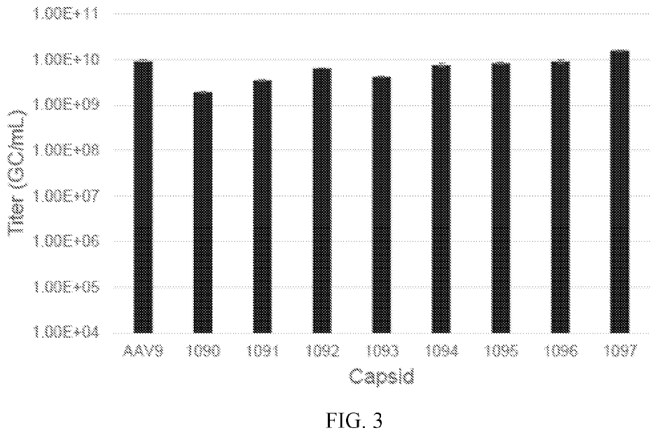

Provided are capsids with peptide insertions at positions amenable to peptide insertions within and near the AAV9 capsid VR-IV loop (see FIG. 2) and corresponding regions on the VR-IV loop of capsids of other AAV types. Though previous studies analyzed potential positions in various AAVs, none identified the AAV9 VR-IV as amenable for this purpose (consider, e.g., Wu et al, 2000, “Mutational Analysis of the Adeno-Associated Virus Type 2 (AAV2) Capsid Gene and Construction of AAV2 Vectors with Altered Tropism,” J of Virology 74 (18): 8635-8647; Lochrie et al, 2006, “Adeno-associated virus (AAV) capsid genes isolated from rat and mouse liver genomic DNA define two new AAV species distantly related to AAV-5,” Virology 353:68-82; Shi and Bartlett, 2003, “RGD Inclusion in VP3 Provides Adeno-Associated Virus Type 2 (AAV2)-Based Vectors with a Heparan Sulfate-Independent Cell Entry Mechanism,” Molecular Therapy 7 (4): 515525-; Nicklin et al., 2001, “Efficient and Selective AAV2-Mediated Gene Transfer Directed to Human Vascular Endothelial Cells” Molecular Therapy 4 (2): 174-181; Grifman et al., 2001, “Incorporation of Tumor-Targeting Peptides into Recombinant Adeno-associated Virus Capsids,” Molecular Therapy 3 (6): 964-975; Girod et al. 1999, “Genetic capsid modifications allow efficient re-targeting of adeno-associated virus type 2,” Nature Medicine 3 (9): 1052-1056; Douar et al., 2003, “Deleterious effect of peptide insertions in a permissive site of the AAV2 capsid, “Virology 309:203-208; and Ponnazhagan, et al. 2001, J. of Virology 75 (19): 9493-9501).

Accordingly, provided are rAAV vectors carrying peptide insertions at these points, in particular, within surface-exposed variable regions in the capsid coat, particularly within or near the variable region IV of the capsid protein. In some embodiments, the rAAV capsid protein comprises a peptide insertion immediately after (i.e., connected by a peptide bond C-terminal to) an amino acid residue corresponding to one of amino acids 451 to 461 of AAV9 capsid protein (amino acid sequence SEQ ID NO:74 and see FIG. 7 for alignment of capsid protein amino acid sequence of other AAV serotypes with amino acid sequence of the AAV9 capsid and Table 17 for other capsid sequences), where said peptide insertion is surface exposed when the capsid protein is packaged as an AAV particle. The peptide insertion should not delete any residues of the AAV capsid protein. Generally, the peptide insertion occurs in a variable (poorly conserved) region of the capsid protein, compared with other serotypes, and in a surface exposed loop.

A peptide insertion described as inserted “at” a given site refers to insertion immediately after, that is having a peptide bond to the carboxy group of, the residue normally found at that site in the wild type virus. For example, insertion at Q588 in AAV9 means that the peptide insertion appears between Q588 and the consecutive amino acid (A589) in the AAV9 wildtype capsid protein sequence (SEQ ID NO:74). In embodiments, there is no deletion of amino acid residues at or near (within 5, 10, 15 residues or within the structural loop that is the site of the insertion) the point of insertion.

In particular embodiments, the capsid protein is an AAV9 capsid protein and the insertion occurs immediately after at least one of the amino acid residues 451 to 461. In particular embodiments, the peptide insertion occurs immediately after amino acid I451, N452, G453, S454, G455, Q456, N457, Q458, Q459, T460, or L461 of the AAV9 capsid (amino acid sequence SEQ ID NO:74). In certain embodiments, the peptide is inserted between residues S454 and G455 of AAV9 capsid protein or between the residues corresponding to S454 and G455 of an AAV capsid protein other than an AAV9 capsid protein (amino acid sequence SEQ ID NO: 74).

In other embodiments, provided are engineered capsid proteins comprising targeting peptides heterologous to the capsid protein that are inserted into the AAV capsid protein such that, when incorporated into the AAV vector the heterologous peptide is surface exposed.

In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), serotype rh74 (AAVrh74, versions 1 and 2), serotype rh34 (AAVrh34), serotype hu26 (AAVhu26), serotype rh31 (AAVrh31), serotype hu56 (AAVhu56), serotype hu53 (AAVhu53), serotype rh64R1 (AAVrh64R1), serotype rh46 (AAVrh46), and serotype rh73 (AAVrh73) (see FIG. 7 or Table 17), and the insertion occurs immediately after an amino acid residue corresponding to at least one of the amino acid residues 451 to 461. The alignments of these different AAV serotypes, as shown in FIG. 7, indicates “corresponding” amino acid residues in the different capsid amino acid sequences such that a “corresponding” amino acid residue is lined up at the same position in the alignment as the residue in the reference sequence. In some particular embodiments, the peptide insertion occurs immediately after one of the amino acid residues within: 450-459 of AAV1 capsid (SEQ ID NO:63); 449-458 of AAV2 capsid (SEQ ID NO:64); 449-459 of AAV3 capsid (SEQ ID NO:65); 443-453 of AAV4 capsid (SEQ ID NO:68); 442-445 of AAV5 capsid (SEQ ID NO:70); 450-459 of AAV6 capsid (SEQ ID NO:71); 451-461 of AAV7 capsid (SEQ ID NO: 72); 451-461 of AAV8 capsid (SEQ ID NO:73); 451-461 of AAV9 capsid (SEQ ID NO: 74); 452-461 of AAV9e capsid (SEQ ID NO:75); 452-461 of AAVrh10 capsid (SEQ ID NO: 76); 452-461 of AAVrh20 capsid (SEQ ID NO:77); 452-461 of AAVhu.37 (SEQ ID NO: 85); 452-461 of AAVrh74 (SEQ ID NO:80 or SEQ ID NO:81); or 452-461 of AAVrh39 (SEQ ID NO:78), in the sequences depicted in FIG. 7. In certain embodiments, the rAAV capsid protein comprises a peptide insertion immediately after (i.e., C-terminal to) amino acid 588 of AAV9 capsid protein (having the amino acid sequence of SEQ ID NO:74 and see FIG. 7), where said peptide insertion is surface exposed when the capsid protein is packaged as an AAV particle. In other embodiments, the rAAV capsid protein has a peptide insertion that is not immediately after amino acid 588 of AAV9 or corresponding to amino acid 588 of AAV9.

In specific embodiments, the peptide is inserted after 138; 262-272; 450-459; or 585-593 of AAV1 capsid (SEQ ID NO:63); 138; 262-272; 449-458; or 584-592 of AAV2 capsid (SEQ ID NO:64); 138; 262-272; 449-459; or 585-593 of AAV3 capsid (SEQ ID NO:65); 137; 256-262; 443-453; or 583-591 of AAV4 capsid (SEQ ID NO:68); 137; 252-262; 442-445; or 574-582 of AAV5 capsid (SEQ ID NO:70); 138; 262-272; 450-459; 585-593 of AAV6 capsid (SEQ ID NO:71); 138; 263-273; 451-461; 586-594 of AAV7 capsid (SEQ ID NO:72); 138; 263-274; 452-461; 587-595 of AAV8 capsid (SEQ ID NO:73); 138; 262-273; 452-461; 585-593 of AAV9 capsid (SEQ ID NO:74); 138; 262-273; 452-461; 585-593 of AAV9e capsid (SEQ ID NO:75); 138; 263-274; 452-461; 587-595 of AAVrh10 capsid (SEQ ID NO:76); 138; 263-274; 452-461; 587-595 of AAVrh20 capsid (SEQ ID NO:77); 138; 263-274; 452-461; 587-595 of AAVrh74 capsid (SEQ ID NO:80 or SEQ ID NO:81), 138; 263-274; 452-461; 587-595 of AAVhu37 capsid (SEQ ID NO:85); or 138; 263-274; 452-461; 587-595 of AAVrh39 capsid (SEQ ID NO:78) (as numbered in FIG. 7).

Generally, the peptide insertion is sequence of contiguous amino acids from a heterologous protein or domain thereof. The peptide to be inserted typically is long enough to retain a particular biological function, characteristic, or feature of the protein or domain from which it is derived. The peptide to be inserted typically is short enough to allow the capsid protein to form a coat, similarly or substantially similarly to the native capsid protein without the insertion. In preferred embodiments, the peptide insertion is from about 4 to about 30 amino acid residues in length, about 4 to about 20, about 4 to about 15, about 5 to about 10, or about 7 amino acids in length. The peptide sequences for insertion are at least 4 amino acids in length and may be 5, 6, 7, 8, 9, 10, 11, 12, 13, 14, or 15 amino acids in length. In some embodiments, the peptide sequences are 16, 17, 18, 19, or 20 amino acids in length. In embodiments, the peptide is no more than 7 amino acids, 10 amino acids or 12 amino acids in length.

A “peptide insertion from a heterologous protein” in an AAV capsid protein refers to an amino acid sequence that has been introduced into the capsid protein and that is not native to any AAV serotype capsid. Non-limiting examples include a peptide of a human protein in an AAV capsid protein.

In some embodiments, the rAAVs described herein increase tissue-specific (such as, but not limited to, CNS or skeletal and/or cardiac muscle) cell transduction in a subject (a human, non-human-primate, or mouse subject) or in cell culture, compared to the rAAV not comprising the amino acid substitution. In some embodiments, the increase in tissue specific cell transduction is at least 2, 10, 20, 30, 40, 50, 60, 70, 80, 90, or 100 fold more than that without the peptide insertion. For example, in some embodiments, there is a 50-80 fold increase in tissue specific cell transduction compared to transduction with the same AAV type without the modification. The increase in transduction may be assessed using methods described in the Examples herein and known in the art.

In some embodiments, the rAAVs described herein increase the incorporation of rAAV genomes into a cell or tissue type, particularly CNS or heart and/or skeletal muscle in a subject (a human, non-human primate or mouse subject) or in cell culture to the rAAV not comprising the peptide insertion. In some embodiments, the increase in genome integration is at least 2, 10, 20, 30, 40, 50, 60, 70, 80, 90, or 100 fold more than an AAV having a capsid without the peptide insertion. For example, in some embodiments, there is a 50-80 fold increase in genome integration compared to genome integration with the same AAV type without a peptide insert.

In another aspect, provided are libraries of capsids, including heterologous peptide insertion libraries or libraries of capsids having one or more amino acid substitutions. A heterologous peptide insertion library refers to a collection of rAAV vectors that carry the same peptide insertion at different insertion sites in the virus capsid, e.g., at different positions within a given variable region of the capsid or different variant peptides or even one or more amino acid substitutions. Provided are methods of screening the rAAVs having capsids from the library for enhance of improved properties such as tissue tropism, including enhanced transduction in CNS or cardiac and/or skeletal muscle tissue and, including, reduced transduction in liver and/or DRG cells. Generally, the capsid proteins used comprise AAV genomes that contain modified rep and cap sequences to prevent the replication of the virus under conditions in which it could normally replicate (co-infection of a mammalian cell along with a helper virus such as adenovirus). The members of the peptide insertion libraries may then be assayed for functional display of the peptide on the rAAV surface, tissue targeting and/or gene transduction.

### 5.2.3 Additional AAV Capsid Insertion Sites

The follow summarizes insertion sites for the peptides described herein immediately after amino acid residues of AAV capsids as set forth below (see also, FIG. 7):
AAV1: 138; 262-272; 450-459; 595-593; and in a particular embodiment, between 453-454 (SEQ ID NO:63).
AAV2: 138; 262-272; 449-458; 584-592; and in particular embodiment, between 452-453 (SEQ ID NO:64).
AAV3: 138; 262-272; 449-459; 585-593; and in particular embodiment, between 452-453 (SEQ ID NO:65).
AAV4: 137; 256-262; 443-453; 583-591; and in particular embodiment, between 446-447 (SEQ ID NO:68).
AAV5: 137; 252-262; 442-445; 574-582; and in particular embodiment, between 445-446 (SEQ ID NO:70).
AAV6: 138; 262-272; 450-459; 585-593; and in particular embodiment, between 452-453 (SEQ ID NO:71).
AAV7: 138; 263-273; 451-461; 586-594; and in particular embodiment, between 453-454 (SEQ ID NO:72).
AAV8: 138; 263-274; 451-461; 587-595; and in particular embodiment, between 453-454 (SEQ ID NO:73).
AAV9: 138; 262-273; 452-461; 585-593; and in particular embodiment, between 454-455 (SEQ ID NO:74).
AAV9e: 138; 262-273; 452-461; 585-593; and in particular embodiment, between 454-455 (SEQ ID NO:75).
AAVrh10: 138; 263-274; 452-461; 587-595; and in particular embodiment, between 454-455 (SEQ ID NO:76).
AAVrh20: 138; 263-274; 452-461; 587-595; and in particular embodiment, between 454-455 (SEQ ID NO:77).
AAVrh39: 138; 263-274; 452-461; 587-595; and in particular embodiment, between 454-455 (SEQ ID NO:78).
AAVrh74: 138; 263-274; 452-461; 587-595; and in particular embodiment, between 454-455 (SEQ ID NO:80 or SEQ ID NO:81).
AAVhu.37:138; 263-274; 452-461; 587-595; and in particular embodiment, between 454-455 (SEQ ID NO:85)

In particular embodiments, the peptide insertion occurs between amino acid residues 588-589 of the AAV9 capsid, or between corresponding residues of another AAV type capsid as determined by an amino acid sequence alignment (for example, as in FIG. 7). In particular embodiments, the peptide insertion occurs immediately after amino acid residue I451 to L461, S268 and Q588 of the AAV9 capsid sequence, or immediately after corresponding residues of another AAV capsid sequence (FIG. 7).

In some embodiments, one or more peptide insertions can be used in a single system. In some embodiments, the capsid is chosen and/or further modified to reduce recognition of the AAV particles by the subject's immune system, such as avoiding pre-existing antibodies in the subject. In some embodiments. In some embodiments, the capsid is chosen and/or further modified to enhance desired tropism/targeting.

### 5.2.4 AAV Vectors

Also provided are AAV vectors comprising the engineered capsids. In some embodiments, the AAV vectors are non-replicating and do not include the nucleotide sequences encoding the rep or cap proteins (these are supplied by the packaging cells in the manufacture of the rAAV vectors). In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAVrh34, AAVhu26, AAVrh31, AAVhu56, AAVhu53, AAVrh64R1, AAVrh46, and AAVrh73, or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, AAVrh34, AAVhu26, AAVrh31, AAVhu56, AAVhu53, AAVrh64R1, AAVrh46, and AAVrh73, or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV21YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, AAVrh34, AAVhu26, AAVrh31, AAVhu56, AAVhu53, AAVrh64R1, AAVrh46, and AAVrh73, or a derivative, modification, or pseudotype thereof. These engineered AAV vectors may comprise a genome comprising a transgene encoding a therapeutic protein or therapeutic nucleic acid, operably linked to regulatory sequences for expression in the target cells and flanked by AAV ITR sequences.

In particular embodiments, the recombinant AAV for use in compositions and methods herein is Anc80 or Anc80L65 (see, e.g., Zinn et al., 2015, Cell Rep. 12 (6): 1056-1068, which is incorporated by reference in its entirety). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLOS One 8 (4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23:857-862 and Georgiadis et al., 2018, Gene Therapy 25:450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29 (9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO:2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) may be used. In certain embodiments, a self-complementary vector, e.g., scAAV, may be used (see, e.g., Wu, 2007, Human Gene Therapy, 18 (2): 171-82; McCarty et al, 2001, Gene Therapy, 8 (16): 1248-1254; U.S. Pat. Nos. 6,596,535; 7,125,717; and 7,456,683, each of which is incorporated herein by reference in its entirety).

5.3. Methods of Making rAAV Particles

Another aspect of the present invention involves making rAAV particles having the capsids disclosed herein. In some embodiments, an rAAV particle is made by providing a nucleotide comprising the nucleic acid sequence encoding any of the capsid proteins described herein; and using a packaging cell system to prepare corresponding rAAV particles with capsid coats made up of the capsid protein. In some embodiments, the nucleic acid sequence encodes a sequence having at least 60%, 70%, 80%, 85%, 90%, 91%, 92%, 93%, 94%, 95%, 96%, 97%, 98%, 99% or 99.9%, identity to the sequence of a capsid protein molecule described herein, and retains (or substantially retains) biological function of the capsid protein. In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the one of the capsid proteins described herein, for example, those with sequences in Table 17 or otherwise described herein (see also FIG. 7), while retaining (or substantially retaining) biological function of the capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In other embodiments, the cap and rep genes are provided by a packaging cell and not present in the viral genome.

In some embodiments, the nucleic acid encoding the engineered capsid protein is cloned into an AAV Rep-Cap helper plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging. Non-limiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of

AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In some embodiments, the rAAVs provide transgene delivery vectors that can be used in therapeutic and prophylactic applications, as discussed in more detail below. In some embodiments, the rAAV vector also includes regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue. In specific embodiments, the AAV vector comprises a regulatory sequence, such as a promoter, operably linked to the transgene that allows for expression in target tissues. The promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, CAG promoter (SEQ ID NO:101), RPE65 promoter, opsin promoter, the TBG (Thyroxine-binding Globulin) promoter, the APOA2 promoter, SERPINA1 (hAAT) promoter, or MIR122 promoter. In some embodiments, particularly where it may be desirable to turn off transgene expression, an inducible promoter is used, e.g., hypoxia-inducible or rapamycin-inducible promoter.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the a capsid protein described herein (see Table 17, e.g.), while retaining the biological function of the engineered capsid. In certain embodiments, the encoded engineered capsid has the sequence of an AAV8.BBB.LD capsid (SEQ ID NO:27), an AAV9.BBB.LD capsid (SEQ ID NO: 29), an AAV9.496-NNN/AAA-498 capsid (SEQ ID NO:31), AAV9.496-NNN/AAA-498. W503R capsid (SEQ ID NO:32), AAV9.W503R capsid (SEQ ID NO:33), AAV9.Q474A capsid (SEQ ID NO:34), AAV9.N272A.496-NNN/AAA-498 capsid (SEQ ID NO:49) or AAV9.N266A.496-NNN/AAA-498 capsid (SEQ ID NO:50). Also provided are engineered AAV vectors other than AAV9 vectors, such as engineered AAV1, AAV2, AAV3, AAV4, AAV5, AAV6, AAV7, AAV8, AAV9, AAV9e, AAVrh10, AAVrh20, AAVhu.37, AAVrh39, AAVrh74, AAVrh34, AAVhu26, AAVrh31, AAVhu56, AAVhu53, AAVrh.46, AAVrh.64.R1, AAV.rh.73 vectors, including with the amino acid substitutions and/or peptide insert as described herein and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to the wild type or unengineered sequence for that AAV type and that retains its biological function.

The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12: S18-S27, which is incorporated by

In certain embodiments, nucleic acids sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) control elements, which include a) a promoter and, optionally, enhancer elements to promote expression of the transgene in CNS and/or muscle cells, b) optionally an intron sequence, such as a chicken β-actin intron, and c) a polyadenylation sequence, such as an SV40 poly A or rabbit β-globin poly A signal; and (3) transgene providing (e.g., coding for) a nucleic acid or protein product of interest.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl2 sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or other cell types may be used, for example, but not limited, to HuH-7, HEK293, fibrosarcoma HT-1080, HKB-11, and CAP cells. Once expressed, characteristics of the expressed product (i.e., transgene product) can be determined, including determination of the glycosylation and tyrosine sulfation patterns, using assays known in the art.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 1A-1B below provides a list of transgenes that may be used in any of the rAAV vectors described herein, in particular, in the novel insertion sites described herein, to treat or prevent the disease with which the transgene is associated, also listed in Tables 1A-1B. As described herein, the AAV vector may be engineered as described herein to target the appropriate tissue for delivery of the transgene to effect the therapeutic or prophylactic use. The appropriate AAV serotype may be chosen to engineer to optimize the tissue tropism and transduction of the vector.

TABLE 1A

| | | Possible |
|---|---|---|
| | | AAV |
| | | serotype for |
| | | delivery of |
| Disease | Transgene | transgene |
| MPS I | alpha-L-iduronidase (IDUA) | AAV9 |
| MPS II (Hunter | iduronate-2-sulfatase (IDS) | AAV9 |
| Syndrome) | | |
| ceroid lipofuscinosis | (CLN1, CLN2, CLN10, CLN13), a soluble | AAV9 |
| (Batten disease) | lysosomal protein (CLN5), a protein in the | |
| | secretory pathway (CLN11), two cytoplasmic | |
| | proteins that also peripherally associate with | |
| | membranes (CLN4, CLN14), and many | |
| | transmembrane proteins with different subcellular | |
| | locations (CLN3, CLN6, CLN7, CLN8, CLN12) | |
| MPS IIIa (Sanfilippo | heparan sulfate sulfatase (also called N- | AAV9, Rh10 |
| type A Syndrome) | sulfoglucosamine sulfohydrolase (SGSH)) | |
| MPS IIIB (Sanfilippo | N-acetyl-alpha-D-glucosaminidase (NAGLU) | AAV9 |
| type B Syndrome) | | |
| MPS VI (Maroteaux- | arylsulfatase B | AAV8 |
| Lamy Syndrome) | | |
| Morquio syndrome | Beta galactosidase or galactosamine-6-sulfatase | AAV9 |
| (MPS IV) | | |
| Gaucher disease (type | Glucocerebrosidase, GBA1 | AAV9 |
| I, II and III) | | |
| Parkinson's Disease | Glucocerebrosidase; GBA1 | AAV9 |
| (or levodopa-induced | | |
| dyskinesia in | | |
| Parknison's disease) | | |
| Parkinson's Disease | dopamine decarboxylase | AAV2 |
| (or levodopa-induced | | |
| dyskinesia in | | |
| Parknison's disease) | | |
| Pompe | acid maltase; GAA | AAV9 |
| Metachromatic | Aryl sulfatase A | Rh10 |
| leukodystrophy | | |
| MPS VII (Sly | beta-glucuronidase | |
| syndrome) | | |
| MPS VIII | glucosamine-6-sulfate sulfatase | |
| MPS IX | hyaluronidase | |
| Niemann-Pick disease | sphingomyelinase | |
| Niemann-Pick disease | a npc1 gene encoding a | |
| without | cholesterol metabolizing enzyme | |
| sphingomyelinase | | |
| deficiency | | |
| Tay-Sachs disease | Alpha subunit of beta-hexosaminidase | |
| Sandhoff disease | both alpha and beta subunit of beta- | |
| | hexosaminidase | |
| Fabry Disease | alpha-galactosidase | |
| Fucosidosis | Fucosidase (FUCA1 gene) | |
| Alpha-mannosidosis | alpha-mannosidase | |
| Beta-mannosidosis | Beta-mannosidase | |
| Wolman disease | cholesterol ester hydrolase | |
| Parkinson's disease | Neurturin | |
| (or levodopa-induced | | |
| dyskinesia in | | |
| Parknison's disease) | | |
| Parkinson's disease | glial derived growth factor (GDGF) | |
| (or levodopa-induced | | |
| dyskinesia in | | |
| Parknison's disease) | | |
| Parkinson's disease | tyrosine hydroxylase | |
| (or levodopa-induced | | |
| dyskinesia in | | |
| Parknison's disease) | | |
| Parkinson's disease | glutamic acid decarboxylase. | |
| (or levodopa-induced | | |
| dyskinesia in | | |
| Parknison's disease) | | |
| No disease listed | fibroblast growth factor-2 (FGF-2) | |
| No disease listed | brain derived growth factor (BDGF) | |
| No disease listed | neuraminidase deficiency with betagalactosidase | |
| (Galactosialidosis | deficiency | |
| (Goldberg syndrome)) | | |
| Spinal Muscular | SMN | AAV9 |
| Atrophy (SMA) | | |
| Friedreich's ataxia | Frataxin | AAV9 |
| | | PHP.B |
| Amyotrophic lateral | SOD1 | Rh10 |
| sclerosis (ALS) | | |
| Glycogen Storage | Glucose-6-phosphatase | AAV8 |
| Disease 1a | | |
| XLMTM | MTM1 | AAV8 or |
| | | AAV9 |
| Crigler Najjar | UGT1A1 | AAV8 |
| CPVT | CASQ2 | AAV9 |
| Rett syndrome | MECP2 | AAV9 |
| Achromatopsia | CNGB3, CNGA3, GNAT2, PDE6C | AAV8 |
| Choroidermia | CDM | AAV8 |
| Danon Disease | LAMP2 | AAV9 |

TABLE 1B

| | | Possible AAV |
|---|---|---|
| | | serotype for |
| | | delivery of |
| Disease | Transgene | transgene |
| Cystic Fibrosis | CFTR | AAV2 |
| Duchenne Muscular Dystrophy | Mini-Dystrophin Gene or | AAV2, AAV8 |
| | micro-dystrophin gene | or AAV9 |
| Limb Girdle Muscular Dystrophy Type | human-alpha-sarcoglycan | AAV1 |
| 2C\|Gamma-sarcoglycanopathy | | |
| Advanced Heart Failure | SERCA2a | AAV6 |
| Rheumatoid Arthritis | TNFR:Fc Fusion Gene | AAV2 |
| Leber Congenital Amaurosis | GAA | AAV1 |
| Limb Girdle Muscular Dystrophy Type | gamma-sarcoglycan | AAV1 |
| 2C\|Gamma-sarcoglycanopathy | | |
| Retinitis Pigmentosa | hMERTK | AAV2 |
| Age-Related Macular Degeneration | sFLT01 | AAV2 |
| Becker Muscular Dystrophy and | huFollistatin344 | AAV1 |
| Sporadic Inclusion Body Myositis | | |
| Parkinson's Disease (or levodopa- | GDNF | AAV2 |
| induced dyskinesia in Parknison's | | |
| disease) | | |
| Metachromatic Leukodystrophy (MLD) | cuARSA | AAVrh.10 |
| Hepatitis C | anti-HCV shRNA | AAV8 |
| Limb Girdle Muscular Dystrophy Type | hSGCA | AAVrh74* |
| 2D | | |
| Human Immunodeficiency Virus | PG9DP | AAV1 |
| Infections; HIV Infections (HIV-1) | | |
| Acute Intermittant Porphyria | PBGD | AAV5 |
| Leber's Hereditary Optical Neuropathy | P1ND4v2 | AAV2 |
| Alpha-1 Antitrypsin Deficiency | alpha1AT | AAVrh10 |
| Pompe Disease | hGAA | AAV9 |
| X-linked Retinoschisis | RS1 | AAV8 |
| Choroideremia | hCHM | AAV2 |
| Giant Axonal Neuropathy | JeT-GAN | AAV9 |
| Duchenne Muscular Dystrophy | micro-Dystrophin | AAV8; |
| | | AAVrh74* |
| X-linked Retinoschisis | hRS1 | AAV2 |
| Squamous Cell Head and Neck Cancer; | hAQP1 | AAV2 |
| Radiation Induced Xerostomia | | |
| Hemophilia B | Factor IX | AAVrh10/ |
| | | Rh74 |
| Homozygous FH | hLDLR | AAV8 |
| Dysferlinopathies | rAAVrh74.MHCK7.DYSF.DV | AAVrh74 |
| Hemophilia B | AAV6 ZFP nuclease | AAV6 |
| MPS I | AAV6 ZFP nuclease | AAV6 |
| Rheumatoid Arthritis | NF-kB.IFN-β | AAV5 |
| Batten/CLN6 | CLN6 | AAV9 |
| Sanfilippo Disease Type A | hSGSH | AAV9 |
| Osteoarthritis | 5IL-1Ra | AAV2.5 |
| Achromatopsia | CNGA3 | AAV2tYF |
| Achromatopsia | CNGB3 | AAV8 |
| Ornithine Transcarbamylase (OTC) | OTC | scAAV8 |
| Deficiency | | |
| Hemophilia A | Factor VIII | LK03/AAV3B |
| Mucopolysaccharidosis II | ZFP nuclease | AAV6 |
| Hemophilia A | ZFP nuclease | AAV6 |
| Wet AMD | anti-VEGF | AAV8 |
| X-Linked Retinitis Pigmentosa | PGR | AAV2 |
| Mucopolysaccharidosis Type VI | hARSB | AAV8 |
| Leber Hereditary Optic Neuropathy | ND4 | AAV2 |
| X-Linked Myotubular Myopathy | MTM1 | AAV8 |
| Crigler-Najjar Syndrome | UGT1A1 | AAV8 |
| Achromatopsia | CNGB3 | AAV8 |
| Retinitis Pigmentosa | hPDE6B | AAV5 |
| X-Linked Retinitis Pigmentosa | RPGR | AAV2tYF |
| Mucopolysaccharidosis Type 3 B | hNAGLU | AAV9 |
| Duchenne Muscular Dystrophy | GALGT2 | AAV8; |
| | | AAVrh74 |
| Arthritis, Rheumatoid; Arthritis, | TNFR:Fc Fusion Gene | AAV2 |
| Psoriatic; Ankylosing Spondylitis | | |
| Idiopathic Parkinson's Disease (or | Neurturin | AAV2 |
| levodopa-induced dyskinesia in | | |
| Parknison's disease) | | |
| Alzheimer's Disease | NGF | AAV2 |
| Human Immunodeficiency Virus | tgAAC09 | AAV2 |
| Infections; HIV Infections (HIV-1) | | |
| Familial Lipoprotein Lipase Deficiency | LPL | AAV1 |
| Idiopathic Parkinson's Disease (or | Neurturin | AAV2 |
| levodopa-induced dyskinesia in | | |
| Parknison's disease) | | |
| Alpha-1 Antitrypsin Deficiency | hAAT | AAV1 |
| Leber Congenital Amaurosis (LCA) 2 | hRPE65v2 | AAV2 |
| Batten Disease; Late Infantile | CLN2 | AAVrh.10 |
| Neuronal Lipofuscinosis | | |
| Parkinson's Disease (or levodopa- | GAD | AAV2 |
| induced dyskinesia in Parknison's | | |
| disease) | | |
| Sanfilippo Disease Type A/ | N-sulfoglucosamine | AAVrh.10 |
| Mucopolysaccharidosis Type IIIA | sulfohydrolase (SGSH) gene | |
| Congestive Heart Failure | SERC2a | AAV1 |
| Becker Muscular Dystrophy and | rAAV1.CMV.huFollistatin344 | AAV1 |
| Sporadic Inclusion Body Myositis | | |
| Parkinson's Disease (or levodopa- | hAADC-2 | AAV2 |
| induced dyskinesia in Parknison's | | |
| disease) | | |
| Choroideremia | REP1 | AAV2 |
| CEA Specific AAV-DC-CTL | CEA | AAV2 |
| Treatment in Stage IV Gastric Cancer | | |
| Gastric Cancer | MUC1-peptide-DC-CTL | |
| Leber's Hereditary Optical Neuropathy | scAAV2-P1ND4v2 | scAAV2 |
| Aromatic Amino Acid Decarboxylase | hAADC | AAV2 |
| Deficiency | | |
| Hemophilia B | Factor IX | AAVrh10 |
| Parkinson's Disease (or levodopa- | AADC | AAV2 |
| induced dyskinesia in Parknison's | | |
| disease) | | |
| Leber Hereditary Optic Neuropathy | Genetic:GS010\|Drug:Placebo | AAV2 |
| SMA—Spinal Muscular Atrophy\|Gene | SMN | AAV9 |
| Therapy | | |
| Hemophilia A | B-Domain Deleted Factor VIII | AAV8 |
| MPS I | IDUA | AAV9 |
| MPS II | IDS | AAV9 |
| CLN3-Related Neuronal Ceroid- | CLN3 | AAV9 |
| Lipofuscinosis (Batten) | | |
| Limb-Girdle Muscular Dystrophy, Type | hSGCB | rh74 |
| 2E | | |
| Alzheimer Disease | APOE2 | rh10 |
| Retinitis Pigmentosa | hMERKTK | AAV2 |
| Retinitis Pigmentosa | RLBP1 | AAV8 |
| Wet AMD | Anti-VEGF antibody | AAV2.7m8 |

For example, a rAAV vector comprising a transgene encoding glial derived growth factor (GDGF) finds use treating/preventing/managing Parkinson's disease and/or levodopa-induced dyskinesia (LID) in Parkinson's disease (PD-LID). Generally, the rAAV vector is administered systemically but may also be administered directly to the CNS, for example to the striatum by intraparenchymal administration or otherwise. For example, the rAAV vector may also be provided by intravenous, intrathecal, intra-nasal, and/or intra-peritoneal administration. In embodiments, the rAAV for delivery of the therapeutic for Parkinson's disease and/or levodopa-induced dyskinesia (LID) in Parkinson's disease (PD-LID) is a capsid having an insert of the TFR3 peptide (RTIGPSV, SEQ ID NO: 19), including in the VR-IV loop of the capsid (see FIGS. 2 and 7) and, in embodiments, is inserted between S454 and G455 of the AAV9 capsid, or corresponding position in another appropriate capsid protein (see FIG. 7 for alignment), and may be AAV9S454-TFR3 (SEQ ID NO:42). As shown in Example 9, the AAV9S454-TFR3 capsid has enhanced abundance in the putamen ipsilateral and caudate relative to the other capsids in the library when injected in the striatum/putamen of an NHP and is further localized to regions of the brain such as the substantia nigra, intralaminar thalamus (rostral and caudal), frontal cortex, putamen, caudate and globus pallidus. As also shown in Example 9, the AAV9S454-TFR3 capsid has superior transduction and enhanced transcriptional activity (3 to 4 fold increase) putamen, caudate, substantia nigra, intralaminar thalamus and frontal cortex after intraparenchymal administration in NHPs, relative to the parental AAV9 capsid. Similar results are seen in mice upon ICV administration, with favorable reduction in biodistribution in liver, heart and DRG in both mice and NHP following ICV administration. Based upon the pattern of localization, the capsids may be transported by retrograde and/or anterograde transport from the site of injection to the substantia nigra. Such capsids may be useful for delivering transgenes encoding a therapeutic protein or therapeutic nucleic acid for diseases associated with dopaminergic neurons, such as Parkinson's disease and/or levodopa-induced dyskinesia (LID) in Parkinson's disease (PD-LID) and other movement disorders, such as tardive dyskinesia, or, alternatively, schizophrenia.

Accordingly, provided are methods of treating such CNS disorders associated with dopaminergic neurons by administration, including intraparenchymal administration to the CNS, including the striatum or putamen, of an rAAV having a capsid comprising a TFR3 peptide, including inserted in the VR-IV loop, including between positions S454 and G455 of AAV9 (or other capsid protein, for example based upon the alignment in FIG. 7), and may be AAV9S454-TFR3 capsid (SEQ ID NO:42) or a variant thereof, wherein the rAAV comprises an expression cassette with a transgene encoding a protein therapeutic for the CNS disorder associated with dopaminergic neurons operably linked to regulatory elements for expression in the CNS and, including, in dopaminergic neurons.

In particular aspects, the rAAVs of the present invention find use in delivery to target tissues, or target cell types, including cell matrix associated with the target cell types, associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject tan rAAV where the peptide insertion is a homing peptide. In the case of Parkinson's disease and/or levodopa-induced dyskinesia (LID) in Parkinson's disease (PD-LID), for example, a rAAV vector comprising a peptide insertion that directs the rAAV to neural tissue can be used, in particular, where the peptide insertion facilitates the rAAV in crossing the blood brain barrier to the CNS or facilitates the rAAV in retrograde or anterograde transport in the CNS.

For a disease or disorder associated with neural tissue, an rAAV vector can be used that comprises a peptide insertion from a neural tissue-homing domain, such as any described herein. Diseases/disorders associated with neural tissue include Alzheimer's disease, amyotrophic lateral sclerosis (ALS), amyotrophic lateral sclerosis (ALS), Battens disease, Batten's Juvenile NCL form, Canavan disease, chronic pain, Friedreich's ataxia, glioblastoma multiforme, Huntington's disease, Late Infantile neuronal ceroid lipofuscinosis (LINCL), lysosomal storage disorders, Leber's congenital amaurosis, multiple sclerosis, Parkinson's disease and/or levodopa-induced dyskinesia (LID) in Parkinson's disease (PD-LID), Pompe disease, Rett syndrome, spinal cord injury, spinal muscular atrophy (SMA), stroke, and traumatic brain injury. The vector further can contain a transgene for therapeutic/prophylactic benefit to a subject suffering from, or at risk of developing, the disease or disorder (see Tables 1A-1B).

The rAAV vectors of the invention also can facilitate delivery, in particular, targeted delivery, of oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. Also, the rAAV molecule of the invention may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Provided also are methods of determining a dosage for a human subject in need of treatment for a neurological disease or disorder for intraparenchymal administration of an AAV9 (or modified AAV9) gene therapy product, by comparing the therapeutically effective dosage in a mouse, rat or NHP model for the disease or disorder that achieves durable expression of the transgene encoding the therapeutic protein or therapeutic nucleic acid for at least 6 months. The dosage may be determined based upon the relative size of the brain of the human subject relative to the size of the brain of the animal model subject (for example, a log difference between rat and human or 2 log different between mice and human).

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once, or may be administered in a series of 2, 3 or 4 or more times, for example, weekly, monthly or every two months, 3 months, 6 months or one year until the series of doses has been administered.

The rAAV molecules of the invention may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In particular embodiments, such as where the transgene is intended to be expressed in the CNS, the vector is administered via lumbar puncture or via cisterna magna.

In certain embodiments, the agents of the invention are administered intravenously and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, rAAVs can be used for in vivo delivery of transgenes for scientific studies such as optogenetics, gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In certain embodiments, the agent of the invention is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

The following examples report an analysis of surface-exposed loops on the AAV9 capsid to identify candidates for capsid engineering via insertional mutagenesis. The invention is illustrated by way of examples, describing the construction of rAAV9 capsids engineered to contain 7-mer peptides designed on the basis of the human axonemal dynein heavy chain tail. Briefly, three criteria were used for selecting surface loops that might be amenable to short peptide insertions: 1) minimal side chain interactions with adjacent loops; 2) variable sequence and structure between serotypes (lack of conserved sequences); and 3) the potential for interrupting commonly targeted neutralizing antibody epitopes. A panel of peptide insertion mutants was constructed and the individual mutants were screened for viable capsid assembly, peptide surface exposure, and potency. The top candidates were then used as templates for insertion of homing peptides to test if these peptide insertion points could be used to re-target rAAV vectors to tissues of interest. Further examples, demonstrate the increased transduction and tissue tropism for certain of the modified AAV capsids described herein.

### 6.1. Example 1—Analysis of AAV9 Capsid

FIGS. 1 and 2 depict analysis of variable region four of the adeno-associated virus type 9 (AAV9 VR-IV) by amino acid sequence comparison to other AAVs VR-IV (FIG. 1) and protein model (FIG. 2). As seen, AAV9 VR-IV is exposed on the surface at the tip or outer surface of the 3-fold spike. Further analysis indicated that there are few side chain interactions between VR-IV and VR—V and that the sequence and structure of VR-IV is variable amongst AAV serotypes, and further that there is potential for interrupting a commonly-targeted neutralizing antibody epitope and thus, reducing immunogenicity of the modified capsid.

### 6.2. Example 2—Construction of AAV9 Mutants

Eight AAV9 mutants were constructed, to each include a heterologous peptide but at different insertion points in the VR-IV loop. The heterologous peptide was a FLAG tag that was inserted immediately following the following residues in vectors identified as pRGNX1090-1097, as shown in Table 2.

TABLE 2

| Vector designation | AAV9 VR-IV Insertion site for FLAG tag |
|---|---|
| pRGNX1090 | I451 |
| pRGNX1091 | N452 |
| pRGNX1092 | G453 |
| pRGNX1093 | S454 |
| pRGNX1094 | G455 |
| pRGNX1095 | Q456 |
| pRGNX1096 | N457 |
| pRGNX1097 | Q458 |

### 6.3. Example 3—Analysis of Packaging Efficiency

FIG. 3 depicts high packaging efficiency in terms of genome copies per mL (GC/mL) of wild type AAV9 and eight (8) candidate rAAV9 vectors (1090, 1091, 1092, 1093, 1094, 1095, 1096, and 1097), where the candidate vectors each contain a FLAG insert at different sites within AAV9's VR-IV. All vectors were packaged with luciferase transgene in 10 mL culture to facilitate determining which insertion points did not interrupt capsid packaging; error bars represent standard error of the mean.

As seen, all candidates package with high efficiency.

### 6.4. Example 4—Analysis of Surface FLAG Exposure

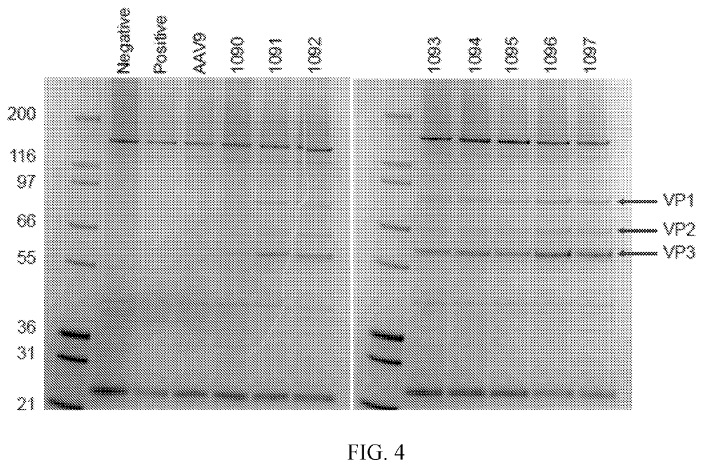

FIG. 4 depicts surface exposure of FLAG inserts in each of eight (8) candidate rAAV9 vectors (1090, 1091, 1092, 1093, 1094, 1095, 1096, and 1097), confirmed by immunoprecipitation of transduced vectors by binding to anti-FLAG resin. Binding to anti-FLAG indicates insertion points that allow formation of capsids that display the peptide insertion on the surface.

Transduced cells were lysed and centrifuged. 500 μL of cell culture supernatant was loaded on 20 uL agarose-FLAG beads and eluted with SDS-PAGE loading buffer also loaded directly on the gel. For a negative control, 293-ssc supernatant was used that contained no FLAG inserts.

As seen, 1090 had the lowest titer of the candidate vectors, indicating the least protein pulled down. Very low titers also were seen with the positive control. It is likely that not a sufficient amount of positive control had been loaded for visualization on SDS-PAGE.

### 6.5. Example 5—Analysis of Transduction Efficiency

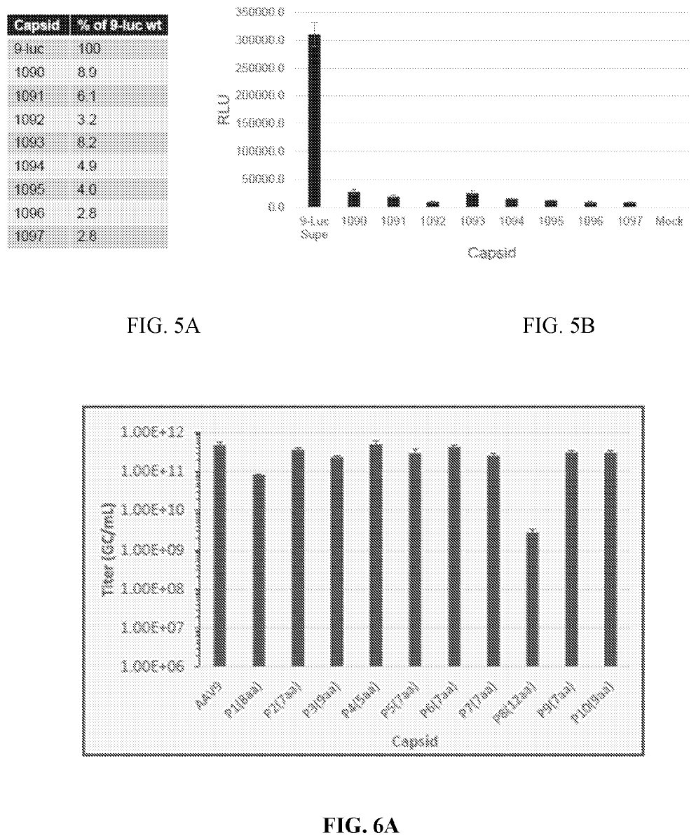

FIGS. 5A-5B depict transduction efficiency in Lec2 cells, transduced with capsid vectors carrying the luciferase gene as a transgene, that was packaged into either wild type AAV9 (9-luc), or into each of eight (8) candidate rAAV9 vectors (1090, 1091, 1092, 1093, 1094, 1095, 1096, and 1097); activity is expressed as percent luciferase activity, taking the activity of 9-luc as 100% (FIG. 5A), or as Relative Light Units (RLU) per microgram of protein (FIG. 5B).

CHO-derived Lec2 cells were grown in aMEM and 10% FBS. The Lec2 cells were transduced at a multiplicity of infection (MOI) of about 2×10⁸ GC vector (a MOI of about 10,000) and were treated with ViraDuctin reagent (similar results were observed on transducing Lec2 cells at a MOI of about 10,000 GC/cell but treated with 40 μg/mL zinc chloride (ZnCl2); results not shown). Lec2 cells are proline auxotrophs from CHO.

As seen, transduction efficiency in vitro is lower than that obtained using wild type AAV9 (9-luc). Nonetheless, previous studies have shown that introduction of a homing peptide can decrease in vitro gene transfer in non-target cells (such as 293, Lec2, or HeLa), while significantly increasing in vitro gene transfer in target cells (see, e.g., Nicklin et al. 2001; and Grifman et al. 2001).

### 6.6. Example 6—Analysis of Packaging Efficiency as a Factor of Insertion Peptide Composition and Length

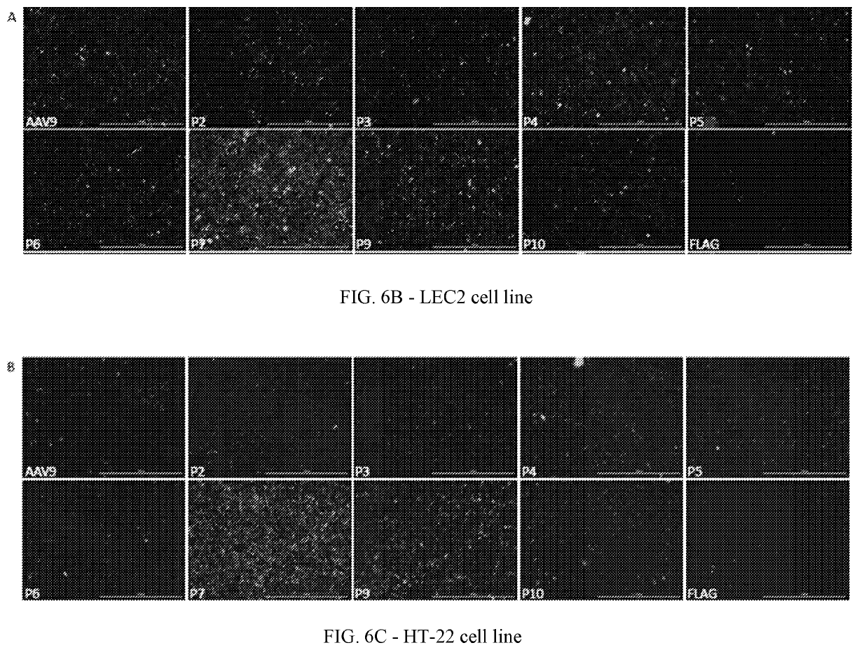

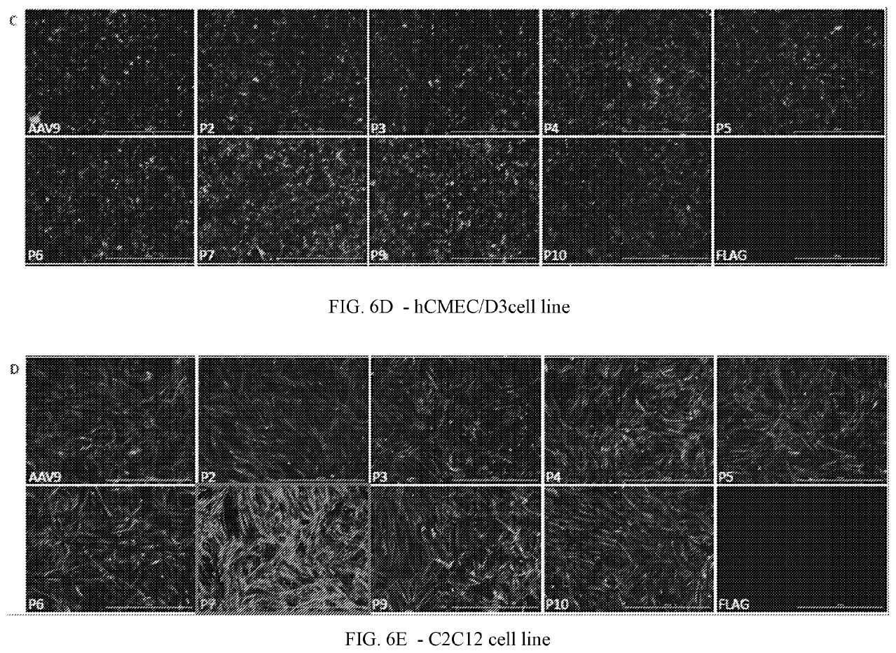

FIG. 6A depicts a bar graph illustrating that insertions immediately after S454 of AAV9 capsid (SEQ ID NO:74) of varying peptide length and composition may affect production efficiencies of AAV particles in a packaging cell line. Ten peptides of varying composition and length were inserted after S454 (between residues 454 and 455) within AAV9 VR-IV. qPCR was performed on harvested supernatant of transfected suspension HEK293 cells five days post-transfection. The results depicted in the bar graph demonstrate that the nature and length of the insertions may affect the ability of AAV particles to be produced at high titer and packaged in 293 cells. (Error bars represent standard error of the mean length of peptide, which is noted on the Y-axis in parenthesis.)

AAV9 vectors having a capsid protein containing a homing peptide of the following peptide sequences (Table 3) at the S454 insertion site were studied. Suspension-adapted HEK293 cells were seeded at 1×10⁶ cells/mL one day before transduction in 10 mL of media. Triple plasmid DNA transfections were done with PEIpro® (Polypus transfection) at a DNA: PEI ratio of 1:1.75. Cells were spun down and supernatant harvested five days post-transfection and stored at −80° C.

TABLE 3

| Peptide | Tissue or | | |
|---|---|---|---|
| | Target | Peptide | SEQ |
| # | Designation | Sequence | ID NO: |
| P1 | Bone1 (D8) | DDDDDDDD | 2 |
| P2 | Brain1 | LSSRLDA | 3 |
| P3 | Brain2 | CLSSRLDAC | 4 |
| P4 | Kidney1 | LPVAS | 6 |
| P5 | Kidney2 | CLPVASC | 5 |
| P6 | Muscle1 | ASSLNIA | 7 |
| P7 | TfR1 | HAIYPRH | 10 |
| P8 | TfR2 | THRPPMWSPVWP | 11 |
| P9 | TfR3 | RTIGPSV | 12 |
| P10 | TfR4 | CRTIGPSVC | 13 |

qPCR was performed on harvested supernatant of transfected suspension HEK293 cells five days post-transfection. Samples were subjected to DNase I treatment to remove residual plasmid or cellular DNA and then heat treated to inactivate DNase I and denature capsids. Samples were titered via qPCR using TaqMan Universal PCR Master Mix, No AmpEraseUNG (ThermoFisherScientific) and primer/probe against the polyA sequence packaged in the transgene construct. Standard curves were established using RGX-501 vector BDS.

Peptide insertions directly after S454 ranging from 5 to 10 amino acids in length produced AAV particles having adequate titer, whereas an upper size limit is possible, with significant packaging deficiencies observed for the peptide insertion having a length of 12 amino acids.

### 6.7. Example 7—Homing Peptides Alter the Transduction Properties of AAV9 In Vitro when Inserted after S454

FIGS. 6B-E depict fluorescence images of cell cultures of (FIG. 6B) Lec2 cell line (sialic acid-deficient epithelial cell line) (FIG. 6C) HT-22 cell line (neuronal cell line), (FIG. 6D) hCMEC/D3 cell line (brain endothelial cell line), and (FIG. 6E) C2C12 cell line (muscle cell line). AAV9 wild type and S454 insertion homing peptide capsids of Table 3 containing GFP transgene were used to transduce the noted cell lines.

Cell lines were plated at 5-20×10³ cells/well (depending on the cell line) in 96-well 24 hours before transduction. Cells were transduced with AAV9-GFP vectors (with or without insertions) at 1×10¹⁰ particles/well and analyzed via Cytation5 (BioTek) 48-96 hours after transduction, depending on the difference in expression rate in each cell line. Lec2 cells were cultured as in Example 5, blood-brain barrier hCMEC/D3 (EMD Millipore) cells were cultured according to manufacturer's protocol, HT-22 and HUH7 cells were cultured in DMEM and 10% FBS, and C2C12 myoblasts were plated in DMEM and 10% FBS and differentiated for three days pre-transfection in DMEM supplemented with 2% horse serum and 0.1% insulin. AAV9.S454.FLAG showed low transduction levels in every cell type tested.

Images show that homing peptides can alter the transduction properties of AAV9 in vitro when inserted after S454 in the AAV9 capsid protein, as compared to unmodified AAV9 capsid. P7 (TfR1 peptide, HAIYPRH (SEQ ID NO:10)) for all cell lines show the highest rate of transduction followed by P9 (TfR3 peptide, RTIGPSV (SEQ ID NO:12)). P4 (Kidney 1 peptide, LPVAS (SEQ ID NO:6)) showed a slightly higher rate of transduction than that of AAV9 wildtype for all cell types. Higher transduction rates were observed for P6 (Musclel peptide, ASSLNIA (SEQ ID NO:7)) in the brain endothelial hCMEC/D3 cell line and the C2C12 muscle cell line cultures as compared to the Lec2 and HT-22 cell line cultures. P1 vector was not included in images due to extremely low transduction efficiency, and P8 vector was not included due to low titer.

### 6.8. Example 8—Analysis of AAV Capsids for Peptide Insertion Points

FIG. 7 depicts alignment of AAVs 1-9e, rh10, rh20, rh39, rh74, hul2, hu21, hu26, hu37, hu51 and hu53 capsid sequences within insertion sites for capsid sequences within insertion sites for human peptides within or near the initiation codon of VP2, variable region 1 (VR-I), variable region 4 (VR-IV), and variable region 8 (VR-VIII) highlighted in grey; a particular insertion site within variable region eight (VR-VIII) of each capsid protein is shown by the symbol “#” (after amino acid residue 588 according to the amino acid numbering of AAV9).

### 6.19. Example 9 Study of CNS Biodistribution Following Intraparenchymal (IP) Administration of Capsid Library

Pooled barcoded vectors were administered to NHPs by intraparenchymal (IP or IPa) injection into the posterior region of the striatum specifically targeting the putamen. The pooled mixture consists of 118 different AAV capsids, including natural isolates and engineered AAVs, as described herein, expressing the GFP reporter gene from the universal CAG promoter (SEQ ID NO:101). The targeted CNS tissue was the putamen in this study, and capsids were identified that have distinct expression patterns locally and that demonstrate retrograde or anterograde transport.

### Study Design

Adult female cynomolgus monkeys (Macaca fascicularis) (n=3), ˜2-3 years of age with a weight of 2-3 kg were screened for neutralizing antibodies against naturally occurring AAV vectors (AAV2, AAV8, AAV9). A unilateral intraparenchymal injection into the putamen (left hemisphere) was performed with 100 uL at a flow rate of 1-2 uL per minute. Animals were perfused with PBS, sacrificed and tissues harvested 3 weeks post-injection. Harvested tissues were flash frozen until analysis by qPCR and NGS.

### Study Design

Study Design

| | | Number | | Dose | Dose | | |
|---|---|---|---|---|---|---|---|
| | | of | Infusion | Conc | Volume | Dose | Necropsy |
| Group | Test Article | Animals | Site | (gc/mL) | (μL) | (gc) | Day |
| 1 | NAV + Library | 3 Female | Putamen | 2.4e12 | 100 | 2.4e11 | 23 |

Conc = Concentration

Peripheral tissues analyzed were from liver, spleen, and heart. Cerebrospinal fluid (CSF) and brain sections were extracted, e.g. putamen (bilateral)-three tissue samples at different rostro-caudal levels; Caudate (bilateral); Cortex (mPFC, dlPFC, Cg, Frontal-M1, Parietal-S1, Occipital, Insular, Temporal (bilateral)); White matter of extreme capsule (bilateral); Globus pallidus (internal/external) (external-GPe); Basolateral amygdala (bilateral); Intralaminar thalamus (CM/Pf) (rostral and caudal); Thalamus (pulvinar); STN; Substantia nigra (SNr/SNc) (bilateral); Pedunculopontine tegmentum (PPT); Hippocampus; Cerebellum; Spinal Cord and DRG (cervical, lumber, thoracic).

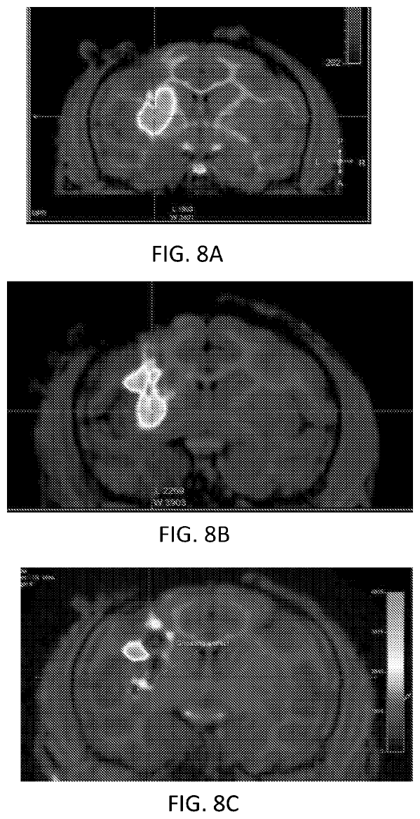

Dosing MRIs were obtained as T1 images taken following injection in each of the monkeys to visualize the location of the injection (Prohance contrast agent was added to injected test article) (FIG. 8).

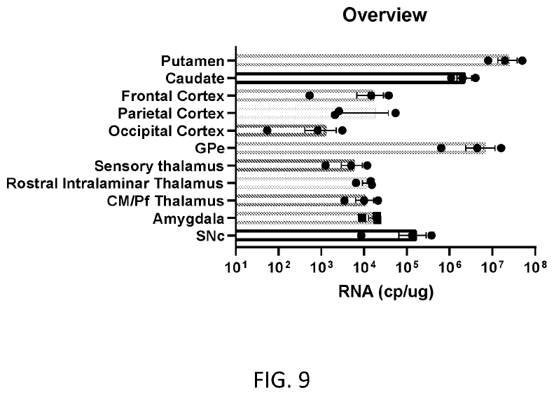

FIG. 9 depicts qPCR analysis which revealed high copy number (RNA cp/ug) of total vector transcripts localized to putamen, caudate, GPe and interestingly, SNc, as well as other tissues (RNA copy number/ug, adjusted to log scale). This distribution is consistent with other known inputs to the putamen (Smith et al., Journal of Neurophsyiology, 2012; Weiss et al., Scientific Reports, 2020).

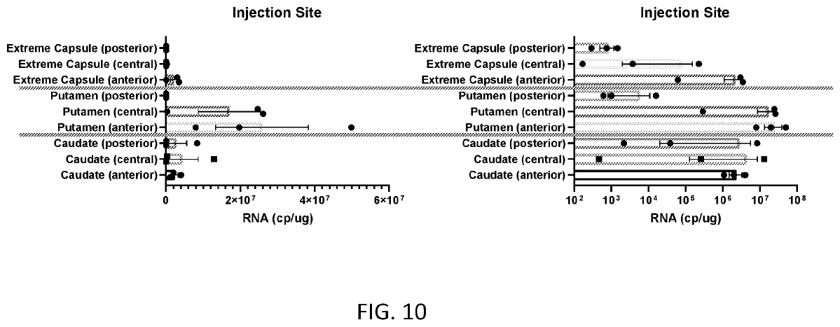

FIG. 10 depicts on overview of the relationship between vector spread from posterior compartments to anterior compartments, as an absolute measure of RNA copy number vs. log scale. Localization was observed in the putamen, as well as the caudate regions. Vector expression was strongest in injection site (e.g. putamen anterior).

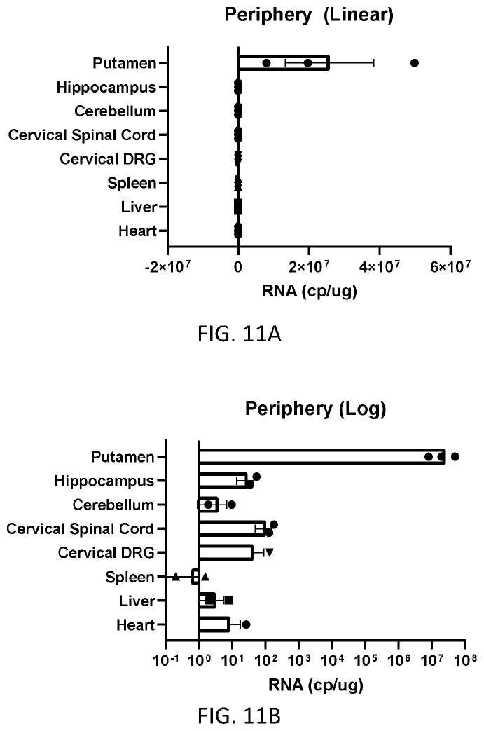

FIGS. 11A and 11B depict absolute RNA copy number/ug distribution by qPCR (A) and results adjusted to log scale (B), including peripheral tissues. Expression in the periphery and in unrelated brain regions was negligible, which comports with known lack of anatomical connectivity from cerebellum or hippocampus to putamen.

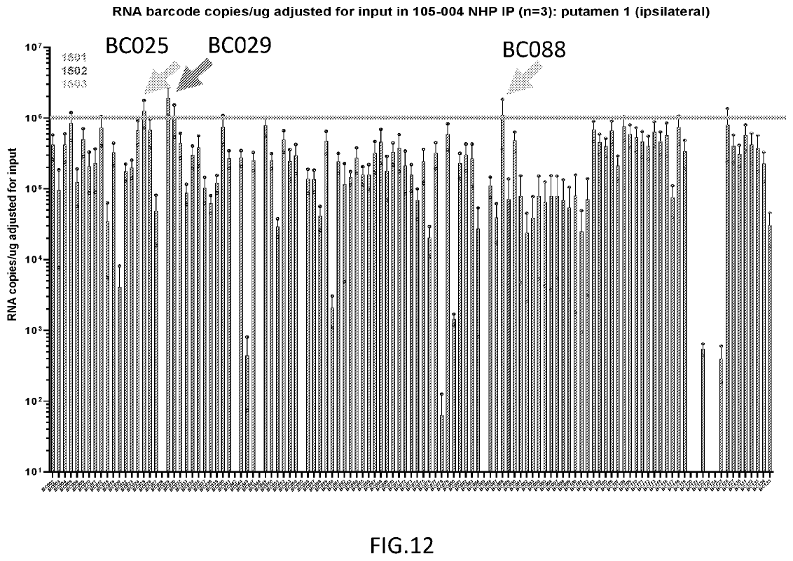

FIG. 12 depicts NGS analysis to identify vector DNA from the capsid pool in putamen. The BC029 barcode indicates the modified AAV9 capsid, AAV9.S454.Tfr3 is the highest transduced capsid identified in the putamen ipsilateral (relative to the location of injection) of this particular injected subject.

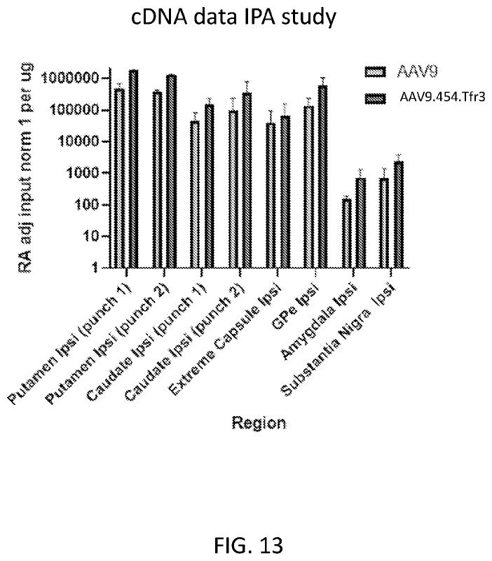

FIG. 13 depicts RNA abundance adjusted to input (normalized to 1 per μg). This is a representation of transcripts containing the barcode of interest per μg calculated using the barcode relative abundance adjusted for input normalized to 1 and total number of library-derived transcripts per μg in that tissue. qPCR was performed to identify vector transcripts relative to high expressing capsids in the selected tissues: putamen ipsilateral (sample “punch” 1 or 2), caudate ipsilateral (punch 1 or 2), extreme capsule ipsilateral, GPe (globus pallidus external) ipsilateral, amygdala ipsilateral, substantia nigra ipsilateral. AAV9.S454.Tfr3 demonstrates improved transduction relative to AAV9 following intraparenchymal delivery, for example a 4 to 5 fold increase RNA expression compared to AAV9 is observed in most brain regions analyzed.

In a biodistribution experiment in mice, NGS analysis of brain gDNA from mice to which capsid pools were delivered following IV injection was used to assess relative abundances (percent composition) of the capsids in the pool. AAV9.S454.Tfr3 (BC029) had a relative abundance of about.3 as compared to AAV9 which had a relative abundance of about 0.08. The data was normalized based on the composition of AAVs in the originally injected pool and quantified using the total genome copy number obtained from qPCR analysis with a primer-probe combination specific to the eGFP sequence (data not shown).

However, in the analogous study administering pooled barcoded vectors IV to NHPs, AAV9.454.Tfr3 is biodistributed to peripheral tissues similarly to AAV9 yet AAV9.454.Tfr3 is diminished in brain regions compared to AAV9. The same trend is seen in mouse brains following IV administration. The data suggests that AAV9.454.Tfr3 is less efficient at transport across the blood-brain-barrier than AAV9 following an IV administration of pooled capsids.

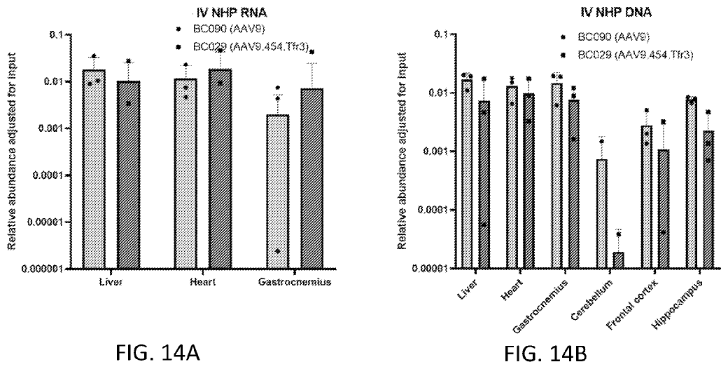

FIGS. 14A and 14B show biodistribution of AAV9 and AAV9.454. Tfr3 vector RNA and DNA following IV administration of pooled capsids intravenously in NHPs.

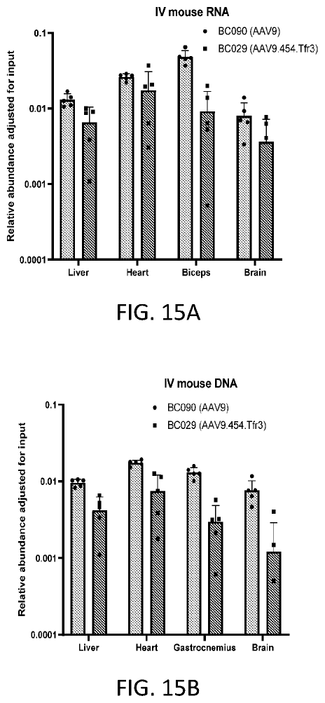

FIGS. 15A and 15B illustrate the biodistribution of AAV9 and AAV9.454.Tfr3 vector RNA and DNA in selected tissues following IV administration of pooled capsids intravenously in mice.

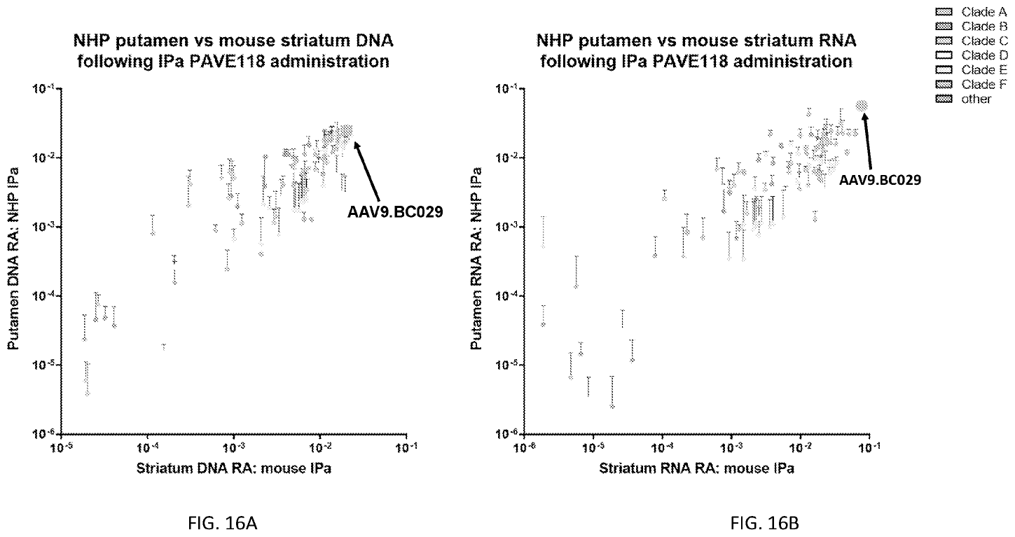

FIGS. 16A and 16B illustrate the correlation between abundances of transgene DNA and transcribed transgene RNA in NHP and mouse in the putamen (NHP) or striatum (mouse) following intraparenchymal delivery to striatum (mouse) or putamen (NHP). AAV9.454.TFR3 identified as top hit in both NHP and mouse experiments and findings translated well between mouse and primate at both the DNA and RNA levels.

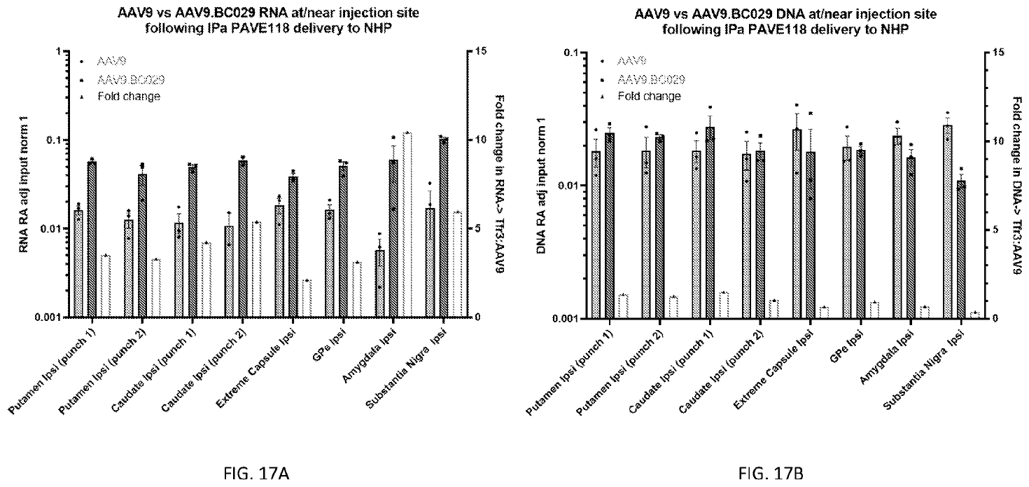

FIGS. 17A and 17B illustrate that rAAVs having an AAV9.S454.Tfr3 capsid produces 2-10 fold more transgene RNA than an rAAV with an AAV9 capsid with similar biodistribution following intraparenchymal administration in NHPs. A difference was observed in RNA relative abundance (RA) for AAV9.S454.Tfr3 compared to AAV9 (AAV9.S454.Tfr3>AAV9 by 2-10 fold) which was not seen at the DNA level.

FIGS. 18A and 18B illustrate that rAAV having an AAV9.S454. Tfr3 capsid produces 2-10 fold more RNA than AAV9 with similar biodistribution following intraparenchymal administration in mouse.

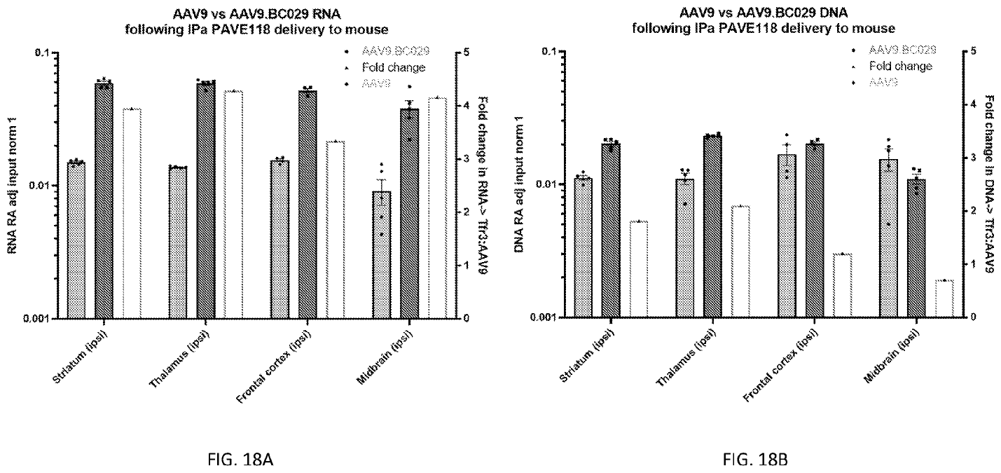

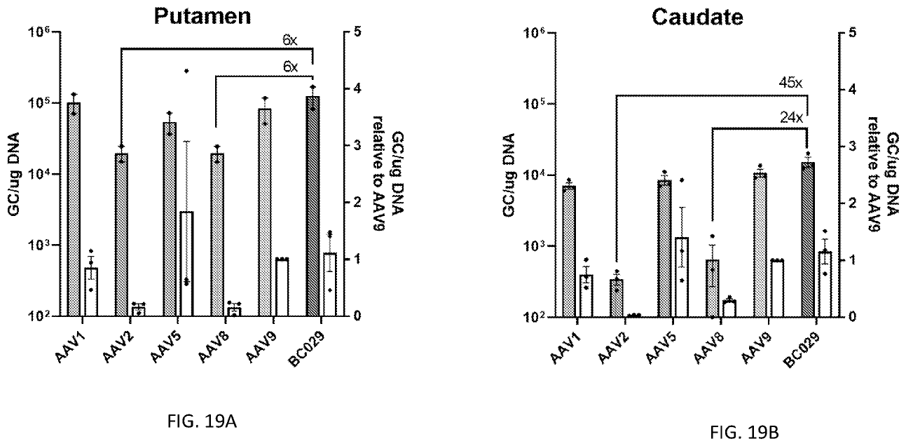

Further analysis of the intraparenchymal administration of the PAVE118 library (2.4e11GC in 100 μl) to NHP putamen is depicted in FIGS. 51A and 51B. GC/ug DNA was calculated from the relative abundance of each library member and the qPCR total biodistribution data in 3 tissue punches each from putamen (FIG. 19A) and caudate (FIG. 19B) from the ipsilateral hemisphere. BC029 (AAV9S454-TFR3) genome copy (GC) levels were determined to be equivalent to those of AAV9, AAV5 and AAV1. The fold change in genome copy (GC/ug DNA) for each capsid, normalized to AAV9, is depicted in white bars on the right y-axis.

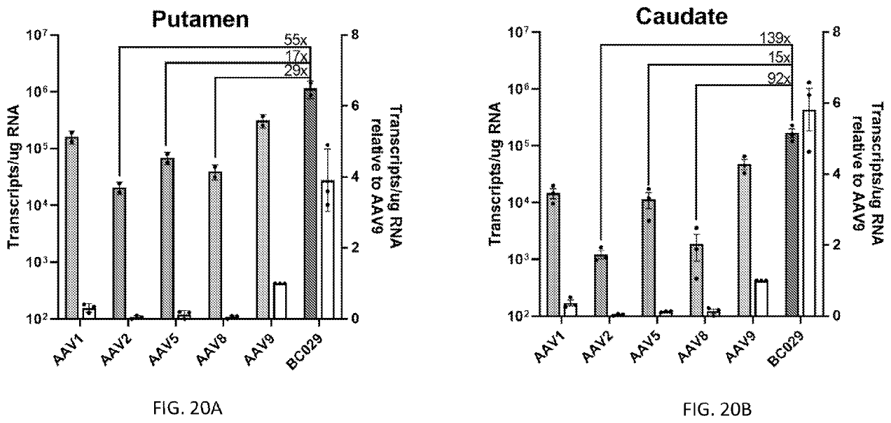

In the NHPs administered the PAVE118 library intraparenchymally, BC029 (AAV9S454-TFR3) results in higher RNA expression levels than does AAV9 at or near the injection site in putamen (FIG. 20A) and caudate (FIG. 20B). RNA expression levels (transcripts/ug RNA) produced from each capsid were calculated (left Y-axis). The fold change in expression level (transcripts/ug RNA) for each capsid, normalized to AAV9, is depicted in white bars on the right y-axis. A 4-fold improvement in putamen and nearly 6-fold improvement in caudate of BC029 (AAV9S454-TFR3) was observed over transcription from AAV9, which in resulted in further improvement of BC029 over AAV2, AAV5, and AAV8 as compared to AAV9.

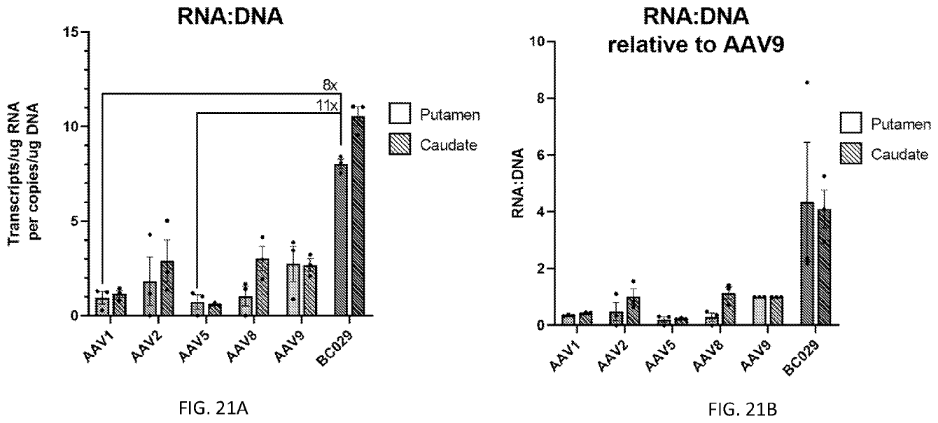

The superior transduction efficiency for AAV9S454-TFR3 can be attributed to an improved RNA: DNA ratio. Taking the DNA and RNA analysis together (transcripts/ug RNA per copies/ug DNA), AAV9S454-TFR3 (BC029) produces approximately 4 fold more RNA per genome copy than does AAV9 in both putamen (solid bars) and caudate (hatched bars) (FIG. 21A). The ratio of RNA expression levels to DNA genome copy number, normalized to AAV9, in putamen (solid bars) and caudate (hatched bars) is shown in FIG. 21B.

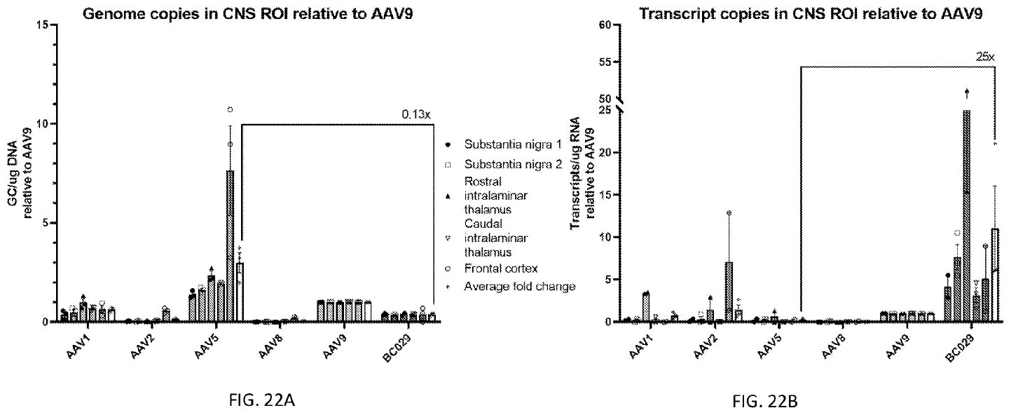

Additionally, tissue punches were analyzed in central nervous system (CNS) regions of interest (ROI), including two regions of the substantia nigra (substantia nigra 1 and substantia nigra 2), the rostral intralaminar thalamus, the caudal intralaminar thalamus and the frontal cortex. FIG. 22A shows the genome copies (GC/ug DNA) for AAV9S454-TFR3 (BC029) relative to AAV9 in the CNS regions sampled. AAV9S454-TFR3 (BC029) has, on average, 40% of the number of genome copies of AAV9 and 10% of the number of genome copies of AAV5. FIG. 22B shows RNA expression levels of AAV9S454-TFR3 (BC029) relative to AAV9 in the CNS regions sampled. AAV9S454-TFR3 (BC029) expresses 3-25 times more RNA than AAV9. The average fold change across these regions is depicted by the white bars. Extensive sampling across the entire brain was performed and BC029 consistently outperformed AAV9, driven by an increase in RNA production (data not shown).

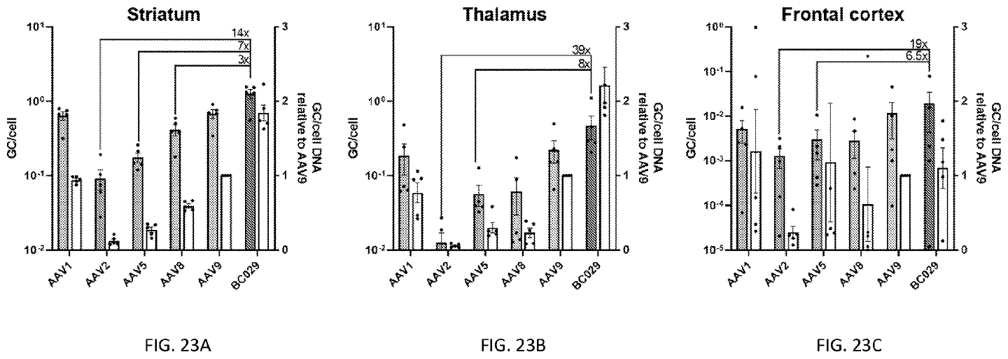

Similar assessment was performed in mice by administration of the PAVE118 library (7.22e9 in 1 μl) by unilateral stereotaxic injection to mouse striatum. Tissues were harvested three weeks after administration for analysis. GC/cell (left y-axis) of AAV9S454-TFR3 (BC029) and GC/cell DNA of AAV9S454-TFR3 relative to AAV9 (right y-axis) was analyzed in the striatum (FIG. 23A), thalamus (FIG. 23B) and frontal cortex (FIG. 23C). There is approximately a two-fold increase in BC029 genome copies compared to AAV9 genome copies in the thalamus. BC029 biodistribution was higher than other AAV serotypes evaluated in striatum, thalamus and frontal cortex.

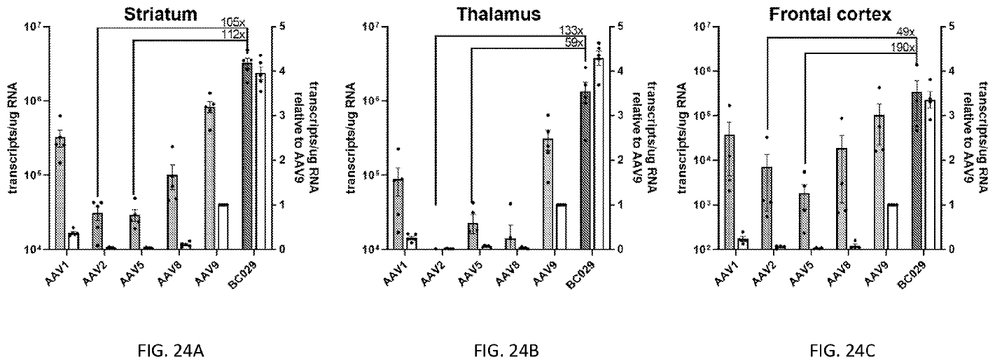

BC029 produces 3-4 fold more RNA transcripts than AAV9 in the mouse striatum (FIG. 24A), thalamus (FIG. 24B) and frontal cortex (FIG. 24C). RNA expression levels (transcripts/ug RNA) produced from each capsid were calculated (left Y-axis). The fold change in expression level (transcripts/ug RNA) for each capsid, normalized to AAV9, is depicted in white bars on the right y-axis. BC029 produces significantly higher transcript levels than reference AAV serotypes by approximately 1-2 logs.

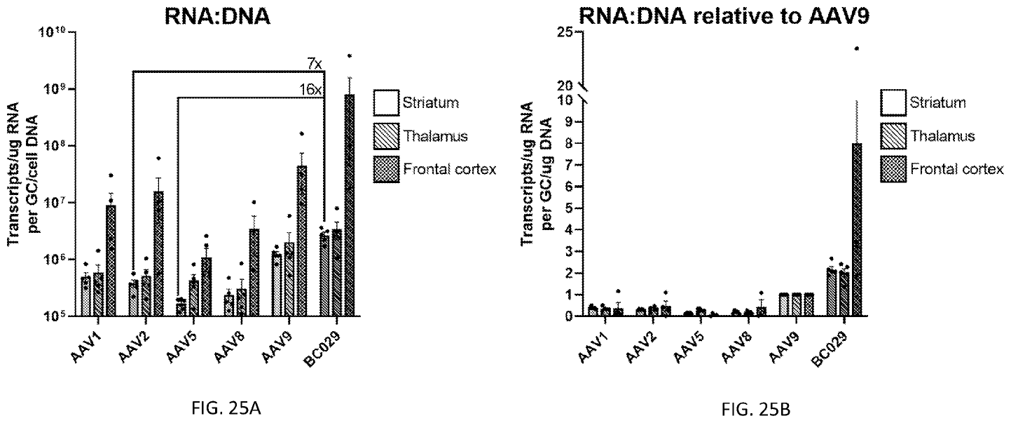

Thus, BC029 (AAV9S454-TFR3) has approximately two-fold higher transcriptional activity than AAV9 in striatum and thalamus relative to AAV9 in mice administered AAV particles to the striatum. RNA: DNA ratios, absolute (FIG. 25A) and relative to AAV9 (FIG. 25B) in striatum (solid bars) thalamus (hatched bars) and frontal cortex (checked bars) were calculated as described above. The RNA: DNA ratio of BC029 was improved by only 2 fold in striatum and thalamus relative to AAV9 due to increase in BC029 GC/cell (this increase was not observed in these regions in NHP as shown above). This difference trended higher in the cortex, but this was largely driven by a single animal.

Taken together, these data in NHP and mice indicate that BC029 (AAV9S454-TFR3) displays improved transduction over AAV9 in multiple species via multiple routes of administration, largely doing so by producing more transcript copies than does AAV9 from equivalent genome copy levels in regions of interest in the CNS.

### 6.20. Example 10 Evaluation of the Cell Type Specificity of AAV Variants in Brain

To evaluate both the transduction efficiency as well as cell type specificity of transduction, vector preparations of AAV9, AAV9.Tfr3 (AAV9-TfR peptide, RTIGPSV, SEQ ID NO: 12; capsid sequence SEQ ID NO:42), and AAV9.Ref (AAV9-Reference peptide) were produced using 2 distinct sets of cis plasmids, and therefore packaging 2 distinct sets of vector genomes. First, a vector genome expressing a unique fluorescent reporter, either GFP, tdTomato, or iRFP670, under the control of the universal CAG reporter and including a unique 20 bp barcode between the fluorescent reporter coding sequence and the polyadenylation signal, was packaged into AAV9 (GFP), AAV9.TfR3 (tdTomato), or AAV9.Ref (iRFP) capsids. Second, a vector genome expressing a codon-optimized, human ApoE transgene, under the control of the universal CAG reporter and containing a unique 20 bp barcode between the ApoE coding sequence and the polyadenylation signal (a different set of barcodes than those used in the fluorescent reporter cassettes) was packaged into AAV9, AAV9.TfR3, or AAV9.Ref such that ApoE transcripts produced from one of these capsids could be attributed to a transduction event with that capsid.

Following titering of each vector prep by ddPCR using a primer/probe set specific for the poly A sequence, the titer of each fluorescent reporter prep was used to calculate the volume of each prep required to formulate a test article with equimolar concentrations of AAV9.CAG.GFP, AAV9.TfR3.CAG.tdTomato, and AAV9.Ref.CAG.iRFP, and vector was formulated accordingly. Similarly, the ddPCR titer of each ApoE reporter prep was used to calculate the volume of each prep required to formulate a test article with equimolar concentrations of AAV9.CAG.hcoApoE.BC1, AAV9.TfR3.hcoApoE.BC4, and AAV9.Ref.hcoApoE.BC5, and vector was formulated accordingly. Finally, these 2 vector pools were pooled such that 90% of total GC in the final preparation were derived from fluorescent reporter preps, with the 10% of contributing GC being from ApoE reporter preps. As such, final “Tricolor-2” formulated vector pool had a titer of 2e13 GC/mL, with each fluorescent reporter prep having an effective concentration of 6e12 GC/mL and each ApoE reporter prep having an effective concentration of 6.6e11 GC/mL.

The pooled Tricolor-2 test article was administered intraparenchymally to adult, cynomolgus macaques by MRI-guided delivery, following a bilateral dosing scheme into both the hippocampus (60 ul of 2e13 GC/mL test article, 1.2e12 GC total) and the putamen (75 uL of 2e13 GC/mL test article, 1.5e12 GC total). A custom 16 gauge, 10 ft. SmartFlow Neuro Ventricular Cannula was used, and Prohance was added prior to cannula fill in order to allow for repeated T1-weighted MRI monitoring of dosing solution delivery. The cannula was first primed with dosing solution, followed by dosing solution hold in the cannula for at least 5m to control for any vector adsorption to the device. Dosing solution was then eluted, followed by adjustment of flow rate to 2-5 uL/min, cannula was inserted into target region, flow rate reduced to luL/min until complete dose was delivered. Animals were sacrificed 3 weeks post-test article delivery, and brain was collected in 3 mm thick coronal sections; alternating sections were fixed for histological analysis and direct visualization of fluorescent reporter expression or were sampled using a 4 mm punch for nucleic acid analysis. Additional tissues were also collected for nucleic acid analysis. From each punch, DNA and RNA were extracted using a custom workflow such that paired DNA-RNA data could be obtained from the same tissue sample. The DNA and RNA (following conversion to cDNA) were then subjected to amplicon sequencing using the Illumina MiSeq platform after amplification of barcode-containing regions from nucleic acids derived both from the fluorescent and ApoE reporters. Following sequencing, barcode counts were adjusted for test article input concentrations and normalized to 1, such that the fraction of total fluorescent reporter or total ApoE reporter DNA or RNA that was derived from one of the 3 capsids of interest could be determined. Similarly the pooled vector cocktail was administered to the striatum of mice (1.2e10GC total in putamen in 2 μl) and mice were sacrificed 3 weeks after administration and tissue samples analyzed.

There was significant overlap in cells transduced by AAV9 and BC029 in both the striatum of mice and the putamen of NHP (data not shown). Like AAV9, BC029 transduces multiple cell types present in the striatum, including those of neural and glial origin. There was also significant overlap in cells transduced by AAV9 and BC029 in the cortex following delivery of the pooled vector to NHP putamen (1.5e12 GC total/75 μl) (data not shown). Following delivery of the pooled vector to NHP hippocampus (1.2e12 GC total into hippocampus in 60 μl), BC029 and AAV display similar tropism for hippocampal regions of interest (ROIs) (dentate gyrus, CA3, CA2 and subiculum regions of the hippocampus) (data not shown). In the dentate gyrus, BC029 and AAV9 similarly transduce the polymorphic and granule cell layers (data not shown).

### 6.21. Example 11 Evaluation of Intrathecal Delivery (ICV) of PAVE118 Library

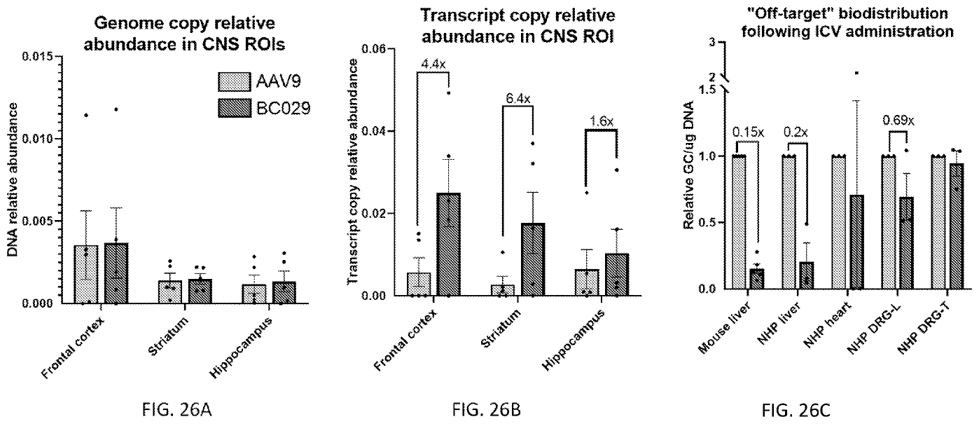

PAVE118 library was administered by ICV infusion to the left ventricle in mice (1.44e10 GC/brain) and NHP (3e10 GC/g brain). Genome copy (FIG. 26A) and RNA transcript abundance (FIG. 26B) were measured in CNS ROI (frontal cortex, striatum and hippocampus). Genome copy was equivalent between BC029 and AAV9, however, the relative abundance of RNA transcripts was up to 6.4 fold higher in BC029 compared to AAV9. Off-target biodistribution (GC/ug DNA) was analyzed in mouse liver, NHP liver, NHP heart, NHP lumbar DRG and NHP thoracic DRG (FIG. 26C). In both mouse and NHP liver, BC029 genome copies were approximately 5 fold lower than AAV9 genome copies. There was approximately a 30% decline in BC029 genome copies in lumbar DRG compared to AAV genome copies. BC029 and AAV9 GC levels were equivalent in thoracic DRG. In conclusion, improved transduction profile of BC029 relative to AAV9 is also seen in ICV administration. Finally, due to a more favorable transduction profile in off-target tissues, use of BC029 has the potential to ameliorate concerns surrounding undesired peripheral tissue transduction.

### 6.23 Capsid Amino Acid Sequences

Table 4 provides the amino acid sequences of certain engineered capsid proteins described and/or used in studies described herein. Heterologous peptides and amino acid substitutions are indicated in gray shading.

TABLE 4
Acid Sequences

| | Insert or | | |
|---|---|---|---|
| Capsid | Sub- | | |
| Name | stitution | Amino Acid Sequence | |
| PHP.S | QAVRT | 1 | MAADGYLPDW LEDNLSEGIR EWWALKPGAP QPKANQQHQD NARGLVLPGY KYLGPGNGLD |
| (California | SL (SEQ | 61 | KGEPVNAADA AALEHDKAYD QQLKAGDNPY LKYNHADAEF QERLKEDTSF GGNLGRAVFQ |
| Institute | ID | 121 | AKKRLLEPLG LVEEAAKTAP GKKRPVEQSP QEPDSSAGIG KSGAQPAKKR LNFGQTGDTE |
| of | NO: 16) | 181 | SVPDPQPIGE PPAAPSGVGS LTMASGGGAP VADNNEGADG VGSSSGNWHC DSQWLGDRVI |
| Technology- | (588_589) | 241 | TTSTRTWALP TYNNHLYKQI SNSTSGGSSN DNAYFGYSTP WGYFDENRFH CHFSPRDWQR |
| Chan et | | 301 | LINNNWGFRP KRLNFKLFNI QVKEVTDNNG VKTIANNLTS TVQVFTDSDY QLPYVLGSAH |
| al 2017) | | 361 | EGCLPPFPAD VFMIPQYGYL TLNDGSQAVG RSSFYCLEYF PSQMLRTGNN FQFSYEFENV |
| | | 421 | PFHSSYAHSQ SLDRLMNPLI DQYLYYLSRT INGSGQNQQT LKFSVAGPSN MAVQGRNYIP |
| | | 481 | GPSYRQQRVS TTVTQNNNSE FAWPGASSWA LNGRNSLMNP GPAMASHKEG EDRFFPLSGS |
| | | 541 | LIFGKQGTGR DNVDADKVMI TNEEEIKTTN PVATESYGQV ATNHQSAQQA VRTSLAQAQT |
| | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 22) |
| PHP.SH | QAVRT | 1 | MAADGYLPDW LEDNLSEGIR EWWALKPGAP QPKANQQHQD NARGLVLPGY KYLGPGNGLD |
| | SH | 61 | KGEPVNAADA AALEHDKAYD QQLKAGDNPY LKYNHADAEF QERLKEDTSF GGNLGRAVFQ |
| | (SEQ | 121 | AKKRLLEPLG LVEEAAKTAP GKKRPVEQSP QEPDSSAGIG KSGAQPAKKR LNFGQTGDTE |
| | ID | 181 | SVPDPQPIGE PPAAPSGVGS LTMASGGGAP VADNNEGADG VGSSSGNWHC DSQWLGDRVI |
| | NO: 17) | 241 | TTSTRTWALP TYNNHLYKQI SNSTSGGSSN DNAYFGYSTP WGYFDENRFH CHFSPRDWQR |
| | (588_589) | 301 | LINNNWGFRP KRLNFKLFNI QVKEVTDNNG VKTIANNLTS TVQVFTDSDY QLPYVLGSAH |
| | | 361 | EGCLPPFPAD VFMIPQYGYL TLNDGSQAVG RSSFYCLEYF PSQMLRTGNN FQFSYEFENV |
| | | 421 | PFHSSYAHSQ SLDRLMNPLI DQYLYYLSRT INGSGQNQQT LKFSVAGPSN MAVQGRNYIP |
| | | 481 | GPSYRQQRVS TTVTQNNNSE FAWPGASSWA LNGRNSLMNP GPAMASHKEG EDRFFPLSGS |
| | | 541 | LIFGKQGTGR DNVDADKVMI TNEEEIKTTN PVATESYGQV ATNHQSAQQA VRTSHAQAQT |
| | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 23) |
| PHP.B | TLAVPF | 1 | MAADGYLPDW LEDNLSEGIR EWWALKPGAP QPKANQQHQD NARGLVLPGY KYLGPGNGLD |
| (California | K (SEQ | 61 | KGEPVNAADA AALEHDKAYD QQLKAGDNPY LKYNHADAEF QERLKEDTSF GGNLGRAVFQ |
| Institute | ID | 121 | AKKRLLEPLG LVEEAAKTAP GKKRPVEQSP QEPDSSAGIG KSGAQPAKKR LNFGQTGDTE |
| of | NO: 20) | 181 | SVPDPQPIGE PPAAPSGVGS LTMASGGGAP VADNNEGADG VGSSSGNWHC DSQWLGDRVI |
| Technology | (588_589) | 241 | TTSTRTWALP TYNNHLYKQI SNSTSGGSSN DNAYFGYSTP WGYFDENRFH CHFSPRDWQR |
| GenBank | | 301 | LINNNWGFRP KRLNFKLFNI QVKEVTDNNG VKTIANNLTS TVQVFTDSDY QLPYVLGSAH |
| entry: | | 361 | EGCLPPFPAD VFMIPQYGYL TLNDGSQAVG RSSFYCLEYF PSQMLRTGNN FQFSYEFENV |
| ALU85156.1- | | 421 | PFHSSYAHSQ SLDRLMNPLI DQYLYYLSRT INGSGQNQQT LKFSVAGPSN MAVQGRNYIP |
| Deverman | | 481 | GPSYRQQRVS TTVTQNNNSE FAWPGASSWA LNGRNSLMNP GPAMASHKEG EDRFFPLSGS |
| et al | | 541 | LIFGKQGTGR DNVDADKVMI TNEEEIKTTN PVATESYGQV ATNHQSAQTL AVPFKAQAQT |
| 2016) | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 24) |
| PHP.eB | TLAVP | 1 | MAADGYLPDW LEDNLSEGIR EWWALKPGAP QPKANQQHQD NARGLVLPGY KYLGPGNGLD |
| (California | FK | 61 | KGEPVNAADA AALEHDKAYD QQLKAGDNPY LKYNHADAEF QERLKEDTSF GGNLGRAVFQ |
| Institute | (SEQ ID | 121 | AKKRLLEPLG LVEEAAKTAP GKKRPVEQSP QEPDSSAGIG KSGAQPAKKR LNFGQTGDTE |
| of | NO: 20) | 181 | SVPDPQPIGE PPAAPSGVGS LTMASGGGAP VADNNEGADG VGSSSGNWHC DSQWLGDRVI |
| Technology- | (588_589) | 241 | TTSTRTWALP TYNNHLYKQI SNSTSGGSSN DNAYFGYSTP WGYFDFNRFH CHFSPRDWQR |
| Chan et | | 301 | LINNNWGFRP KRLNFKLFNI QVKEVTDNNG VKTIANNLTS TVQVFTDSDY QLPYVLGSAH |
| al 2017) | | 361 | EGCLPPFPAD VFMIPQYGYL TLNDGSQAVG RSSFYCLEYF PSQMLRTGNN FQFSYEFENV |
| | | 421 | PFHSSYAHSQ SLDRLMNPLI DQYLYYLSRT INGSGQNQQT LKFSVAGPSN MAVQGRNYIP |
| | | 481 | GPSYRQQRVS TTVTQNNNSE FAWPGASSWA LNGRNSLMNP GPAMASHKEG EDRFFPLSGS |
| | | 541 | LIFGKQGTGR DNVDADKVMI TNEEEIKTTN PVATESYGQV ATNHQSDGTL AVPFKAQAQT |
| | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 25) |

| | | | |
|---|---|---|---|
| AAV8.BBB | A269S | MAADGYLPDW LEDNLSEGIR EWWALKPGAP KPKANQQKQD DGRGLVLPGY KYLGPENGLD | 60 |
| | | KGEPVNAADA AALEHDKAYD QQLQAGDNPY LRYNHADAEF QERLQEDTSF GGNLGRAVEQ | 120 |
| | | AKKRVLEPLG LVEEGAKTAP GKKRPVEPSP QRSPDSSTGI GKKGQQPARK RLNFGQTGDS | 180 |
| | | ESVPDPQPLG EPPAAPSGVG PNTMAAGGGA PMADNNEGAD GVGSSSGNWH CDSTWLGDRV | 240 |
| | | ITTSTRTWAL PTYNNHLYKQ ISNGTSGGST NDNTYFGYST PWGYFDENRF HCHFSPRDWQ | 300 |
| | | RLINNNWGFR PKRLSFKLEN IQVKEVTQNE GTKTIANNLT STIQVFTDSE YQLPYVLGSA | 360 |
| | | HQGCLPPFPA DVFMIPQYGY LTLNNGSQAV GRSSFYCLEY FPSQMLRTGN NFQFTYTFED | 420 |
| | | VPFHSSYAHS QSLDRLMNPL IDQYLYYLSR TQTTGGTANT QTLGFSQGGP NTMANQAKNW | 480 |
| | | LPGPCYRQQR VSTTTGQNNN SNFAWTAGTK YHLNGRNSLA NPGIAMATHK DDEERFFPSN | 540 |
| | | GILIFGKQNA ARDNADYSDV MLTSEEEIKT TNPVATEEYG IVADNLQQQN TAPQIGTVNS | 600 |
| | | QGALPGMVWQ NRDVYLQGPI WAKIPHTDGN FHPSPLMGGF GLKHPPPQIL IKNTPVPADP | 660 |
| | | PTTFNQSKLN SFITQYSTGQ VSVEIEWELQ KENSKRWNPE IQYTSNYYKS TSVDFAVNTE | 720 |
| | | GVYSEPRPIG TRYLTRNL (SEQ ID NO: 26) | |
| AAV8.BBB. | A269S, | MAADGYLPDW LEDNLSEGIR EWWALKPGAP KPKANQQKQD DGRGLVLPGY KYLGPENGLD | 60 |
| LD | 498_NNN/ | KGEPVNAADA AALEHDKAYD QQLQAGDNPY LRYNHADAEF QERLQEDTSF GGNLGRAVFQ | 120 |
| | AAA_500 | AKKRVLEPLG LVEEGAKTAP GKKRPVEPSP QRSPDSSTGI GKKGQQPARK RLNFGQTGDS | 180 |
| | | ESVPDPQPLG EPPAAPSGVG PNTMAAGGGA PMADNNEGAD GVGSSSGNWH CDSTWLGDRV | 240 |
| | | ITTSTRTWAL PTYNNHLYKQ ISNGTSGGST NDNTYFGYST PWGYFDENRF HCHFSPRDWQ | 300 |
| | | RLINNNWGFR PKRLSFKLEN IQVKEVTQNE GTKTIANNLT STIQVFTDSE YQLPYVLGSA | 360 |
| | | HQGCLPPFPA DVFMIPQYGY LTINNGSQAV GRSSFYCLEY FPSQMLRTGN NFQFTYTFED | 420 |
| | | VPFHSSYAHS QSLDRLMNPL IDQYLYYLSR TQTTGGTANT QTLGFSQGGP NTMANQAKNW | 480 |
| | | LPGPCYRQQR VSTTTGQAAA SNFAWTAGTK YHLNGRNSLA NPGIAMATHK DDEERFFPSN | 540 |
| | | GILIFGKQNA ARDNADYSDV MLTSEEEIKT TNPVATEEYG IVADNLQQQN TAPQIGTVNS | 600 |
| | | QGALPGMVWQ NRDVYLQGPI WAKIPHTDGN FHPSPLMGGF GLKHPPPQIL IKNTPVPADP | 660 |
| | | PTTFNQSKLN SFITQYSTGQ VSVEIEWELQ KENSKRWNPE IQYTSNYYKS TSVDFAVNTE | 720 |
| | | GVYSEPRPIG TRYLTRNL (SEQ ID NO: 27) | |
| AAV9.BBB | S263G/ | MAADGYLPDW LEDNLSEGIR EWWALKPGAP QPKANQQHQD NARGLVLPGY KYLGPGNGLD | 60 |
| | S269T/ | KGEPVNAADA AALEHDKAYD QQLKAGDNPY LKYNHADAEF QERLKEDTSF GGNLGRAVFQ | 120 |
| | A273T | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNGTSGGSTN DNTYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 28) | |
| AAV9.BBB. | S263G/ | MAADGYLPDW LEDNLSEGIR EWWALKPGAP QPKANQQHQD NARGLVLPGY KYLGPGNGLD | 60 |
| LD | S269T/ | KGEPVNAADA AALEHDKAYD QQLKAGDNPY LKYNHADAEF QERLKEDTSF GGNLGRAVFQ | 120 |
| | A273T, | AKKRLLEPLG LVEEAAKTAP GKKRPVEQSP QEPDSSAGIG KSGAQPAKKR LNFGQTGDTE | 180 |
| | 496_NNN/ | SVPDPQPIGE PPAAPSGVGS LTMASGGGAP VADNNEGADG VGSSSGNWHC DSQWLGDRVI | 240 |
| | AAA_498 | TTSTRTWALP TYNNHLYKQI SNGTSGGSTN DNTYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 29) | |
| AAVrh.10. | 498_NNN/ | MAADGYLPDW LEDNLSEGIR EWWDLKPGAP KPKANQQKQD DGRGLVLPGY | 50 |
| LD | AAA_500 | KYLGPFNGLD KGEPVNAADA AALEHDKAYD QQLKAGDNPY LRYNHADAEF | 100 |
| | | QERLQEDTSF GGNLGRAVFQ AKKRVLEPLG LVEEGAKTAP GKKRPVEPSP | 150 |
| | | QRSPDSSTGI GKKGQQPAKK RLNFGQTGDS ESVPDPQPIG EPPAGPSGLG | 200 |
| | | SGTMAAGGGA PMADNNEGAD GVGSSSGNWH CDSTWLGDRV ITTSTRTWAL | 250 |
| | | PTYNNHLYKQ ISNGTSGGST NDNTYFGYST PWGYFDENRF HCHFSPRDWQ | 300 |
| | | RLINNNWGFR PKRLNFKLEN IQVKEVTQNE GTKTIANNLT STIQVFTDSE | 350 |
| | | YQLPYVLGSA HQGCLPPFPA DVFMIPQYGY LTLNNGSQAV GRSSFYCLEY | 400 |
| | | FPSQMLRTGN NFEFSYQFED VPFHSSYAHS QSLDRLMNPL IDQYLYYLSR | 450 |
| | | TQSTGGTAGT QQLLFSQAGP NNMSAQAKNW LPGPCYRQQR VSTTLSQAAA | 500 |
| | | SNFAWTGATK YHLNGRDSLV NPGVAMATHK DDEERFFPSS GVLMFGKQGA | 550 |
| | | GKDNVDYSSV MLTSEEEIKT TNPVATEQYG VVADNLQQQN AAPIVGAVNS | 600 |
| | | QGALPGMVWQ NRDVYLQGPI WAKIPHTDGN FHPSPLMGGF GLKHPPPQIL | 650 |
| | | IKNTPVPADP PTTFSQAKLA SFITQYSTGQ VSVEIEWELQ KENSKRWNPE | 700 |
| | | IQYTSNYYKS TNVDFAVNTD GTYSEPRPIG TRYLTRNL (SEQ ID NO: 30) | |
| AAV9. | 498_NNN/ | MAADGYLPDW LEDNLSEGIR EWWALKPGAP QPKANQQHQD NARGLVLPGY KYLGPGNGLD | 60 |
| 496NNN/ | AAA_500 | KGEPVNAADA AALEHDKAYD QQLKAGDNPY LKYNHADAEF QERLKEDTSF GGNLGRAVFQ | 120 |
| AAA498 | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 31) | |
| AAV9. | 496NNN/ | MAADGYLPDW LEDNLSEGIR EWWALKPGAP QPKANQQHQD NARGLVLPGY KYLGPGNGLD | 60 |
| 496NNN/ | AAA498, | KGEPVNAADA AALEHDKAYD QQLKAGDNPY LKYNHADAEF QERLKEDTSF GGNLGRAVFQ | 120 |
| AAA498. | W503R | AKKRLLEPLG LVEEAAKTAP GKKRPVEQSP QEPDSSAGIG KSGAQPAKKR LNFGQTGDTE | 180 |
| W503R | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FARPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 32) | |
| AAV9 | W503R | MAADGYLPDW LEDNLSEGIR EWWALKPGAP QPKANQQHQD NARGLVLPGY KYLGPGNGLD | 60 |
| W503R | | KGEPVNAADA AALEHDKAYD QQLKAGDNPY LKYNHADAEF QERLKEDTSF GGNLGRAVEQ | 120 |
| | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FARPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 33) | |
| AAV9 | Q474A | MAADGYLPDW LEDNLSEGIR EWWALKPGAP QPKANQQHQD NARGLVLPGY KYLGPGNGLD | 60 |
| Q474A | | KGEPVNAADA AALEHDKAYD QQLKAGDNPY LKYNHADAEF QERLKEDTSF GGNLGRAVFQ | 120 |
| | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVAGRNYIP | 480 |
| | | GPSYRQQRVS TTVTQNNNSE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 34) | |
| AAV9 | Bone1, | MAADGYLPDW LEDNLSEGIR EWWALKPGAP QPKANQQHQD NARGLVLPGY KYLGPGNGLD | 60 |
| S454- | DDDDDDDD | KGEPVNAADA AALEHDKAYD QQLKAGDNPY LKYNHADAEF QERLKEDTSF GGNLGRAVFQ | 120 |
| D8 | (SEQ ID | AKKRLLEPLG LVEEAAKTAP GKKRPVEQSP QEPDSSAGIG KSGAQPAKKR LNFGQTGDTE | 180 |
| | NO: 2) | SVPDPQPIGE PPAAPSGVGS LTMASGGGAP VADNNEGADG VGSSSGNWHC DSQWLGDRVI | 240 |
| | (454_455 | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSDDDDDD DDGQNQQTLKF SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 35) | |
| AAV9 | Brain1, | MAADGYLPDW LEDNLSEGIR EWWALKPGAP QPKANQQHQD NARGLVLPGY KYLGPGNGLD | 60 |
| S454- | LSSRLDA | KGEPVNAADA AALEHDKAYD QQLKAGDNPY LKYNHADAEF QERLKEDTSF GGNLGRAVFQ | 120 |
| Brain1 | (SEQ ID | AKKRLLEPLG LVEEAAKTAP GKKRPVEQSP QEPDSSAGIG KSGAQPAKKR LNFGQTGDTE | 180 |
| | NO: 3) | SVPDPQPIGE PPAAPSGVGS LTMASGGGAP VADNNEGADG VGSSSGNWHC DSQWLGDRVI | 240 |
| | (454_455 | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSLSSRLD AGQNQQTLKF SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 36) | |
| AAV9 | Brain2/ | MAADGYLPDW LEDNLSEGIR EWWALKPGAP QPKANQQHQD NARGLVLPGY KYLGPGNGLD | 60 |
| S454- | Brain1C, | KGEPVNAADA AALEHDKAYD QQLKAGDNPY LKYNHADAEF QERLKEDTSF GGNLGRAVFQ | 120 |
| Brain2 | CLSSRLDAC | AKKRLLEPLG LVEEAAKTAP GKKRPVEQSP QEPDSSAGIG KSGAQPAKKR LNFGQTGDTE | 180 |
| | (SEQ ID | SVPDPQPIGE PPAAPSGVGS LTMASGGGAP VADNNEGADG VGSSSGNWHC DSQWLGDRVI | 240 |
| | NO: 4) | TTSTRTWALP TYNNHLYKQI SNSTSGGSSN DNAYFGYSTP WGYFDENRFH CHFSPRDWQR | 300 |
| | (454_455 | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSCLSSRL CACGQNQQTL KFSVAGPSNM | 482 |
| | | AV | |
| | | QGRNYIPGPS YRQQRVSTTV TQNNNSEFAW PGASSWALNG RNSLMNPGPA MASHKEGEDR | 542 |
| | | FFPLSGSLIF GKQGTGRDNV DADKVMITNE EEIKTTNPVA TESYGQVATN HQSAQAQAQT | 602 |
| | | GWVQNQGILP GMVWQDRDVY LQGPIWAKIP HTDGNFHPSP LMGGFGMKHP PPQILIKNTP | 662 |
| | | VPADPPTAFN KDKLNSFITQ YSTGQVSVEI EWELQKENSK RWNPEIQYTS NYYKSNNVEF | 722 |
| | | AVNTEGVYSE PRPIGTRYLT RNL (SEQ ID NO: 37) | |
| AAV9 | Kidney 1, | MAADGYLPDW LEDNLSEGIR EWWALKPGAP QPKANQQHQD NARGLVLPGY KYLGPGNGLD | 60 |
| S454- | LPVAS | KGEPVNAADA AALEHDKAYD QQLKAGDNPY LKYNHADAEF QERLKEDTSF GGNLGRAVFQ | 120 |
| Kidney | (SEQ ID | AKKRLLEPLG LVEEAAKTAP GKKRPVEQSP QEPDSSAGIG KSGAQPAKKR LNFGQTGDTE | 180 |
| 1 | NO: 6) | SVPDPQPIGE PPAAPSGVGS LTMASGGGAP VADNNEGADG VGSSSGNWHC DSQWLGDRVI | 240 |
| | (454_455 | TTSTRTWALP TYNNHLYKQI SNSTSGGSSN DNAYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSLPVASG QNQQTLKFSV AGPSNMAV | 478 |
| | | QGRNYIPGPS YRQQRVSTTV TQNNNSEFAW PGASSWALNG RNSLMNPGPA MASHKEGEDR | 538 |
| | | FFPLSGSLIF GKQGTGRDNV DADKVMITNE EEIKTTNPVA TESYGQVATN HQSAQAQAQT | 598 |
| | | GWVQNQGILP GMVWQDRDVY LQGPIWAKIP HTDGNFHPSP LMGGFGMKHP PPQILIKNTP | 658 |
| | | VPADPPTAFN KDKLNSFITQ YSTGQVSVEI EWELQKENSK RWNPEIQYTS NYYKSNNVEF | 718 |
| | | AVNTEGVYSE PRPIGTRYLT RNL (SEQ ID NO: 38) | |
| AAV9 | Kidney 2/ | MAADGYLPDW LEDNLSEGIR EWWALKPGAP QPKANQQHQD NARGLVLPGY KYLGPGNGLD | 60 |
| S454- | Kidney 1 | KGEPVNAADA AALEHDKAYD QQLKAGDNPY LKYNHADAEF QERLKEDTSF GGNLGRAVFQ | 120 |
| Kidney | C, | AKKRLLEPLG LVEEAAKTAP GKKRPVEQSP QEPDSSAGIG KSGAQPAKKR LNFGQTGDTE | 180 |
| 2 | CLPVASC | SVPDPQPIGE PPAAPSGVGS LTMASGGGAP VADNNEGADG VGSSSGNWHC DSQWLGDRVI | 240 |
| | (SEQ ID | TTSTRTWALP TYNNHLYKQI SNSTSGGSSN DNAYFGYSTP WGYFDENRFH CHFSPRDWQR | 300 |
| | NO: 5) | LINNNWGFRP KRLNFKLENI QVKEVTDNNG VKTIANNLTS TVQVFTDSDY QLPYVLGSAH | 360 |
| | (454_455 | EGCLPPFPAD VFMIPQYGYL TINDGSQAVG RSSFYCLEYF PSQMLRTGNN FQFSYEFENV | 420 |
| | | PFHSSYAHSQ SLDRLMNPLI DQYLYYLSKT INGSCLPVAS CGQNQQTLKF SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSPLMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 39) | |
| AAV9 | Muscle1, | MAADGYLPDW LEDNLSEGIR EWWALKPGAP QPKANQQHQD NARGLVLPGY KYLGPGNGLD | 60 |
| S454- | ASSLNIA | KGEPVNAADA AALEHDKAYD QQLKAGDNPY LKYNHADAEF QERLKEDTSF GGNLGRAVFQ | 120 |
| Muscle | (SEQ ID | AKKRLLEPLG LVEEAAKTAP GKKRPVEQSP QEPDSSAGIG KSGAQPAKKR LNFGQTGDTE | 180 |
| 1 | NO: 7) | SVPDPQPIGE PPAAPSGVGS LTMASGGGAP VADNNEGADG VGSSSGNWHC DSQWLGDRVI | 240 |
| | (454_455 | TTSTRTWALP TYNNHLYKQI SNSTSGGSSN DNAYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSASSLNI AGQNQQTLKF SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 40) | |
| AAV9 | Tfr1, | MAADGYLPDW LEDNLSEGIR EWWALKPGAP QPKANQQHQD NARGLVLPGY KYLGPGNGLD | 60 |
| S454- | HAIYPRH | KGEPVNAADA AALEHDKAYD QQLKAGDNPY LKYNHADAEF QERLKEDTSF GGNLGRAVFQ | 120 |
| Tfr1 | (SEQ ID | AKKRLLEPLG LVEEAAKTAP GKKRPVEQSP QEPDSSAGIG KSGAQPAKKR LNFGQTGDTE | 180 |
| | NO: 10) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSHAIYPR HGQNQQTLKF SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 41) | |
| AAV9 | Tfr3, | MAADGYLPDW LEDNLSEGIR EWWALKPGAP QPKANQQHQD NARGLVLPGY KYLGPGNGLD | 60 |
| S454- | RTIGPSV | KGEPVNAADA AALEHDKAYD QQLKAGDNPY LKYNHADAEF QERLKEDTSF GGNLGRAVFQ | 120 |
| Tfr3 | (SEQ ID | AKKRLLEPLG LVEEAAKTAP GKKRPVEQSP QEPDSSAGIG KSGAQPAKKR LNFGQTGDTE | 180 |
| | NO: 12) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSRTIGPS VGQNQQTLKF SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 42) | |
| AAV9 | Tfr4, | MAADGYLPDW LEDNLSEGIR EWWALKPGAP QPKANQQHQD NARGLVLPGY KYLGPGNGLD | 60 |
| S454- | CRTIGP | KGEPVNAADA AALEHDKAYD QQLKAGDNPY LKYNHADAEF QERLKEDTSF GGNLGRAVFQ | 120 |
| Tfr4 | SVC | AKKRLLEPLG LVEEAAKTAP GKKRPVEQSP QEPDSSAGIG KSGAQPAKKR LNFGQTGDTE | 180 |
| (AAV9 | (SEQ ID | SVPDPQPIGE PPAAPSGVGS LTMASGGGAP VADNNEGADG VGSSSGNWHC DSQWLGDRVI | 240 |
| S454- | NO: 13) | TTSTRTWALP TYNNHLYKQI SNSTSGGSSN DNAYFGYSTP WGYFDENRFH CHFSPRDWQR | 300 |
| TfR3C) | (454_455) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSCRTIGP SVCGQNQQTL KFSVAGPSNMAV | 482 |
| | | QGRNYIPGPS YRQQRVSTTV TQNNNSEFAW PGASSWALNG RNSLMNPGPA MASHKEGEDR | 542 |
| | | FFPLSGSLIF GKQGTGRDNV DADKVMITNE EEIKTTNPVA TESYGQVATN HQSAQAQAQT | 602 |
| | | GWVQNQGILP GMVWQDRDVY LQGPIWAKIP HTDGNFHPSP LMGGFGMKHP PPQILIKNTP | 622 |
| | | VPADPPTAFN KDKLNSFITQ YSTGQVSVEI EWELQKENSK RWNPEIQYTS NYYKSNNVEF | 722 |
| | | AVNTEGVYSE PRPIGTRYLT RNL (SEQ ID NO: 43) | |
| AAV9. | SITLVK | MAADGYLPDW LEDNLSEGIR EWWALKPGAP QPKANQQHQD NARGLVLPGY KYLGPGNGLD | 60 |
| 588Ad | STQTV | KGEPVNAADA AALEHDKAYD QQLKAGDNPY LKYNHADAEF QERLKEDTSF GGNLGRAVFQ | 120 |
| (9 588 | (SEQ ID | AKKRLLEPLG LVEEAAKTAP GKKRPVEQSP QEPDSSAGIG KSGAQPAKKR LNFGQTGDTE | 180 |
| Ad) | NO: 14), | SVPDPQPIGE PPAAPSGVGS LTMASGGGAP VADNNEGADG VGSSSGNWHC DSQWLGDRVI | 240 |
| | DLC- | TTSTRTWALP TYNNHLYKQI SNSTSGGSSN DNAYFGYSTP WGYFDENRFH CHFSPRDWQR | 300 |
| | AS1 | LINNNWGFRP KRLNFKLENI QVKEVTDNNG VKTIANNLTS TVQVFTDSDY QLPYVLGSAH | 360 |
| | (588_589) | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQSI TLVKSTQTVA | 600 |
| | | QAQTGWVQNQ GILPGMVWQD RDVYLQGPIW AKIPHTDGNF HPSPLMGGFG MKHPPPQILI | 660 |
| | | KNTPVPADPP TAFNKDKLNS FITQYSTGQV SVEIEWELQK ENSKRWNPEI QYTSNYYKSN | 720 |
| | | NVEFAVNTEG VYSEPRPIGT RYLTRNL (SEQ ID NO: 44) | |
| AAV9.588 | TILSRS | MAADGYLPDW LEDNLSEGIR EWWALKPGAP QPKANQQHQD NARGLVLPGY KYLGPGNGLD | 60 |
| Herp | TQTG | KGEPVNAADA AALEHDKAYD QQLKAGDNPY LKYNHADAEF QERLKEDTSF GGNLGRAVFQ | 120 |
| (9 588 | (SEQ ID | AKKRLLEPLG LVEEAAKTAP GKKRPVEQSP QEPDSSAGIG KSGAQPAKKR LNFGQTGDTE | 180 |
| Hep) | NO: 15), | SVPDPQPIGE PPAAPSGVGS LTMASGGGAP VADNNEGADG VGSSSGNWHC DSQWLGDRVI | 240 |
| | DLC- | TTSTRTWALP TYNNHLYKQI SNSTSGGSSN DNAYFGYSTP WGYFDENRFH CHFSPRDWQR | 300 |
| | AS2, | LINNNWGFRP KRLNFKLENI QVKEVTDNNG VKTIANNLTS TVQVFTDSDY QLPYVLGSAH | 360 |
| | 588_589 | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQTI LSRSTQTGAQ | 600 |
| | | AQTGWVQNQG ILPGMVWQDR DVYLQGPIWA KIPHTDGNFH PSPLMGGFGM KHPPPQILIK | 660 |
| | | NTPVPADPPT AFNKDKLNSF ITQYSTGQVS VEIEWELQKE NSKRWNPEIQ YTSNYYKSNN | 720 |
| | | VEFAVNTEG VYSEPRPIGT RYLTRNL (SEQ ID NO: 45) | |

| | | | |
|---|---|---|---|
| AAVP | SITLVK | 1 | MAADGYLPDW LEDNLSEGIR EWWALKPGAP QPKANQQHQD NARGLVLPGY KYLGPGNGLD |
| HPeB. | STQTV | 61 | KGEPVNAADA AALEHDKAYD QQLKAGDNPY LKYNHADAEF QERLKEDTSF GGNLGRAVFQ |
| VP2Ad | (SEQ ID | 121 | AKKRLLEPLG LVEEAAKTTI LSRSTQTGAP GKKRPVEQSP QEPDSSAGIG KSGAQPAKKR |
| | NO: 14), | | LNFGQTGDTE |
| | DLC- | 191 | SVPDPQPIGE PPAAPSGVGS LTMASGGGAP VADNNEGADG VGSSSGNWHC DSQWLGDRVI |
| | AS1 | 251 | TTSTRTWALP TYNNHLYKQI SNSTSGGSSN DNAYFGYSTP WGYFDENRFH CHFSPRDWQR |
| | (138_139) | 311 | LINNNWGFRP KRLNFKLFNI QVKEVTDNNG VKTIANNLTS TVQVFTDSDY QLPYVLGSAH |
| | | 371 | EGCLPPFPAD VFMIPQYGYL TLNDGSQAVG RSSFYCLEYF PSQMLRTGNN FQFSYEFENV |
| | | 431 | PFHSSYAHSQ SLDRLMNPLI DQYLYYLSRT INGSGQNQQT LKFSVAGPSN MAVQGRNYIP |
| | | 491 | GPSYRQQRVS TTVTQNNNSE FAWPGASSWA LNGRNSLMNP GPAMASHKEG EDRFFPLSGS |
| | | 551 | LIFGKQGTGR DNVDADKVMI TNEEEIKTTN PVATESYGQV ATNHQSDGTL AVPFKAQAQT |
| | | 611 | GWVQNQGILP GMVWQDRDVY LQGPIWAKIP HTDGNFHPSP LMGGFGMKHP PPQILIKNTP |
| | | 671 | VPADPPTAFN KDKLNSFITQ YSTGQVSVEI EWELQKENSK RWNPEIQYTS NYYKSNNVEF |
| | | 731 | AVNTEGVYSE PRPIGTRYLT RNL (SEQ ID NO: 46) |
| AAVP | TILSRS | 1 | MAADGYLPDW LEDNLSEGIR EWWALKPGAP QPKANQQHQD NARGLVLPGY KYLGPGNGLD |
| HPeB. | TQTG | 61 | KGEPVNAADA AALEHDKAYD QQLKAGDNPY LKYNHADAEF QERLKEDTSF GGNLGRAVFQ |
| VP2Herp | (SEQ ID | 121 | AKKRLLEPLG LVEEAAKTSI TLVKSTQTVA PGKKRPVEQS PQEPDSSAGI GKSGAQPAKK |
| | NO: 15), | | RLNFGQTGDT E |
| | DLC- | 192 | SVPDPQPIGE PPAAPSGVGS LTMASGGGAP VADNNEGADG VGSSSGNWHC DSQWLGDRVI |
| | AS2, | 252 | TTSTRTWALP TYNNHLYKQI SNSTSGGSSN DNAYFGYSTP WGYFDENRFH CHFSPRDWQR |
| | (138_139 | 312 | LINNNWGFRP KRLNFKLFNI QVKEVTDNNG VKTIANNLTS TVQVFTDSDY QLPYVLGSAH |
| | | 372 | EGCLPPFPAD VFMIPQYGYL TLNDGSQAVG RSSFYCLEYF PSQMLRTGNN FQFSYEFENV |
| | | 432 | PFHSSYAHSQ SLDRLMNPLI DQYLYYLSRT INGSGQNQQT LKFSVAGPSN MAVQGRNYIP |
| | | 492 | GPSYRQQRVS TTVTQNNNSE FAWPGASSWA LNGRNSLMNP GPAMASHKEG EDRFFPLSGS |
| | | 552 | LIFGKQGTGR DNVDADKVMI TNEEEIKTTN PVATESYGQV ATNHQSDGTL AVPEKAQAQT |
| | | 612 | GWVQNQGILP GMVWQDRDVY LQGPIWAKIP HTDGNFHPSP LMGGFGMKHP PPQILIKNTP |
| | | 672 | VPADPPTAFN KDKLNSFITQ YSTGQVSVEI EWELQKENSK RWNPEIQYTS NYYKSNNVEF |
| | | 732 | AVNTEGVYSE PRPIGTRYLT RNL (SEQ ID NO: 47) |

| Rh.64R1 | R697W | MAADGYLPDW LEDNLSEGIR EWWDLKPGAP KPKANQQKQD DGRGLVLPGY | |
|---|---|---|---|
| | | KYLGPFNGLD KGEPVNAADA AALEHDKAYD QQLKAGDNPY LRYNHADAEF | |
| | | QERLQEDTSF GGNLGRAVFQ AKKRVLEPLG LVEEGAKTAP GKKRPVEPSP | |
| | | QRSPDSSTGI GKKGQQPARK RLNFGQTGDS ESVPDPQPIG EPPAAPSSVG | |
| | | SGTMAAGGGA PMADNNEGAD GVGSSSGNWH CDSTWLGDRV ITTSTRTWAL | |
| | | PTYNNHLYKQ ISNGTSGGST NDNTYFGYST PWGYFDENRF HCHFSPRDWQ | |
| | | RLINNNWGFR PKRLSFKLEN IQVKEVTQNE GTKTIANNLT STIQVFTDSE | |
| | | YQLPYVLGSA HQGCLPPFPA DVFMIPQYGY LTLNNGSQAV GRSSFYCLEY | |
| | | FPSQMLRTGN NFSFSYTFED VPFHSSYAHS QSLDRLMNPL IDQYLYYLSR | |
| | | TQSTGGTAGT QQLLFSQAGP SNMSAQARNW LPGPCYRQQR VSTTLSQNNN | |
| | | SNFAWTGATK YHLNGRDSLV NPGVAMATNK DDEDRFFPSS GILMFGKQGA | |
| | | GKDNVDYSNV MLTSEEEIKT TNPVATEQYG VVADNLQQQN TAPIVGAVNS | |
| | | QGALPGMVWQ NRDVYLQGPI WAKIPHTDGN FHPSPLMGGF GLKHPPPQIL | |
| | | IKNTPVPADP PTAFNQAKLN SFITQYSTGQ VSVEIVWELQ KENSKRWNPE (SEQ ID NO: 48) | |
| AAV9. | N272A | MAADGYLPDW LEDNLSEGIR EWWALKPGAP QPKANQQHQD NARGLVLPGY KYLGPGNGLD | 60 |
| N272A.496N | 498_NNN/ | KGEPVNAADA AALEHDKAYD QQLKAGDNPY LKYNHADAEF QERLKEDTSF GGNLGRAVFQ | 120 |
| NN/AAA498 | AAA_500 | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DAAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAWPGASSWA LNGRNSLMNP GPAMASHKEG EDRFFPLSGS | 540 |
| | | LIFGKQGTGR DNVDADKVMI TNEEEIKTTN PSPLMGGFGM KHPPPQILIK NTPVPADPPT | 600 |
| | | ILPGMVWQDR DVYLQGPIWA KIPHTDGNFH NSKRWNPEIQ YTSNYYKSNN VEFAVNTEGV | 660 |
| | | AFNKDKLNSF ITQYSTGQVS VEIEWELQKE PVATESYGQV ATNHQSAQAQ AQTGWVQNQG | 720 |
| | | YSEPRPIGTR YLTRNL (SEQ ID NO: 49) | |
| AAV9. | G266A | MAADGYLPDW LEDNLSEGIR EWWALKPGAP QPKANQQHQD NARGLVLPGY KYLGPGNGLD | 60 |
| G266A.496N | 498_NNN/ | KGEPVNAADA AALEHDKAYD QQLKAGDNPY LKYNHADAEF QERLKEDTSF GGNLGRAVEQ | 120 |
| NN/AAA498 | AAA_500 | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSAGSSN DNAYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 50) | |
| AAV9. | | MAADGYLPDW LEDNLSEGIR EWWALKPGAP QPKANQQHQD NARGLVLPGY KYLGPGNGLD | 60 |
| 496NNN/ | | KGEPVNAADA AALEHDKAYD QQLKAGDNPY LKYNHADAEF QERLKEDTSF GGNLGRAVFQ | 120 |
| AAA498. | | AKKRLLEPLG LVEEAAKTAP GKKRPVEQSP QEPDSSAGIG KSGAQPAKKR LNFGQTGDTE | 180 |
| W503A | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLENI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAAPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 51) | |

| | | |
|---|---|---|
| rh34 | — | MAADGYLPDWLEDNLSEGIREWWDLKPGAPKPKANQQKQDDGRGLVLPGYEYLGPENGLDKGEPVNAADA |
| | | AALEHDKAYDQQLKAGDNPYLRYNHADAEFQERLQEDTSFGGNLGRAVFQAKKRVLEPLGLVEEGAKTAP |
| | | GKKRPLESPQEPDSSSGIGKKGKQPAKKRLNFEEDTGAGDGPPEGSDTSAMSSDIEMRAAPGGNAVDAGQ |
| | | GSDGVGNASGDWHCDSTWSEGKVTTTSTRTWVLPTYNNHLYLRLGTTSNSNTYNGFSTPWGYFDFN |
| | | RFHCHFSPRDWQRLINNNWGLRPKAMRVKIFNIQVKEVTTSNGETTVANNLTSTVQIFADSSYELPYVMD |
| | | AGQEGSLPPFPNDVFMVPQYGYCGIVTGENQNQTDRNAFYCLEYFPSQMLRTGNNFETAYNFEKVPFHSM |
| | | YAHSQSLDGLMNPLLDQYLWHLQSTTSGETLNQGNAATTFGKIRSGDFAFYRKNWLPGPCVKQQRFSKTA |
| | | SQNYKIPASGGNALLKYDTHYTLNNRWSNIAPGPPMATAGPSDGDFSNAQLIFPGPSVTGNTTTSANNLL |
| | | FTSEEEIAATNPRDTDMFGQIADNNQNATTAPITGNVTAMGVLPGMVWQNRDIYYQGPIWAKIPHADGHE |
| | | HPSPLIGGFGLKHPPPQIFIKNTPVPAYPATTFTAARVDSFITQYSTGQVAVQIEWEIEKERSKRWNPEV |
| | | QFTSNCGNQSSMLWAPDTTGKYTEPRVIGSRYLTNHL (SEQ ID NO: 88) |
| hu.31 | — | MAADGYLPDW LEDTLSEGIR QWWKLKPGPP PPKPAERHKD DSRGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGSQPAKKK LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDENRFH CHFSPRDWQR |
| | | LINNNWGFRP KRLNFKLENI QVKEVTDNNG VKTIANNLTS TVQVFTDSDY |
| | | QLPYVLGSAH EGCLPPFPAD VFMIPQYGYL TLNDGGQAVG RSSFYCLEYF |
| | | PSQMLRTGNN FQFSYEFENV PFHSSYAHSQ SLDRLMNPLI DQYLYYLSKT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSAQAQ AQTGWVQNQG |
| | | ILPGMVWQDR DVYLQGPIWA KIPHTDGNFH PSPLMGGFGM KHPPPQILIK |
| | | NTPVPADPPT AFNKDKLNSF ITQYSTGQVS VEIEWELQKE NSKRWNPEIQ |
| | | YTSNYYKSNN VEFAVSTEGV YSEPRPIGTR YLTRNL (SEQ ID NO: 89) |
| rh.31 | — | KAYDQQLKAG DNPYLRYNHA DAEFQERLQE DTSFGGNLGR AVFQAKKRVL |
| | | EPLGLVETPA KTAPGKKRPV DSPDSTSGIG KKGQQPAKKR LNFGQTGDSE |
| | | SVPDPQPIGE PPAGPSGLGS GTMAAGGGAP MADNNEGADG VGNASGNWHC |
| | | DSTWLGDRVI TTSTRTWALP TYNNHLYKQI SSQSAGSTND NVYFGYSTPW |
| | | GYFDFNRFHC HFSPRDWQRL INNNWGFRPK KLNFKLENIQ VKEVTTNDGV |
| | | TTIANNLTST VQVFSDSEYQ LPYVLGSAHQ GCLPPFPADV FMIPQYGYLT |
| | | LNNGSQSVGR SSFYCLEYFP SQMLRTGNNF TFSYTFEDVP FHSSYAHSQS |
| | | LDRLMNPLID QYLYYLARTQ SNAGGTAGNR ELQFYQGGPT TMAEQAKNWL |
| | | PGPCFRQQRV SKILDQNNNS NFAWTGATKY HLNXRNSLVN PGVAMATHKD |
| | | DEERFFPSSG VLIFGKTGAA NKTTLENVLM TNEEEIRPTN PVATEEYGIV |
| | | SSNLQAASTA AQTQVVNNQG ALPGMVWQNR DVYLQGPIWA KIPHTDGNFH |
| | | PSPLMGGFGL KHPPPQILIK NTPVPANPPE VFTPAKFASF ITQYSTGQVS |
| | | VEIEWELQKE NSKRWNPEIQ YTSNEDKQTG VDFAVDSQGV YSEP (SEQ ID NO: 90) |
| hu.12 | — | MAADGYLPDWLEDTLSEGIRQWWKLKPGPPPPKPAERHQDDSRGLVLPGYKYLGPENGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGHQPARKRLNFGQTGDAD |
| | | SVPDPQPLGQPPAAPTSLGSTTMATGSGAPMADNNEGADGVGNSSGNWHCDSQWLGDRVI |
| | | TTSTRTWALPTYNNHLYKQISSQSGASNDNHYFGYSTPWGYFDFNRFHCHFSPRDWQRLI |
| | | NNNWGFRPKRLNFKLFNIQVKEVTQNDGTTTIANNLTSTVQVFTDSEYQLPYVLGSAHQG |
| | | CLPPFPADVFMVPQYGYLTLNNGSQAVGRPSFYCLEYFPSQMLRTGNNFTFSYTFEDVPF |
| | | HSSYAHSQSLDRLMNPLIDQYLYYLNRTQSNSGTLQQSRLLFSQAGPTSMSLQAKNWLPG |
| | | PCYRQQRLSKQANDNNNSNFPWTAATKYHLNGRDSLVNPGPAMASHKDDEEKFFPMHGTL |
| | | IFGKQGTNANDADLEHVMITDEEEIRTTNPVATEQYGNVSNNLQNSNTGPTTENVNHQGA |
| | | LPGMVWQDRDVYLQGPIWAKIPHTDGHFHPSPLMGGFGLKHPPPQIMIKNTPVPANPPTN |
| | | FSSAKFASFITQYSTGQVSVEIEWELQKENSKRWNPEIQYTSNYNKSVNVDFTVDTNGVY |
| | | SEPRPIGTRYLTRNL (SEQ ID NO: 91) |
| hu.13 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF |
| | | QERLKEDTSF GGNLGRAVFQ AKKRVLEPLG LVEEPVKTAP GKKRPVEHSP |
| | | AEPDSSSGTG KAGQQPARKR LNFGQTGDAD SVPDPQPLGQ PPAAPSGLGT |
| | | NTMASGSGAP MADNNEGADG VGNSSGNWHC DSTWMGDRVI TTSTRTWALP |
| | | TYNNHLYKQI SSQSGASNDN HYFGYSTPWG YFDENRFHCH FSPRDWQRLI |
| | | NNNWGFRPKR LNFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS |
| | | QMLRTGNNFT FSYTFEDVPF HSSYAHSQSL DRLMNPLIDQ YLYYLSRTNT |
| | | PSGTTTQSRL QFSQAGASDI RDQSRNWLPG PCYRQQRVSK TSADNNNSEY |
| | | SWTGATKYHL NGRDSLVNPG PAMASHKDDE EKFFPQSGVL IFGKQGSEKT |
| | | NVDIEKVMIT DEEEIRTTNP VATEQYGSVS TNLQGGNTQA ATADVNTQGV |
| | | LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY |
| | | TSNYNKSVNV DFTVDINGVY SEPRPIGTRY LTRNL (SEQ ID NO: 92) |
| hu.21 | — | MAADGYLPDWLEDTLSEGIRQWWKLKPGPPPPKPAERHKDDSRGLVLPGYKYLGPENGLDKGEPVNEADA |
| | | AALEHDKAYDRQLDSGDNPYLKYNHADAEFQERLKEDTSFGGNLGRAVFQAKKRILEPLGLVEEPVKTAP |
| | | GKKRPVEHSPAEPDSSSGTGKAGQQPARKRLNFGQTGDADSVPDPRPLGQPPAAPSGLGTNTMASGSGAP |
| | | MADNNEGADGVGNSSGNWHCDSTWMGDRVITTSTRTWALPTYNNHLYKQISSQSGASNDNHYFGYSTPWG |
| | | YFDFNRFHCHFSPRDWQRLINNNWGFRPKRLSFKLFNIQVKEVTQNDGTTTIANNLTSTVQVFTDSEYQL |
| | | PYVLGSAHQGCLPPFPADVFMVPQYGYLTLNNGSQAVGRSSFYCLEYFPSQMLRTGNNFTFSYTFEDVPF |
| | | HSSYAHSQSLDRLMNPLIDQYLYYLSRTNTPSGTTTMSRLQFSQAGASDIRDQSRNWLPGPCYRQQRVSK |
| | | TAADNNNSDYSWTGATKYHLNGRDSLVNPGPAMASHKDDEEKYFPQSGVLIFGKQDSGKTNVDIEKVMIT |
| | | DEEEIRTTNPVATEQYGSVSTNLQSGNTQAATSDVNTQGVLPGMVWQDRDVYLQGPIWAKIPHTDGHFHP |
| | | SPLMGGFGLKHPPPQILIKNTPVPANPSTTFSAAKFASFITQYSTGQVSVEIEWELQKENSKRWNPEIQY |
| | | TSNYNKSVNVDFTVDTNGVYSEPRPIGTRYLTRNL (SEQ ID NO: 93) |
| hu.26 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF QERLKEDTSF |
| | | GGNLGRAVFQ AKKRILEPLG LVEEPVKTAP GKKRPVEHSP AEPDSSSGTG KAGQQPARKR |
| | | LNFGQTGDAD SVPDPQPLGQ PPAAPSGLGT NTMASGSGAP MADNNEGADG VGNSSGNWHC |
| | | DSTWMGDRVI TTSTRTWALP TYNNHLYKQI SSQSGASNDN HYFGYSTPWG YFDENRFHCH |
| | | FSPRDWQRLI NNNWGFRPKR LSFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS QMLRTGNNET |
| | | FSYTFEDVPF HSSYAHSQSL DRLMNPLIDQ YLYYLSRTNT PSGTTTMSRL QFSQAGASDI |
| | | RDQSRNWLPG PCYRQQRVSK TAADNNNSDY SWTGATKYHL NGRDSLVNPG PAMASHKDDE |
| | | EKYFPQSGVL IFGKQDSGKT NVDIEKVMIT DEEEIRTTNP VATEQYGSVS TNLQSGNTQA |
| | | ATSDVNTQGV LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY TSNYNKSVNV |
| | | DFTVDINGVY SEPRPIGTRY LTRNL (SEQ ID NO: 94) |
| hu.53 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF |
| | | QERLKEDTSF GGNLGRAVFQ AKKRVLEPLG LVEEPVKTAP GKKRPVEHSP |
| | | AEPDSSSGTG KAGQQPARKR LNFGQTGDAD SVPDPQPLRQ PPAAPTSLGS |
| | | TTMATGSGAP MADNNEGADG VGNSSGNWHC DSQWLGDRVI TTSTRTWALP |
| | | TYNNHLYKQI SSQSGASNDN HYFGYSTPWG YFDENRFHCH FSPRDWQRLI |
| | | NNNWGFRPKR LNFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS |
| | | QMLRTGNNFQ FSYTFEDVPF HSSYAHSQSL DRLMNPLIDQ YLYYLNRTQT |
| | | ASGTQQSRLL FSQAGPTSMS LQAKNWLPGP CYRQQRLSKQ ANDNNNSNFP |
| | | WTGATKYYLN GRDSLVNPGP AMASHKDDEE KFFPMHGTLI FGKEGTNATN |
| | | AELENVMITD EEEIRTTNPV ATEQYGYVSN NLQNSNTAAS TETVNHQGAL |
| | | PGMVWQDRDV YLQGPIWAKI PHTDGHFHPS PLMGGFGLKH PPPQIMIKNT |
| | | PVPANPPTNF SSAKFASFIT QYSTGQVSVE IEWELQKENS KRWNPEIQYT |
| | | SNYNKSVNVD FTVDINGVYS EPRPIGTRYL TRNL (SEQ ID NO: 95) |
| hu.56 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF |
| | | QERLKEDTSF GGNLGRAVFQ AKKRVLEPLG LVEEPVKTAP GKKRPVEHSP |
| | | VEPDSSSGTG KAGNQPARKR LNFGQTGDAD SVPDPQPLGQ PPASPSGLGT |
| | | NTMATGSGAP MADNNEGADG VGNSSGNWHC DSTWMGDRVV TTSTRTWALP |
| | | TYNNHLYKQI SSQSGASNDN HYFGYSTPWG YFDENRFHCH FSPRDWQRLI |
| | | NNNWGFRPKR LNFKLFNIQV KEVTQNDGTT TIANNLTSTV QVFTDLEYQL |
| | | PYVLGSAHQG CLPPFPADVF MVPQYGYLTL NNGSQAVGRS SFYCLEYFPS |
| | | QMLRTGNNFT FSYTFEDVPF HSSYAHSQSL DRLMNPLIDQ YLYYLSRINT |
| | | PSGTTTQSRL QFSQAGASDI RDQSRNWLPG PCYRQQRVSK TAADNNNSEY |
| | | SWTGATKYHL NGRDSLVNPG PAMASHKDDE EKFFPQSGVL IFGKQGSEKT |
| | | NVDIEKVMIT DEEEIRTTNP VATEQYGSVS TNLQSGNTQA ATSDVNTQGV |
| | | LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY |
| | | TSNYNKSVNV DFTVDINGVY SEPRPIGTRY LTRNL (SEQ ID NO: 96) |
| AAVrh.46 | | MAADGYLPDW LEDNLSEGIR EWWDLKPGAP KPKANQQKQD DGRGLVLPGY KYLGPENGLD |
| | | KGEPVNAADA AALEHDKAYD QQLKAGDNPY LRYNHADAEF QERLQEDTSF GGNLGRAVEQ |
| | | AKKRVLEPLG LVEEGAKTAP GKKRPVEPSP QRSPDSSTGI GKKGQQPARK RLNFGQTGDS |
| | | ESVPDPQPIG EPPAAPSSVG SGTMAAGGGA PMADNNEGAD GVGSSSGNWH CDSTWLGDRV |
| | | ITTSTRTWAL PTYNNHLYKQ ISNGTSGGST NDNTYFGYST PWGYFDENRF HCHFSPRDWQ |
| | | RLINNNWGFR PKRLSFKLEN IQVKEVTQNE GTKTIANNLT STIQVFTDSE YQLPYVLGSA |
| | | HQGCLPPFPA DVFMIPQYGY LTLNNGSQAV GRSSFYCLEY FPSQMLRTGN NFSFSYTFED |
| | | VPFHSSYAHS QSLDRLMNPL IDQYLYYLSR TQSTGGTAGT QQLLFSQAGP SNMSAQARNW |
| | | LPGPCYRQQR VSTTLSQNNN SNFAWTGATK YHLNGRDSLV NPGVAMATNK DDEDRFFPSS |
| | | GILMFGKQGA GKDNVDYSNV MLTSEEEIKA TNPVATEQYG VVADNLQQQN TAPIVGAVNS |
| | | QGALPGMVWQ NRDVYLQGPI WAKIPHTDGN FHPSPLMGGF GLKHPPPQIL IKNTPVPADP |
| | | PTAFNQAKLN SFITQYSTGQ VSVEIEWELQ KENSKRWNPE IQYTSNYYKS TNVDFAVNTE |
| | | GVYSEPRPIG TRYLTRNL (SEQ ID NO: 97) |

TABLE 5
Promoter Sequences

| Promoter Name | SEQ ID NO | Nucleic Acid Sequence |
|---|---|---|
| CAG | SEQ ID NO: 101 | GACATTGATTATTGACTAGTTATTAATAGTAATCAATTACGGGGTCATTAGTTCATA |
| | | GCCCATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTAACGC |
| | | CAATAGGGACTTTCCATTGACGTCAATGGGTGGAGTATTTACGGTAAACTGCCCACT |
| | | TGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTT |
| | | GGCAGTACATCTACGTATTAGTCATCGCTATTACCATGGTCGAGGTGAGCCCCACGT |
| | | TCTGCTTCACTCTCCCCATCTCCCCCCCCTCCCCACCCCCAATTTTGTATTTATTTA |
| | | TTTTTTAATTATTTTGTGCAGCGATGGGGGCGGGGGGGGGGGGGGGGCGCGCGCCAG |
| | | GCGGGGCGGGGCGGGGCGAGGGGCGGGGCGGGGCGAGGCGGAGAGGTGCGGCGGCAG |
| | | CCAATCAGAGCGGCGCGCTCCGAAAGTTTCCTTTTATGGCGAGGCGGCGGCGGCGGC |
| | | GGCCCTATAAAAAGCGAAGCGCGCGGCGGGCGGGAGTCGCTGCGCGCTGCCTTCGCC |
| | | CCGTGCCCCGCTCCGCCGCCGCCTCGCGCCGCCCGCCCCGGCTCTGACTGACCGCGT |
| | | TACTCCCACAGGTGAGCGGGCGGGACGGCCCTTCTCCTCCGGGCTGTAATTAGCGCT |
| | | TGGTTTAATGACGGCTTGTTTCTTTTCTGTGGCTGCGTGAAAGCCTTGAGGGGCTCC |
| | | GGGAGGGCCCTTTGTGCGGGGGGAGCGGCTCGGGGGGTGCGTGCGTGTGTGTGTGCG |
| | | TGGGGAGCGCCGCGTGCGGCTCCGCGCTGCCCGGCGGCTGTGAGCGCTGCGGGCGCG |
| | | GCGCGGGGCTTTGTGCGCTCCGCAGTGTGCGCGAGGGGAGCGCGGCCGGGGGCGGTG |
| | | CCCCGCGGTGCGGGGGGGGCTGCGAGGGGAACAAAGGCTGCGTGCGGGGTGTGTGCG |
| | | TGGGGGGGTGAGCAGGGGGTGTGGGCGCGTCGGTCGGGCTGCAACCCCCCCTGCACC |
| | | CCCCTCCCCGAGTTGCTGAGCACGGCCCGGCTTCGGGTGCGGGGCTCCGTACGGGGC |
| | | GTGGCGCGGGGCTCGCCGTGCCGGGCGGGGGGTGGCGGCAGGTGGGGGTGCCGGGCG |
| | | GGGCGGGGCCGCCTCGGGCCGGGGAGGGCTCGGGGGAGGGGCGCGGCGGCCCCCGGA |
| | | GCGCCGGCGGCTGTCGAGGCGCGGCGAGCCGCAGCCATTGCCTTTTATGGTAATCGT |
| | | GCGAGAGGGCGCAGGGACTTCCTTTGTCCCAAATCTGTGCGGAGCCGAAATCTGGGA |
| | | GGCGCCGCCGCACCCCCTCTAGCGGGCGCGGGGCGAAGCGGTGCGGCGCCGGCAGGA |
| | | AGGAAATGGGGGGGGAGGGCCTTCGTGCGTCGCCGCGCCGCCGTCCCCTTCTCCCTC |
| | | TCCAGCCTCGGGGCTGTCCGCGGGGGGACGGCTGCCTTCGGGGGGGACGGGGCAGGG |
| | | CGGGGTTCGGCTTCTGGCGTGTGACCGGCGGCTCTAGAGCCTCTGCTAACCATGTTC |
| | | ATGCCTTCTTCTTTTTCCTACAGCTCCTGGGCAACGTGCTGGTTATTGTGCTGTCTC |
| | | ATCATTTTGGCAAAG |

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1.-39. (canceled)

40. A method for treating a subject diagnosed with a neurological disorder, said method comprising administering a therapeutically effective amount of a composition comprising a recombinant adeno-associated virus (rAAV) to the striatum of the subject, wherein the rAAV comprises a modified AAV9 capsid packaging a transgene suitable for treating the neurological disorder, and wherein the rAAV exhibits tropism for the putamen and/or caudate.

41. The method of claim 40 wherein the rAAV exhibits tropism for dopaminergic neurons.

42. The method of claim 40, wherein the rAAV exhibits retrograde or anterograde transport to the substantia nigra.

43. The method of claim 40, wherein the rAAV exhibits 5 to 10 fold improved transduction of putamen, caudate, substantia nigra, intralaminar thalamus and/or frontal cortex tissue relative to a rAAV with an AAV9 capsid.

44. The method of claim 40, wherein the transgene in transcribed at 4 to 6 fold higher levels in putamen and/or caudate tissue relative to the same transgene packaged in an AAV9 capsid.

45. A method for the delivery of a therapeutic product to dopaminergic neurons, comprising administering a composition comprising a recombinant adeno-associated virus (rAAV) into a brain region of a subject, wherein the brain region is innervated by dopaminergic neurons, and the rAAV comprises a modified AAV9 capsid packaging a transgene that expresses the therapeutic product in the dopaminergic neurons.

46. A method for the delivery of a therapeutic product to the substantia nigra, comprising administering a composition comprising a recombinant adeno-associated virus (rAAV) into a brain region of a subject in need thereof, wherein the brain region is innervated by dopaminergic neurons, and the rAAV comprises a modified AAV9 capsid packaging a transgene that expresses the therapeutic product in the substantia nigra.

47. The method of claim 40, wherein the composition is administered intraparenchymally.

48. (canceled)

49. The method of claim 40, wherein the modified AAV9 capsid comprises a modified capsid protein comprising a peptide insertion in variable region IV.

50. The method of claim 49, wherein the peptide is a TFR3 peptide RTIGPSV (SEQ ID NO:19).

51. The method of claim 49, wherein the peptide is inserted immediately after position S454 of the modified AAV9 capsid protein (SEQ ID NO:74).

52. The method of claim 51, wherein the modified AAV9 capsid protein is AAV9.S454-TFR3 (SEQ ID NO:42).

53. The method of claim 40, wherein the neurological disorder is Parkinson's Disease and/or levodopa-induced dyskinesia (LID) in Parkinson's Disease (PD-LID) and/or tardive dyskinesia.

54. The method of claim 45, wherein the composition is administered intraparenchymally.

55. The method of claim 45, wherein the modified AAV9 capsid protein comprises a peptide insertion in variable region IV.

56. The method of claim 55, wherein the peptide is a TFR3 peptide RTIGPSV (SEQ ID NO:19).

57. The method of claim 55, wherein the peptide is inserted after position S454 of the modified AAV9 capsid (SEQ ID NO:74).

58. The method of claim 57, wherein the modified AAV9 capsid protein is AAV9.S454-TFR3 (SEQ ID NO:42).

59. The method of claim 46, wherein the composition is administered intraparenchymally.

60. The method of claim 46, wherein the modified AAV9 capsid protein comprises a peptide insertion in variable region IV.

61. The method of claim 60, wherein the peptide is a TFR3 peptide RTIGPSV (SEQ ID NO:19).

62. The method of claim 61, wherein the peptide is inserted after position S454 of the modified AAV9 capsid protein (SEQ ID NO:74).

63. The method of claim 62, wherein the modified AAV9 capsid protein is AAV9.S454-TFR3 (SEQ ID NO:42).


SEQUENCE LISTING (100 sequences)

SEQ ID NO: 1 - protein, 7 aa, synthetic construct
TLAAPFK

SEQ ID NO: 2 - protein, 8 aa, synthetic construct
DDDDDDDD

SEQ ID NO: 3 - protein, 7 aa, synthetic construct
LSSRLDA

SEQ ID NO: 4 - protein, 9 aa, synthetic construct
CLSSRLDAC

SEQ ID NO: 5 - protein, 7 aa, synthetic construct
CLPVASC

SEQ ID NO: 6 - protein, 5 aa, synthetic construct
LPVAS

SEQ ID NO: 7 - protein, 7 aa, synthetic construct
ASSLNIA

SEQ ID NO: 8 - protein, 6 aa, synthetic construct
QAQAQT

SEQ ID NO: 9 - protein, 4 aa, synthetic construct
STQT

SEQ ID NO: 10 - protein, 7 aa, synthetic construct
HAIYPRH

SEQ ID NO: 11 - protein, 12 aa, synthetic construct
THRPPMWSPVWP

SEQ ID NO: 12 - protein, 7 aa, synthetic construct
RTIGPSV

SEQ ID NO: 13 - protein, 9 aa, synthetic construct
CRTIGPSVC

SEQ ID NO: 14 - protein, 11 aa, synthetic construct
SITLVKSTQTV

SEQ ID NO: 15 - protein, 10 aa, synthetic construct
TILSRSTQTG

SEQ ID NO: 16 - protein, 7 aa, synthetic construct
QAVRTSL

SEQ ID NO: 17 - protein, 7 aa, synthetic construct
QAVRTSH

SEQ ID NO: 18 - protein, 20 aa, synthetic construct
VVMVGEKPITITQHSVETEG

SEQ ID NO: 19 - protein, 12 aa, synthetic construct
RSSEEDKSTQTT

SEQ ID NO: 20 - protein, 7 aa, synthetic construct
TLAVPFK

SEQ ID NO: 21 - protein, 8 aa, synthetic construct
DYKDDDDK

SEQ ID NO: 22 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 27 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQAAASNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQAAASNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 31 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVAGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 744 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDDDDDDDDGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLSSRLDAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 745 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLSSRLDACGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 741 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLPVASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLPVASCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASSLNIAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHAIYPRHGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRTIGPSVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 745 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCRTIGPSVCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 747 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSITLVKSTQTVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 746 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTILSRSTQTGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 753 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTTILSRSTQTGAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 754 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTSITLVKSTQTVAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 700 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPE

SEQ ID NO: 49 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDAAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 50 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSAGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAAPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 35 aa, synthetic construct
YYLSRTQNTGGTAXXQTLLFSQXGPSNMSXQAKNW

SEQ ID NO: 53 - protein, 35 aa, synthetic construct
YYLNRTQNQSGSAQNKDLLFSRGSPAGMSVQPKNW

SEQ ID NO: 54 - protein, 36 aa, synthetic construct
YYLSRTNCTPSGTTTQSRLQFSQAGASDIRDQSRNW

SEQ ID NO: 55 - protein, 36 aa, synthetic construct
YYLNRTQGTTSGTTNQSRLLFSQAGPQSMSLQARNW

SEQ ID NO: 56 - protein, 36 aa, synthetic construct
WGLQSTTTGTTLNAGTATTNFTKLRPTNFSNFKKNW

SEQ ID NO: 57 - protein, 29 aa, synthetic construct
YRFVSTNNTGGVQFNKNLAGRYANTYKNW

SEQ ID NO: 58 - protein, 35 aa, synthetic construct; identical to SEQ ID NO: 53

SEQ ID NO: 59 - protein, 36 aa, synthetic construct
YYLARTQSNPGGTAGNRELQFYQGGPSTMAEQAKNW

SEQ ID NO: 60 - protein, 35 aa, synthetic construct
YYLSRTQTTGGTANTQTLGFSQGGPNTMANQAKNW

SEQ ID NO: 61 - protein, 34 aa, synthetic construct
YYLSKTINGSGQNQQTLKFSVAGPSNMAVQGRNY

SEQ ID NO: 62 - protein, 35 aa, synthetic construct
YYLSRTQSTGGTAGTQQLLFSQAGPNNMSAQAKNW

SEQ ID NO: 63 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 64 - protein, 735 aa, synthetic construct [sequence omitted]

SEQ ID NO: 65 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 66 - protein, 736 aa, synthetic construct; identical to SEQ ID NO: 65

SEQ ID NO: 67 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAAPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 69 - protein, 734 aa, synthetic construct; identical to SEQ ID NO: 68

SEQ ID NO: 70 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 71 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 72 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 73 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 74 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 75 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 76 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 77 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 78 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 79 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSTLAAPFKSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 80 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 81 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 82 - protein, 735 aa, synthetic construct
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 83 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 84 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 85 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 86 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTLAAPFKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 87 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 88 - protein, 733 aa, synthetic construct
MAADGYLPDWLEDNLSEGIREWWDLKPGAPKPKANQQKQDDGRGLVLPGYEYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFETAYNFEKVPFHSMYAHSQSLDGLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPAYPATTFTAARVDSFITQYSTGQVAVQIEWEIEKERSKRWNPEVQFTSNCGNQSSMLWAPDTTGKYTEPRVIGSRYLTNHL

SEQ ID NO: 89 - protein, 736 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 90 - protein, 644 aa, synthetic construct
KAYDQQLKAGDNPYLRYNHADAEFQERLQEDTSFGGNLGRAVFQAKKRVLEPLGLVETPAKTAPGKKRPVDSPDSTSGIGKKGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSQSAGSTNDNVYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNAGGTAGNRELQFYQGGPTTMAEQAKNWLPGPCFRQQRVSKTLDQNNNSNFAWTGATKYHLNXRNSLVNPGVAMATHKDDEERFFPSSGVLIFGKTGAANKTTLENVLMTNEEEIRPTNPVATEEYGIVSSNLQAASTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKQTGVDFAVDSQGVYSEP

SEQ ID NO: 91 - protein, 735 aa, synthetic construct; identical to SEQ ID NO: 82

SEQ ID NO: 92 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 93 - protein, 735 aa, synthetic construct; identical to SEQ ID NO: 83

SEQ ID NO: 94 - protein, 735 aa, synthetic construct; identical to SEQ ID NO: 84

SEQ ID NO: 95 - protein, 734 aa, synthetic construct; identical to SEQ ID NO: 87

SEQ ID NO: 96 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPASPSGLGTNTMATGSGAPMADNNEGADGVGNSSGNWHCDSTWMGDRVVTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDLEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTAADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 97 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 98 - protein, 743 aa, synthetic construct; identical to SEQ ID NO: 67

SEQ ID NO: 99 - protein, 743 aa, synthetic construct; identical to SEQ ID NO: 79

SEQ ID NO: 100 - protein, 743 aa, synthetic construct; identical to SEQ ID NO: 86